(19) United States
(12) Patent Application Publication
(10) Pub. No.: US 2005/0282818 A1
(43) Pub. Date: Dec. 22, 2005

(54) Ubiquitin ligase inhibitors

(75) Inventors: Usha Ramesh, Cupertino, CA (US); Gary Look, Santa Clara, CA (US); Jianing Huang, Santa Clara, CA (US); Rajinder Singh, Belmont, CA (US); Richard Brent Mattis JR., Berkeley, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Rigel Pharmaceuticals, Inc., South San Francisco, CA (US)

(21) Appl. No.: 11/158,957

(22) Filed: Jun. 22, 2005

Related U.S. Application Data
(60) Provisional application No. 60/582,261, filed on Jun. 22, 2004.
(60) Provisional application No. 60/646,102, filed on Jan. 21, 2005.

(51) Int. Cl. A61K 31/496; A61K 31/4245; C07D 413/02

(52) U.S. Cl. 514/254.03; 514/364; 544/368

(57) ABSTRACT

This invention describes compounds and pharmaceutical compositions useful as ubiquitin agent inhibitors, particularly ubiquitin ligase inhibitors. The compounds and pharmaceutical compositions of the invention are useful as inhibitors of the biochemical pathways of organisms in which ubiquitination is involved, such as signal transduction pathways. The invention also comprises the use of the compounds and pharmaceutical compositions of the invention for the treatment of conditions that require inhibition of ubiquitination. Furthermore, the invention comprises methods of inhibiting ubiquitination in a cell comprising contacting a cell in which inhibition of ubiquitination is desired with a compound or pharmaceutical composition according to the invention. Particularly, the compounds and pharmaceutical compositions are useful to inhibit the ubiquitin ligase activity of TRAF6.

This application claims the benefit of U.S. Provisional Application No. 60/582,261, filed Jun. 22, 2004, and U.S. Provisional Application No. 60/646,102, filed Jan. 21, 2005.

### BACKGROUND OF THE INVENTION

1. Field of the Invention

This invention relates to the inhibition of ubiquitination. More particularly, the invention relates to compounds and methods for inhibiting ubiquitin ligase activity.

2. Summary of the Related Art

Ubiquitin is a 76 amino acid protein present throughout the eukaryotic kingdom. It is a highly conserved protein and is essentially the identical protein in diverse organisms ranging from humans to yeasts to fruit flies. In eukaryotes, ubiquitination is the key component of the ATP-dependent pathway for protein degradation and cellular regulatory processes. Proteins slated for degradation or that act as regulatory agents are covalently linked to ubiquitin via an ATP-dependent process catalyzed by three separate enzymes.

The ubiquitination of these target proteins is known to be mediated by the enzymatic activity of three ubiquitin agents. Ubiquitin is first activated in an ATP-dependent manner by a ubiquitin activating agent, for example, an E1. The C-terminus of a ubiquitin forms a high energy thioester bond with the ubiquitin activating agent. The ubiquitin is then transferred to a ubiquitin conjugating agent, for example, an E2 (also called ubiquitin moiety carrier protein), also linked to this second ubiquitin agent via a thioester bond. The ubiquitin is finally linked to its target protein (e.g. substrate) to form a terminal isopeptide bond under the guidance of a ubiquitin ligating agent, for example, an E3. In this process, monomers or oligomers of ubiquitin are attached to the target protein. On the target protein, each ubiquitin is covalently ligated to the next ubiquitin through the activity of a ubiquitin ligating agent to form polymers of ubiquitin.

Typically, the ubiquitination of target proteins by E3 in cells results in the formation of poly-ubiquitin chains. An isopeptide bond is formed between the carboxyl terminus of the ubiquitin and the ε-amino group of Lys in the target protein. The extension or formation of ubiquitin chains results from the formation of additional isopeptide bonds with the Lys⁴⁸ (and sometimes Lys⁶³) of a previously conjugated ubiquitin and the carboxyl-terminal Gly of an additional ubiquitin. The efficient recognition of a ubiquitinated target protein by a proteosome requires at least four ubiquitins linked in this configuration. However, in the case of Mdm2-mediated ubiquitination of p53, neither Lys⁴⁸ or Lys⁶³ is involved in the formation of poly-ubiquitin chains. Recent studies show that human Mdm2 mediates multiple mono-ubiquitination of p53 by a mechanism requiring enzyme isomerization (Zhihong et al. (2001) J. Biol. Chem. 276:31,357-31,367). Further, in vitro, the transfer of ubiquitin to p53 can occur independent of E1 when using an E2 pre-conjugated with ubiquitin. These results suggest that the pre-conjugated E2 can bind to Mdm2 and thereafter transfer the ubiquitin to the Mdm2 in the absence of an E1.

The enzymatic components of the ubiquitination pathway have received considerable attention (for a review, see Wong et al., DDT 8:746-754 (2003); Weissman, Nature Reviews 2:169-178 (2001)). The members of the E1 ubiquitin activating agents and E2 ubiquitin conjugating agents are structurally related and well characterized enzymes. There are numerous species of E2 ubiquitin conjugating agents, some of which act in preferred pairs with specific E3 ubiquitin ligating agents to confer specificity for different target proteins.

The family of ubiquitin and ubiquitin-like modifiers include ubiquitin, NEDD8, ISG15, SUM01, SUM02, SUM03, APG12, and APG8. Further, genome mining efforts have identified at least 530 human genes that encode enzymes responsible for ubiquitin conjugation and deconjugation. Many of these genes encode multiple splice variants, thereby increasing the diversity of enzyme families regulating ubiquitin conjugation and deconjugation. There are a multitude of E3's, reflecting their role as specificity determinants, an intermediate number of E2's, and few E1's, which are redundant to multiple pathways. Thus, the same E2 in conjunction with different E3's recognizes distinct substrates. For example, ubiquitin and ubiquitin-like enzymes are encodes by at least 11 genes that comprise at least 11 isoforms; E1's are encoded by at least 13 genes that include at least 15 isoforms; E2's, which include Ubc (ubiquitin carrier proteins) and Uev (ubiquitin enzyme variants), are encoded by at least 49 genes that comprise at least 77 isoforms; E3's, which include RING, PHD, HECT and U-box domain containing proteins, are encoded by at least 391 genes that comprise at least 631 isoforms; and DUB's (de-ubiquitylating enzymes), which includes USP, ULP, JAMM and UCH proteases, are encoded by at least 86 genes that comprise at least 136 isoforms. Ubiquitin conjugation and deconjugation pathways regulate diverse biological pathways. For example, ubiquitin conjugation and deconjugation pathways play important roles in cancers, inflammation, metabolism, viral diseases and central nervous system disorders. Thus, compounds that can modulate ubiquitin conjugation and deconjugation processes would serve as important therapeutic agents. For a more detailed review of ubiquitin regulatory pathways see Wong et al. (DDT 8 (16), 746-754 (2003)).

Generally, ubiquitin ligating agents contain two separate activities: a ubiquitin ligase activity to attach, via an isopeptide bond, monomers or oligomers of ubiquitin to a target protein, and a targeting activity to physically bring the ligase and substrate together. The substrate specificity of different ubiquitin ligating agents is a major determinant in the selectivity of the ubiquitin-mediated protein degradation process.

In eukaryotes, some ubiquitin ligating agents contain multiple subunits that form a complex having ubiquitin ligating activity. Particularly well characterized examples are SCFs, which play an important role in regulating Gi progression and consists of at least three subunits: SKP1, Cullins (having at least seven family members) and an F-box protein (of which hundreds of species are known) which bind directly to and recruit the substrate to the complex. The combinatorial interactions between the SCF's and a recently discovered family of RING finger proteins, the ROC/APC11 proteins, have been shown to be the key elements conferring ligase activity to ubiquitin ligating agents. Particular ROC/Cullin combinations can regulate specific cellular pathways, as exemplified by the function of APC11-APC2, involved in the proteolytic control of sister chromatid separation and exit from telophase into GI in mitosis (see King et al., supra; Koepp et al., Cell 97:431-34 (1999)), and ROC1-Cullin 1, involved in the proteolytic degradation of IKB in NF-KB/IKB mediated transcription regulation (Tan et al., Mol. Cell 3(4):527-533 (1999); Laney et al, Cell 97:427-30 (1999)).

The best characterized ubiquitin ligating agent is the APC (anaphase promoting complex), which is multi-component complex that is required for both entry into anaphase as well as exit from mitosis (see King et al., Science 274:1652-59 (1996) for review). The APC plays a crucial role in regulating the passage of cells through anaphase by promoting ubiquitin-mediated proteolysis of many proteins. In addition to degrading the mitotic B-type cyclin for inactivation of CDC2 kinase activity, the APC is also required for degradation of other proteins for sister chromatid separation and spindle disassembly. Most proteins known to be degraded by the APC contain a conserved nine amino acid motif known as the “destruction box” that targets them for ubiquitin ubiquitination and subsequent degradation. However, proteins that are degraded during GI, including GI cyclins, CDK inhibitors, transcription factors and signaling intermediates, do not contain this conserved amino acid motif. Instead, substrate phosphorylation appears to play an important role in targeting their interaction with a ubiquitin ligating agent for ubiquitination (see Hershko et al., Ann. Rev. Biochem. 67:429-75 (1998)).

Two major classes of E3 ubiquitin ligating agents are known: the HECT (homologous to E6-AP carboxy terminus) domain E3 ligating agents; and the RING finger domain E3 ligating agents. E6AP is the prototype for the HECT domain subclass of E3 ligating agents and is a multi-subunit complex that functions as a ubiquitin ligating agent for the tumor suppressor p53 which is activated by papillomavirus in cervical cancer (Huang et al. (1999) Science 286:1321-1326). Members of this class are homologous to the carboxyl terminus of E6AP and utilize a Cys active site to form a thiolester bond with ubiquitin, analogous to the E1 activating agents and E2 conjugating agents. However, in contrast, the members of the RING finger domain class of E3 ligating agents are thought to interact with an ubiquitin-conjugated-E2 intermediate to activate the complex for the transfer of ubiquitin to an acceptor. Examples of the RING domain class of E3 ligating agents are TRAF6, involved in IKK activation; Cbl, which targets insulin and EGF; Sina/Siah, which targets DCC; Itchy, which is involved in haematopoesis (B, T and mast cells); IAP, involved with inhibitors of apoptosis; and Mdm2 which is involved in the regulation of p53.

The RING finger domain subclass of E3 ligating agents can be further grouped into two subclasses. In one subclass, the RING finger domain and the substrate recognition domain are contained on different subunits of a complex forming the ubiquitin ligating agent (e.g., the RBx1 and the F-box subunit of the SCF complex). In the second subclass of ubiquitin ligating agents, the ligating agents have the RING finger domain and substrate recognition domain on a single subunit. (e.g., Mdm2 and cbl) (Tyers et al. (1999) Science 284:601, 603-604; Joazeiro et al. (2000) 102:549-552). A further class of ligating agents are those having a “PHD” domain and are homologs of the RING finger domain ligating agents (Coscoy et al. (2001) J. Cell Biol. 155(7):1265-1273), e.g., MEKK1. The PHD domain ligating agents are a novel class of membrane-bound E3 ligating agents.

In addition, a new class of ubiquitin ligases has been characterized. These are the U-box-containing proteins. (Patterson, Sci STKE 2002(116:PE4 (220)). This class, for the present, represents a small number of ligases which have yet to be extensively characterized.

Recently, an E2-E3 pair has been identified to consist of the UBC13-TRAF6 in conjuction with the Ubc-like protein Uev1A. This newly identified E2-E3 pair participates in many signal transduction pathways. TRAF6 (tumor necrosis factor (TNF) receptor associated factors) was first identified as intracellular proteins associated with TNF-R2 (reviewed by Wu et al., BioEssays 25, 1096-1105). Deng et al. (Cell 103, 351-361 (2000)) demonstrated that the RING domain of TRAF6, in conjunction with the ubiquitin-conjugating enzyme UBC 13 and the UBC-like protein Uev1A, exhibited ubiquitin ligase activity and catalyzed polyubiquitin chain formation linked by Lys-63, instead of Lys-48 of ubiquitin. It was later shown that the ubiquitin ligase activity of TRAF6 promotes poly-ubiquitin chains formation that activates TAK1 which in turn activates a number of important kinases such as IkB kinase and MAP kinases (reviewed by Wu et al., BioEssays 25, 1096-1105).

The ubiquitination of TRAF6 leads to activation of TAK1 which then activates IkB kinase. IkB kinase in turn activates the NF-kB pathway as well as phosphorylates MKK6 in the JNK-p38 kinase pathway. The NF-kB pathway includes many important processes such as inflammation, LPS-induces septic shock, viral infection such as HIV, and cell survival among others. Thus, the ubiquitin ligase activity of TRAF6 plays important regulatory roles in many cellular processes.

Ubiquitin agents, such as the ubiquitin activating agents, ubiquitin conjugating agents, and ubiquitin ligating agents, are key determinants not only in ubiquitin-mediated proteolytic pathway that results in the degradation of targeted proteins, but also in regulation of cellular processes. Consequently, agents that modulate the activity of such ubiquitin agents may be used to up-regulate or down-regulate specific molecules involved in cellular signal transduction. Disease processes can be treated by such up or down regulation of signal transducers to enhance or dampen specific cellular responses. This principle has been used in the design of a number of therapeutics, including phosphodiesterase inhibitors for airway disease and vascular insufficiency, kinase inhibitors for malignant transformation and proteasome inhibitors for inflammatory conditions such as arthritis.

Ubiquitin agents are involved in cancer, inflammation, adaptive immunity, innate immunity, bone metabolism, LPS-induced angiogenesis, osteoporosis, osteopinneal diseases, protozoan infection, viral infections, lymph node development, mammary gland development, skin development, and central nervous system development. Viral and protozoan infections involving ubiquitination include infections caused by variola viruses such as smallpox, HIV and related conditions, human papillomavirus, HSV, adenovirus, coxsackie virus, HCMV, KSHV, EBV, paramyxovirus, myxomavirus, ebola, retrovirus, rhabdovirus, and the malaria parasite. Other conditions in which ubiquitin is involved include aberrant cell growth, cancers, restenosis, psoriasis, and neoplastic cell proliferation. Ubiquitin is also involved in gene regulation, such as in bone metabolism, and in signal transduction pathways that involve IL-1, CD40, RANKL, LPS, IL-17, LMP1, NF-κB, AP-1 and kinases, such as MAP kinases, JNK/SAPK, ERK, p38, IkB kinase and Src-family tyrosine kinases. Further, ubiquitin plays a critical role in the TNFR/IL-1R/TLR signal transduction pathways of inflammation, for example, in autoimmune diseases such as rheumatoid arthritis (RA), chronic obstructive pulmonary disease (COPD), inflammatory bowel disease (IBD) and graft rejection, bone-destructive diseases, such as osteoporosis and RA, allergies and infective disease such as bacterial sepsis and associated systemic inflammation. Finally, ubiquitin plays a role in diseases and conditions that involve non-degradative ubiquitination, for example, in diseases and conditions that involve activation of K-63 linked, non-degradative ubiquitination.

Ubiquitin has also been implicated as key components in other biochemical processes. Ubiquitination of the Gag structural protein of Rous Sarcoma virus has been linked to the targeting of Gag to the cell membrane of the host cell where it can assemble into spherical particles and bud from the cell surface. Production of HIV particles has also been associated with ubiquitination and may constitute an important cellular pathway for producing infectious particles. Thus, the ubiquitin pathway may be an important target for treatment of HIV positive patients.

Due to the importance of ubiquitin-mediated proteolysis and regulation of cellular processes, for example cell cycle regulation and developmental processes and, consequently, disease states, there is a need for compounds that inhibit ubiquitin ligases. Such inhibitors can be used to inhibit and treat such diseases, and as research tools to identify the physiological role of ubiquitin ligases in various regulatory pathways and disease states.

### BRIEF SUMMARY OF THE INVENTION

The invention comprises compounds and compositions comprising the compounds for inhibiting ubiquitin agents. The compositions can further comprise a pharmaceutically acceptable carrier, diluent, and/or excipient and can be used in inhibiting and treating various conditions where ubiquitination is involved. They can also be used as research tools to study the role of ubiquitin in various natural and pathological processes.

In a first aspect, the invention comprises compounds that inhibit ubiquitination of target proteins.

In a second aspect, the invention comprises a pharmaceutical composition comprising an inhibitor of ubiquitination according to the invention and a pharmaceutically acceptable carrier, excipient, or diluent.

In a third aspect, the invention comprises methods of inhibiting ubiquitination in a cell, comprising contacting a cell in which inhibition of ubiquitination is desired with a pharmaceutical composition comprising a ubiquitin agent inhibitor according to the invention.

In a fourth aspect, the invention provides methods for treating cell proliferative diseases or conditions, comprising administering to a patient in need thereof a pharmaceutical composition comprising an effective amount of a ubiquitin agent inhibitor according to the invention.

In a fifth aspect, the invention provides methods for inhibiting TRAF6 activity in a cell, comprising administering to the cell a compound of the invention or a pharmaceutical composition comprising an effective amount of a compound according to the invention.

The foregoing only summarizes certain aspects of the invention and is not intended to be limiting in nature. These aspects and other aspects and embodiments are described more fully below. All patent applications and publications of any sort referred to in this specification are hereby incorporated by reference in their entirety. In the event of a discrepancy between the express disclosure of this specification and a patent application or publication incorporated by reference, the express disclosure of this specification shall control.

### DETAILED DESCRIPTION OF THE PREFERRED EMBODIMENTS

The invention provides compounds, compositions and methods for inhibiting ubiquitin ligase activity. In particular, the invention provides compounds, compositions and methods for inhibiting TRAF6, APC as well as other enzymes that exhibit E3-like activity. The invention also provides methods and compositions for treating cell proliferative diseases and conditions in which TRAF6 is involved.

In the first aspect, the invention provides compounds of Formula I 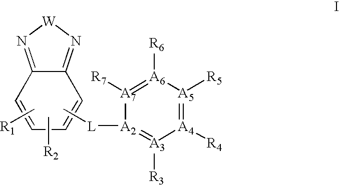
and pharmaceutically acceptable salts, hydrates, solvates, polymorphs, atrophisomers, N-oxides, and prodrugs thereof, wherein
(A):
L is covalent bond, —S—, —NH—, —SO₂—, —SO₃—, —S(O)—, —SO₂N(H)—, —SO₂N(H)—C₁-C₆ alkyl, —N(H)—C(O)—, —N(H)—C(O)—N(H)—, —N(H)SO₂—, —N(H)(C₁-C₆ alkyl)SO₂—;
W is —O— or —S(O)O-2; A₂, A₃, A₄, A₅, A₆ and A₇ are independently carbon or nitrogen provided that when any one of A₂, A₃, A₄, A₅, A₆ and A₇ is nitrogen the R moiety attached to it is absent, or when any one of A₂, A₃, A₄, A₅, A₆ and A₇ is nitrogen and the R moiety attached to it is present, then the nitrogen has a positive charge and is a quaternary ammonium;
R₁ and R₂ are independently —H, —NO₂, —OH, —CN, —SH, —S—C₁-C₆ alkyl-aryl, —S-aryl, —S-heteroaryl, —S—C-₁—C₆-alkyl-hetroaryl, —O—C₁-C₆ alkyl-aryl, —O—C₁-C₆ alkyl-heteroaryl, —O-aryl, —O-heteroaryl, —SO₂—OH, —S(O)—H, chloro, bromo, fluoro, iodo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, —N(R₈)Z, aryl, heterocyclyl, or heteroaryl, wherein each of the aryl, heterocyclyl, and heteroaryl is optionally substituted with 1 to 3 groups selected from —H, —OH, —SH, C₁-C₆ alkyl, C₁-C₆ alkoxy, mono- to per-halogenated C₁-C₆ alkyl, —NH₂, mono- or di-(C₁-C₆ alkyl) amino, —NO₂, halo, —CN, or aryl or heteroaryl each optionally substituted with mono- to per-halogenated C₁-C₆ alkyl, —OH, or CN;
R₈ is —H, C₁-C₆ alkyl, or C₁-C₆ alkoxy;
R₉ is —H or C₁-C₆ alkyl;
Z is aryl, heteroaryl, —C(O)—C₀-C₆ alkyl, —C(O)-heteroaryl, or —C(O)-aryl, —S(O)₂-aryl, S(O)₂-heterocyclyl, —S(O)₂-heteroaryl, —C₁-C₆ alkyl-O—C(O)—C₁-C₆ alkyl, wherein each of the aryl, heterocyclyl, and heteroaryl is independently optionally substituted with 1-3 groups selected from —H, —NO₂, —CN, chloro, bromo, fluoro, iodo, C₁-C₆ alkyl, mono- to per-halogenated-C₁-C₆ alkyl, C₁-C₆ alkoxy, —NH₂, mono- or di-(C₁-C₆ alkyl) amino, mono- to per-halogenated C₁-C₆ alkoxy, and —C(O)—OR₉;
R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—R₉, —C(O)—OR₉, mono- to per-halogenated C₁-C₆ alkoxy, or mono- to per-halogenated C₁-C₆ alkyl; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—R₉, or —C(O)—OR₉;
R₅ is —H, halo, —NO₂, —CN, C₁-C₆ alkyl, C₁-C₆ alkoxy, heterocyclyl-S(O)₂-heteroaryl, aryl-S—C₁-C₆ alkyl, mono- to per-halogenated C₁-C₆ alkyl, —NH₂, mono- or di-(C₁-C₆ alkyl) amino, —C(O)—R₉, —C(O)—OR₉, —N(R₉)—C(O)R₉, —O-(halo C₁-C₆ alkyl), —O-Z; or
R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉; and
R₆ and R₇ are independently —H, —NO₂, —CN, halo, C₁-C₆ alkyl, C₁-C₆ alkoxy optionally substituted with aryl or heteroaryl, mono- to per-halogenated C₁-C₆ alkyl, —NH₂, mono- to per-halogenated C₁-C₆ alkoxy, mono- or di-(C₁-C₆ alkyl) amino, —C(O)—OR₉, aryl, or heteroaryl;
(B):
provided that the compound is not one in which
(C):
W is —O—, and
a) R₁, R₂, R₃, R₄, R₆ and R₇ are —H, and L is —SO₂N(H)— attached at the 4 position of the benzoxadiazolyl ring, and R₅ is —C(O)—OCH₃ or —NH—C(O)CH₃; or —OCF₃;
b) R₁, R₂, R₆ and R₇ are —H, and L is —SO₂N(H)— attached at the 4 position of the benzoxadiazolyl ring, and R₃, R₄ and R₅ are —F;
c) R₂, R₅, R₆ and R₇ are —H, and L is —S— attached at the 4 position of the benzoxadiazolyl ring, and R₁ is —NO₂ and R₃ and R₄ together with the carbon atoms to which they are attached form a pyridinyl group;
d) R₂-R₇ are —H, and L is —S— attached at the 4 position of the benzoxadiazolyl ring, and R₁ is —H or NO₂;
e) R₂-R₇ are —H, and L is —S— or —SO₂N(H)— attached at the 4 position of the benzoxadiazolyl ring, and R₁ is —NH₂;
f) R₂-R₆ are —H, R₇ is —C(O)OH, and L is —S— attached at the 4 position of the benzoxadiazolyl ring, and R₁ is —NO₂;
g) R₁ is —NO₂, L is —S— attached at the 4 or 6 position of the benzoxadiazolyl ring, and A₂, A₃, A₄, A₅, A₆ and A₇ are carbon, and R₂, R₃, 4, R₅, R₆ and R₇ are —H;
h) R₂-R₇ are —H, and L is —SO₂N(H)— attached at the 4 position of the benzoxadiazolyl ring, and R₁ is —F or —N(CH₃)₂; or
i) R₂, R₃, R₄ and R₇ are —H, R₁ is —NO₂, R₆ is —Cl, and L is —S— attached at the 4 position of the benzoxadiazolyl ring, and R₅ is —CH₃, —O—CH₃ or —Br.

The invention also comprises compounds according to Formula I, above, of Formula Ia: 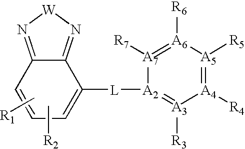
and pharmaceutically acceptable salts, hydrates, solvates, polymorphs, atrophisomers, N-oxides, and prodrugs thereof.

The invention also provides for compounds according to Formula I having the structure 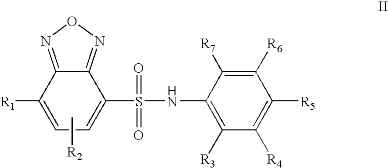
and pharmaceutically acceptable salts, hydrates, solvates, polymorphs, atrophisomers, N-oxides, and prodrugs thereof.

In one embodiment, the compounds of Formula II are of Formula IIa: 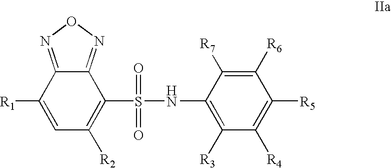
and pharmaceutically acceptable salts, hydrates, solvates, polymorphs, atrophisomers, N-oxides, and prodrugs thereof, wherein
R₁ and R₂ are independently —H, —NO₂, —OH, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN;
R₈ is —H, C₁-C₆ alkyl, or C₁-C₆ alkoxy; R₉ is —H or C₁-C₆ alkyl;
Z is aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉;
   R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—OR₉, or mono- to per-halogenated C₁-C₆ alkyl; or
   R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉;
   R₅ is —H, halo, —NO₂, —CN, C₁-C₆ alkyl, C₁-C₆ alkoxy, mono- to per-halogenated C₁-C₆ alkyl, —NH₂, mono- or di-(C₁-C₆ alkyl) amino, C(O)—OR₉, —N(R₉)—C(O)R₉, or —O-(halo C₁-C₆ alkyl); or
   R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉; and
   R₆ and R₇ are independently —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, aryl, or heteroaryl.

The invention also provides for compounds according to Formula I having the structure 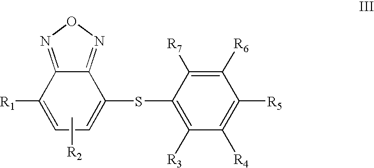
and pharmaceutically acceptable salts, hydrates, solvates, polymorphs, atrophisomers, N-oxides, and prodrugs thereof.

In one embodiment, the compounds of Formula III are of Formula IIa: 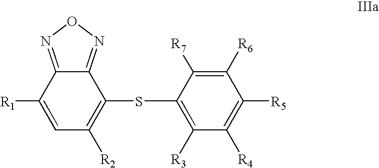
and pharmaceutically acceptable salts, hydrates, solvates, polymorphs, atrophisomers, N-oxides, and prodrugs thereof, wherein
R₁ and R₂ are independently —H, —NO₂, —OH, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN;
R₈ is —H, C₁-C₆ alkyl, or C₁-C₆ alkoxy;
R₉ is —H or C₁-C₆ alkyl;
Z is aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉;
R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—OR₉, or mono- to per-halogenated C₁-C₆ alkyl; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉;
R₅ is —H, halo, —NO₂, —CN, C₁-C₆ alkyl, C₁-C₆ alkoxy, mono- to per-halogenated C₁-C₆ alkyl, —NH₂, mono- or di-(C₁-C₆ alkyl) amino, C(O)—OR₉, —N(R₉)—C(O)R₉, or —O-(halo C₁-C₆ alkyl); or
R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉; and
R₆ and R₇ are independently —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, aryl, or heteroaryl;
provided that:
when R₁ is —NO₂, then R₂, R₃, R₄, R₅, R₆ and R₇ are not —H.

The invention also provides for compounds according to Formula I having the structure 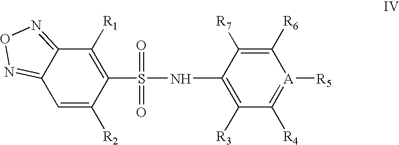
and pharmaceutically acceptable salts, hydrates, solvates, polymorphs, atrophisomers, N-oxides, and prodrugs thereof, wherein
R₁ and R₂ are independently —H, C₁-C₄ alkyl, or C₁-C₄ alkoxy;
A is carbon or nitrogen provided that when A is nitrogen R₅ is absent;
R₃ and R₄ are independently —H, halo, C₁-C₄ alkyl, or C₁-C₄ alkoxy; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, halo, C₁-C₄ alkyl, or C₁-C₄ alkoxy;
R₅ is —H, halo, C₁-C₄ alkyl, or C₁-C₄ alkoxy;
R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, halo, C₁-C₆ alkyl, C₁-C₆ alkoxy; and
R₆ and R₇ are independently —H, C₁-C₄ alkyl, or C₁-C₄ alkoxy.

In one embodiment, the compounds according to Formula II are compounds wherein R₃, R₄, R₆ and R₇ are —H, R₅ is —C(O)—OCH₃, and R₁ and R₂ are independently —H or N(R₈)-Z, wherein Z is substituted with —C(O)—OR₉. Preferably, R₁ is H and R₂ is N(R₈)-Z, wherein Z is substituted with —C(O)—OR₉. Also preferred are compound wherein R₂ is H and R₁ is N(R₈)-Z substituted with —C(O)—OR₉. More preferably, the compounds are those wherein Z is aryl, R₈ is —H and R₉ is C₁-C₂ alkyl.

In another embodiment, the compound according to Formula II are compounds wherein R₁, R₂, R₅, R₆, and R₇ are —H, R₃ and R₄ are independently —H, C₁-C₂ alkoxy, or R₃ and R₄ together with the carbon atoms to which they are attached form a heteroaryl group. Preferably, R₄ is —H and R₃ is C₁-C₂ alkoxy. Also preferred are compound wherein R₃ and R₄ together with the carbon atoms to which they are attached form a heteroaryl group.

In yet another embodiment, the compounds of Formula II are compound wherein R₁, R₂, R₄, R₅, and R₇ are —H, R₃ is C₁-C₂ alkyl, and R₆ is halo.

In another embodiment, the compounds according to Formula IV are compounds wherein R₁ to R₄, R₆ and R₇ are —H, R₅ is absent and A is nitrogen.

In another embodiment, the compounds according to Formula's I and II are those in which R₁ is —N(R₉)Z.

The invention also provides for compounds according Formula V 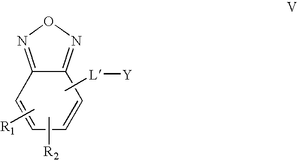
and pharmaceutically acceptable salts, hydrates, solvates, polymorphs, atrophisomers, N-oxides, and prodrugs thereof, wherein
L′ is —SO₂-aryl, —SO₂-heteroaryl, —SO₂-heterocyclyl, —N(H)-heterocyclyl, —N(H)-heteroaryl, —N(H)C(O)-heterocyclyl, —N(H)C(O)-heteroaryl, C₃-C₆-cycloalkenyl, —S-heteroaryl, aryl, heteroaryl or heterocyclyl, wherein each of the aryl, heterocyclyl and heteroaryl is optionally substituted with 1 to 3 groups selected from —H, —OH, —S-heteroaryl, —S(O)₂-heteroaryl, heteroaryl, —O-heteroaryl, C₁-C₆ alkyl, C₁-C₆ alkoxy, C₁-C₆ alkyl, —C(O)—OR₉, —N(R₉)—C(O)R₉, —O—(C₁-C₆ alkyl), —NH₂, mono- or di-(C₁-C₆ alkyl) amino, —NO₂, halo, or —CN, or spiro-substituted with heterocyclyl or heteroaryl, each of which is optionally substituted with —H, OH, halo, C₁-C₆ alkyl, C₁-C₆ alkoxy, or oxo;
R₁ and R₂ are independently —H, —NO₂, —OH, —CN, halo, C₁-C₆ alkyl, C₁-C₆ alkoxy, mono- to per-halogenated C₁-C₆ alkyl, heterocyclyl, heterocyclyl-O-aryl, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN; or
R₁ and R₂ together with the carbon atoms to which they are attached form an aryl group optionally substituted with 1 to 3 groups selected from —OH, halo, C₁-C₆-alkyl, C₁-C₆-alkoxy, —NH₂. and —NO₂;
Y is —H, —NO₂, —OH, —CN, halo, C₁-C₆ alkyl, C₁-C₆ alkoxy, —NH₂, mono- or di-(C₁-C₆ alkyl) amino, —C(O)—OR₉, —C(O)R₉, —N(R₈)-Z, C₁-C₆ alkyl-aryl, C₁-C₆ alkyl-heterocyclyl, C₁-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, —O-aryl, aryl, heterocyclyl, heteroaryl, or -Z₁-S(O)₂-Z₂ where Z, and Z₂ are independently aryl or heteroaryl, wherein each of the aryl, heterocyclyl and heteroaryl is optionally substituted with 1 to 3 groups selected from —H, C₁-C₆ alkyl, C₁-C₆ alkoxy, mono- to per-halogenated C₁-C₆ alkyl, —C(O)—OR₉, —N(R₉)—C(O)R₉, —O-(halo C₁-C₆ alkyl), —NH₂, mono- or di-(C₁-C₆ alkyl) amino, —NO₂, halo, or —CN;
R₈ is —H, C₁-C₆ alkyl, or C₁-C₆ alkoxy;
R₉ is —H, C₁-C₆ alkyl or C₃-C₆ cycloalkyl; and
Z is —C₁-C₆ alkyl-O—C(O)—C₁-C₆ alkyl, aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

In one embodiment according to Formula V, the invention comprises compounds of structural formula Va: 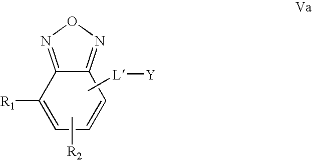
and pharmaceutically acceptable salts, hydrates, solvates, polymorphs, atrophisomers, N-oxides, and prodrugs thereof.

In one embodiment according to Formula V, the invention provides for compounds of formula VI 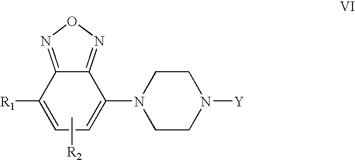
and pharmaceutically acceptable salt, hydrates, solvates, polymorphs, atrophisomers, N-oxides, and prodrugs thereof, wherein
R₁ and R₂ are independently —H, —NO₂, —OH, —CN, halo, C₁-C₆ alkyl, C₁-C₆ alkoxy, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, —NH₂, mono- or di-(C₁-C₆ alkyl) amino, —NO₂, halo, or —CN;
Y is —H, —NO₂, —OH, —CN, halo, C₁-C₆ alkyl, C₁-C₆ alkoxy, —NH₂, mono- or di-(C₁-C₆ alkyl) amino, —C(O)—OR₉, —C(O)R₉, —N(R₈)-Z, C₀-C₆ alkyl-aryl, C₀-C₆ alkyl-heterocyclyl, C₀-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, aryl, heterocyclyl or heteroaryl, wherein each of the aryl, heterocyclyl and heteroaryl is optionally substituted with 1 to 3 groups selected from —H, C₁-C₆ alkyl, C₁-C₆ alkoxy, mono- to per-halogenated C₁-C₆ alkyl, —C(O)—OR₉, —N(R₉)—C(O)R₉, —O-(halo C₁-C₆ alkyl), —NH₂, mono- or di-(C₁-C₆ alkyl) amino, —NO₂, halo, or —CN;
R₈ is —H, C₁-C₆ alkyl, or C₁-C₆ alkoxy;
R₉ is —H, C₁-C₆ alkyl or C₃-C₆ cycloalkyl; and
Z is aryl, heteroaryl, —C(O)-aryl, or —C(O)-heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

In one embodiment according to formula VI, the invention provides for compound wherein R₁ and R₂ are independently —H, —NO₂, —OH, —CN, halo, C₁-C₆ alkyl, C₁-C₆ alkoxy, or —NH₂, and Y is —H, C₁-C₆ alkyl, —C(O)—OR₉, —C(O)R₉, C₀-C₆ alkyl-aryl, C₀-C₆ alkyl-heterocyclyl, C₀-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, wherein each of the aryl, heterocyclyl and heteroaryl is optionally substituted with 1 to 3 groups selected from halo-C₁-C₆ alkyl, halo and —CN.

In one other embodiment according to formula VI, the invention provides for compound wherein R₂ is H, R₁ is —H or —NO₂ and Y is —H, C₁-C₆ alkyl, —C(O)—OR₉, —C(O)R₉, C₀-C₆ alkyl-aryl, C₀-C₆ alkyl-heterocyclyl, C₀-C₆ alkyl-heteroaryl, C₀-C₆ alkyl-C(O)-heterocyclyl, wherein each of the aryl, heterocyclyl and heteroaryl is optionally substituted with 1 to 3 groups selected from halo, halo-C₁-C₆ alkyl and —CN. In one aspect, R₁ is NO₂, and Y is H, —C(O)—OR₉, C₀-C₆ alkyl-heteroaryl, C₀-C₆ alkyl-heterocyclyl, C₀-C₆ alkyl-aryl or C₁-C₆ alkyl, wherein each of the aryl and heteroaryl is optionally substituted with 1 to 3 groups selected from halo, halo-C₁-C₆ alkyl and —CN. In another aspect, Y is —C(O)—OR₉ wherein R₉ is C₁-C₆ alkyl. In still another aspect, Y is C₀-C₆ alkyl-heteroaryl optionally substituted with —CN or —CF₃. Preferably, Y is pyrazine, pyrimidine, or pyridine wherein the pyridine is optionally substituted with —CN or —CF₃. In yet another aspect, Y is C₀-C₆ alkyl-aryl optionally substituted with 1 or 2 groups that are selected from halo and halo-C₁-C₆ alkyl. Preferably, Y is phenyl optionally substituted with 1 or 2 groups that are selected from chloro and trifluoromethyl. In still another aspect, Y is C₁-C₄ alkyl or H. In one other aspect, Y is C₀-C₆ alkyl-heterocyclyl.

In one other embodiment according to Formula V, the invention provides for compounds of formula VII 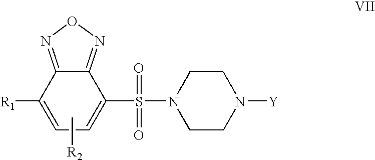
and pharmaceutically acceptable salts, hydrates, solvates, polymorphs, atrophisomers, N-oxides, and prodrugs thereof, wherein
R₁ and R₂ are independently —H, —NO₂, —OH, —CN, halo, C₁-C₆ alkyl, C₁-C₆ alkoxy, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, —NH₂, mono- or di-(C₁-C₆ alkyl) amino, —NO₂, halo, or —CN;
Y is —H, —NO₂, —OH, —CN, halo, C₁-C₆ alkyl, C₁-C₆ alkoxy, —NH₂, mono- or di-(C₁-C₆ alkyl) amino, —C(O)—OR₉, —C(O)R₉, —N(R₈)-Z, C₁-C₆ alkyl-aryl, C₁-C₆ alkyl-heterocyclyl, C₁-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, aryl, heterocyclyl or heteroaryl, wherein each of the aryl, heterocyclyl and heteroaryl is optionally substituted with 1 to 3 groups selected from —H, C₁-C₆ alkyl, C₁-C₆ alkoxy, halo-C₁-C₆ alkyl, —C(O)—OR₉, —N(R₉)—C(O)R₉, —O-(halo C₁-C₆ alkyl), —NH₂, mono- or di-(C₁-C₆ alkyl) amino, —NO₂, halo, or —CN;
R₈ is —H, C₁-C₆ alkyl, or C₁-C₆ alkoxy; R₉ is —H, C₁-C₆ alkyl or C₃-C₆ cycloalkyl; and
Z is aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

In one other embodiment according to formula VII, the invention provides for compound wherein R₁ and R₂ are independently —H, —NO₂, —OH, —CN, halo, C₁-C₆ alkyl, C₁-C₆ alkoxy, or —NH₂, and Y is —H, —C(O)R₉, C₁-C₆ alkyl-aryl, C₁-C₆ alkyl-heterocyclyl, C₁-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, aryl, heterocyclyl or heteroaryl, wherein each of the aryl, heterocyclyl and heteroaryl is optionally substituted with 1 to 3 groups selected from halo-C₁-C₆ alkyl, halo and —CN.

In another embodiment according to formula VII, the invention provides for compound wherein R₁ and R₂ are H, Y is —C(O)R₉, C₁-C₆ alkyl-heterocyclyl, C₀-C₆ alkyl-C(O)-heterocyclyl, aryl or heteroaryl, wherein each of the aryl and heteroaryl is optionally substituted with 1 to 3 groups selected from halo-C₁-C₆ alkyl, halo and —CN. In one aspect, Y is heteroaryl optionally substituted with 1 to 3 groups selected from halo-C₁-C₆ alkyl and —CN. Preferably, Y is pyridine, pyrimidine or pyrazine optionally substituted with 1 or 2 groups selected from —CN and —CF₃—In another aspect, Y is aryl. Preferably, Y is phenyl optionally substituted with 1 or 2 groups selected from —CF₃ and chloro. In still another aspect, Y is C₁-C₆ alkyl-heterocyclyl. In yet another aspect, Y is —C(O)R₉ wherein R₉ is C₁-C₄-alkyl. In still one more aspect, Y is C₀-C₆ alkyl-C(O)-heterocyclyl. Preferably Y is C₁-C₄ alkyl-C(O)-heterocyclyl.

The invention also provides for compounds of formula VIII 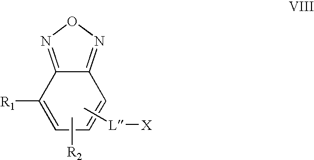
and pharmaceutically acceptable salts, hydrates, solvates, polymorphs, atrophisomers, N-oxides, and prodrugs thereof, wherein
L″ is —S— or —SO₂N(R₉)—C₀-C₆ alkyl;
X is C₁-C₆ alkyl, halo, C₃-C₆ cycloalkyl, C₁-C₆ alkyl-C₁-C₆ alkoxy, C₁-C₆ alkoxy, mono- or di-(C₁-C₆ alkyl) amino, aryl, heterocyclyl, C₃-C₆ cycloalkyl, or heteroaryl, wherein each of the alkyl, aryl, heterocyclyl and heteroaryl is optionally substituted with 1 to 3 groups selected from —H, oxo, —O—C₀-C₆ alkyl-aryl, —O—SO₂-heteroaryl wherein the heteroaryl is optionally substituted with halo, —OH, C₁-C₆ alkyl, C₁-C₆ alkoxy, —NH₂, mono- or di-(C₁-C₆ alkyl) amino, —NO₂, halo, or —CN;
R₁ and R₂ are independently —H, —NO₂, —OH, —CN, halo, C₁-C₆ alkyl, C₁-C₆ alkoxy, —NH₂, —S-heteroaryl, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, —NH₂, mono- or di-(C₁-C₆ alkyl) amino, —NO₂, halo, or —CN;
R₈ is —H, C₁-C₆ alkyl, or C₁-C₆ alkoxy;
R₉ is —H or C₁-C₆ alkyl; and
Z is —C₁-C₆ alkyl-O—C(O)—C₁-C₆ alkyl, aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

In one embodiment according to formula VIII, the invention provides for compound wherein R₁ and R₂ are independently —H, —NO₂, —OH, —CN, halo, C₁-C₆ alkyl, C₁-C₆ alkoxy or —NH₂, L″ is —SO₂N(R₉)—C₀-C₆ alkyl, and X is C₁-C₆ alkoxy, C₃-C₆ cycloalkyl, heterocyclyl, or halo. In one aspect, R₁ and R₂ are H, L″ is —SO₂N(H)—C₁-C₂ alkyl, and X is C₁-C₄ alkoxy, C₃-C₆ cycloalkyl, heterocyclyl. In another aspect, R₁ and R₂ are —H, L″ is —SO₂N(R₉)—C₀-C₆ alkyl, and X is mono- or di-(C₁-C₆ alkyl) amino. Preferably, L″ is —SO₂N(R₉)—C₁-C₄ alkyl and X is mono-(C₁-C₄ alkyl) amino wherein R₉ is —C₁-C₄ alkyl.

The invention also provides compounds of formulas IXa and IXb: 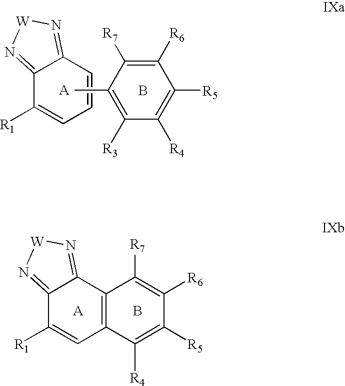
and pharmaceutically acceptable salts, hydrates, solvates, polymorphs, atrophisomers, N-oxides, and prodrugs thereof, wherein
W is —O— or —S(O)₀₋₂;
R₁, is —H, —NO₂, —OH, —CN, halo, C₁-C₆ alkyl, C₁-C₆ alkoxy, mono- to per-halogenated C₁-C₆ alkyl, —NH₂, mono- or di-(C₁-C₆ alkyl) amino, or aryl, wherein the aryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN;
R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—OR₉, or mono- to per-halogenated C₁-C₆ alkyl; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, or mono- or di-(C₁-C₆ alkyl) amino;
R₅ is —H, halo, —NO₂, —CN, C₁-C₆ alkyl, C₁-C₆ alkoxy, —S—C₁-C₆ alkyl, mono- to per-halogenated C₁-C₆ alkyl, —NH₂, mono- or di-(C₁-C₆ alkyl) amino; and
R₆ and R₇ are independently —H, —NO₂, —CN, halo, C₁-C₆ alkyl, C₁-C₆ alkoxy, mono- to per-halogenated C₁-C₆ alkyl, —NH₂, or mono- or di-(C₁-C₆ alkyl) amino.

In the second aspect, the invention provides for pharmaceutical compositions comprising, together with a pharmaceutically acceptable carrier, diluent, or excipient, a compound of according to any one of Formulae I to IXb as described above.

In another embodiment of this second aspect, the invention provides for pharmaceutical compositions comprising, together with a pharmaceutically acceptable carrier, diluent, or excipient, a compound as described in part (C) of formula I in paragraph [0028], except for subpart (f). In one embodiment, compounds of the second aspect of the invention are those of formula I: 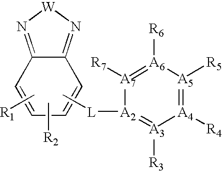
and pharmaceutically acceptable salts, hydrates, solvates, polymorphs, atrophisomers, N-oxides, and prodrugs thereof, wherein
W is —O—;
A₂, A₃, A₄, A₅, A₆ and A₇ are independently carbon or nitrogen provided that when any one of A₂, A₃, A₄, A₅, A₆ and A₇ is nitrogen the R group attached to it is absent, or when any one of A₂, A₃, A₄, A₅, A₆ and A₇ is nitrogen and the R moiety attached to it is present, then the nitrogen has a positive charge and is a quaternary ammonium;
wherein R₁-R₇ and L are selected from the following definitions:
a) R₁, R₂, R₃, R₄, R₆ and R₇ are —H, and L is —SO₂N(H)— attached at the 4 position of the benzoxadiazolyl ring, and R₅ is —C(O)—OCH₃ or —NH—C(O)CH₃; or —OCF₃;
b) R₁, R₂, R₆ and R₇ are —H, and L is —SO₂N(H)— attached at the 4 position of the benzoxadiazolyl ring, and R₃, R₄ and R₅ are —F;
c) R₂, R₅, R₆ and R₇ are —H, and L is —S— attached at the 4 position of the benzoxadiazolyl ring, and R₁ is —NO₂ and R₃ and R₄ together with the carbon atoms to which they are attached form a pyridinyl group;
d) R₂-R₇ are —H, and L is —S— attached at the 4 position of the benzoxadiazolyl ring, and R₁ is —H or NO₂;
e) R₂-R₇ are —H, and L is —S— or —SO₂N(H)— attached at the 4 position of the benzoxadiazolyl ring, and R₁ is —NH₂;
f) R₁ is —NO₂, L is —S— attached at the 4 or 6 position of the benzoxadiazolyl ring, and A₂, A₃, A₄, A₅, A₆ and A₇ are carbon, and R₂, R₃, R₄, R₅, R₆ and R₇ are —H;
g) R₂-R₇ are —H, and L is —SO₂N(H)— attached at the 4 position of the benzoxadiazolyl ring, and R₁ is —F or —N(CH₃)₂; and
h) R₂, R₃, R₄ and R₇ are —H, R₁ is —NO₂, R₆ is —Cl, and L is —S— attached at the 4 position of the benzoxadiazolyl ring, and R₅ is —CH₃, —O—CH₃ or —Br.

The compounds according to the first aspect of the invention are also useful as general ubiquitin ligase inhibitors. For example, the compounds of the invention can be used as inhibitors of enzymes that exhibit ligase activity, including but not limited to TRAF6, APC and E3 enzymes. The compounds of the invention are also useful for regulating or inhibiting pathways in diseases and conditions that involve ubiquitin conjugation and deconjugation such as cancers, inflammation, metabolism, viral diseases and central nervous system disorders. For example, the compounds of the invention can be used to regulate or inhibit the products of genes that encode ubiquitin or ubiquitin-like enzymes described in Wong et al. (DDT 8 (16), 746-754 (2003)), which is incorporated by reference in its entirety.

In the third aspect, the invention provides methods of inhibiting ubiquitination in a cell comprising contacting the cell in which inhibition of ubiquitination is desired with a compound according to Formulae I to IXb or a pharmaceutical composition according to the second aspect of the invention. The compounds and formulations of the invention can inhibit ubiquitination in cells derived from animals, particularly, mammalian cells. The compounds and formulations of the invention can also be used to inhibit the ubiquitin ligase activity of TRAF6.

In the fourth aspect, the invention provides for methods of treating cell proliferative diseases or conditions comprising administering to a patient an effective amount of a compound or pharmaceutical composition according to the invention. Cell proliferative diseases or conditions include, but are not limited to, psoriasis, keloid scarring, and cancers, such as cancers of the breast, immune system, bone, nervous system, brain, blood, lymphatic system, and skin. Particularly, the compounds and pharmaceutical compositions of the invention are useful for treating cell proliferative diseases or conditions that involve TRAF6.

In the fifth aspect, the invention provides for methods of inhibiting TRAF6 comprising administering to a patient an effective amount of a compound or pharmaceutical composition according to the invention. Particularly, the compounds and pharmaceutical compositions are useful for treating conditions or diseases that involve TRAF6 such as those related to cancer, inflammation, adaptive immunity, innate immunity, bone metabolism, LPS-induced angiogenesis, osteoporosis, osteopinneal diseases, lymph node development, mammary gland development, skin development, and central nervous system development.

The compounds according to the fifth aspect of the invention are also useful as general ubiquitin ligase inhibitors. For example, the compounds of the invention can be used as inhibitors of E3 enzymes that contain HECT and RING finger domains, Mdm2 with RING fingers and variants, and U-box-containing proteins. Accordingly, the compounds of the invention are useful as protein modulators, immunologic agents anti-inflammatory agents, anti-osteoporosis agents, anti-viral agents, for example, inhibitors of variola viruses such as smallpox, HIV and related conditions, human papillomavirus, HSV, adenovirus, coxsackie virus, HCMV, KSHV, EBV, paramyxovirus, myxomavirus, ebola, retrovirus, and rhabdovirus, anti-protozoan agents, for example, inhibitors of the malaria parasite. The compounds of the invention are also useful as oncologic and anti-proliferative agents that inhibit aberrant cell growth, cancers, restenosis, psoriasis, and neoplastic cell proliferation.

Inhibition of TRAF6 activity by the compounds and pharmaceutical compositions of the invention provides ways to regulate the expression of genes involved many biological processes. Such processes include but are not limited to bone metabolism and signal transduction pathways that involve IL-1, CD40, RANKL, LPS, IL-17, and LMP1. For example, the compounds and pharmaceutical compositions of the invention can be used to regulate the activities of transcription factors that activate the expression of genes, such as NF-kB and AP-1. The compounds and pharmaceutical compositions can also be used to regulate the activities of kinases, such as MAP kinases, JNK/SAPK, ERK, p38, IkB kinase and Src-family tyrosine kinases.

Inhibition of TRAF6 serves as a therapeutic target for inflammatory and autoimmune diseases. For example, TRAF6 plays a critical regulator role of the TNFR/IL-1R/TLR signal transduction pathways and can serve as a broad anti-inflammation target for inflammatory diseases such as RA, COPD, IBD. Further TRAF6 can also be a useful therapeutic target for treating autoimmune diseases, such as graft rejection because of its regulator role in the CD40 signaling cascade. TRAF6 is also a target for treating bone-destructive diseases, such as osteoporosis and rheumatoid arthritis because TRAF6 plays a critical regulator role of the RANK signal transduction that mediate osteoclast activation and function. Similarly, TRAF6 may also serve as a novel allergic and infective disease target for treating bacterial sepsis and associated systemic inflammation because TRAF6 plays critical mediator roles in the TLR signal transduction which is involved in the interaction between dentritic cells, T lymphocytes and mast cells.

Inhibition of TRAF6 may also serve as a therapeutic target in diseases and conditions that involve non-degradative ubiquitination. For example, TRAF6 acts as an E3 ubiquitin ligase that mediates kinase activation by K-63 linked, non-degradative ubiquitination. Inhibiting TRAF6's E3 ligase activity may provide novel anti-inflammation therapeutics.

Some useful compounds according to one aspect of the invention are given in the following table and can be used in pharmaceutical compositions.

TABLE 1

| Cpd | | |
|---|---|---|
| No. | Name | Structure |
| 8 | methyl 4-{[(7-chloro-2,1,3-benzoxadiazol- 4-yl)sulfonyl]amino}benzoate | 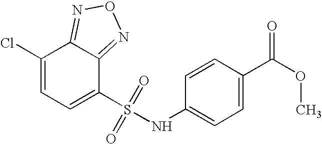 |
| 9 | methyl 4-{[(7-{[4- (methoxycarbonyl)phenyl]amino}-2,1,3- benzoxadiazol-4- yl)sulfonyl]amino}benzoate | 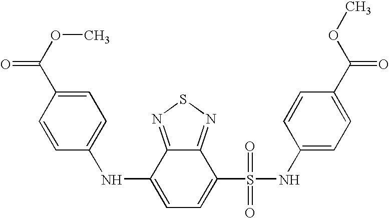 |
| 10 | methyl 4-{[(5-{[4- (methoxycarbonyl)phenyl]amino}-2,1,3- benzoxadiazol-4- yl)sulfonyl]amino}benzoate | 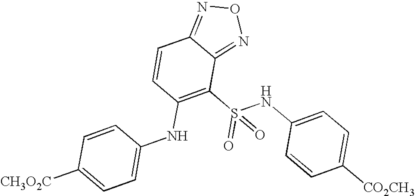 |
| 11 | N-(2-methoxyphenyl)-2,1,3- benzoxadiazole-4-sulfonamide | 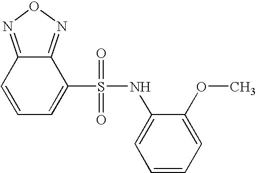 |
| 12 | N-isoquinolin-5-yl-2,1,3-benzoxadiazole- 4-sulfonamide | 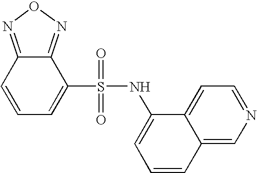 |
| 13 | N-(5-fluoro-2-methylphenyl)-2,1,3- benzoxadiazole-4-sulfonamide | 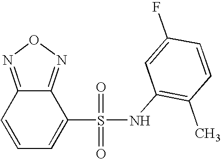 |
| 14 | N-(3-chloro-4-fluorophenyl)-2,1,3- benzoxadiazole-4-sulfonamide | 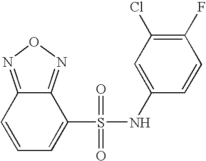 |
| 15 | N-(2,6-difluorophenyl)-2,1,3- benzoxadiazole-4-sulfonamide | 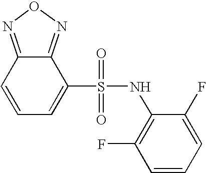 |
| 16 | N-(2,4,6-trifluorophenyl)-2,1,3- benzoxadiazole-4-sulfonamide | 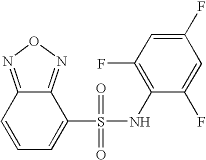 |
| 17 | N-[2-methyl-3-(trifluoromethyl)phenyl]- 2,1,3-benzoxadiazole-4-sulfonamide | 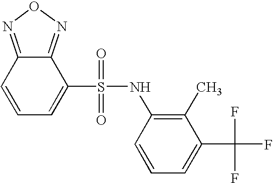 |
| 18 | N-(4-iodo-3-methylphenyl)-2,1,3- benzoxadiazole-4-sulfonamide | 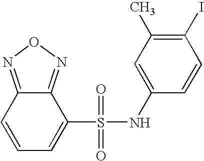 |
| 19 | N-(4-fluoro-2-methylphenyl)-2,1,3- benzoxadiazole-4-sulfonamide | 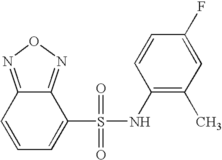 |
| 20 | N-(2,3-difluorophenyl)-2,1,3- benzoxadiazole-4-sulfonamide | 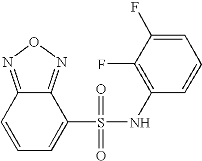 |
| 21 | N-(2,5-dimethylphenyl)-2,1,3- benzoxadiazole-4-sulfonamide | 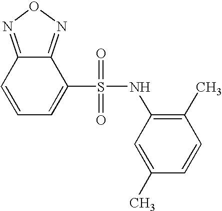 |
| 22 | N-(3-fluorophenyl)-2,1,3-benzoxadiazole- 4-sulfonamide | 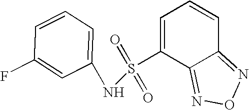 |
| 23 | N-(3,4-difluorophenyl)-2,1,3- benzoxadiazole-4-sulfonamide | 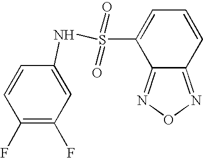 |
| 24 | N-(2-methyl-1H-indol-5-yl)-2,1,3- benzoxadiazole-4-sulfonamide | 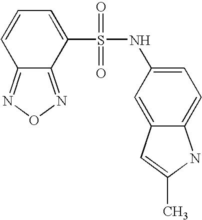 |
| 25 | N-mesityl-2,1,3-benzoxadiazole-4- sulfonamide | 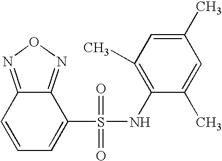 |
| 26 | N-pyridin-4-yl-2,1,3-benzoxadiazole-5- sulfonamide | 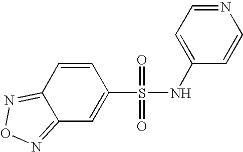 |
| 27 | N-(2,5-difluorophenyl)-2,1,3- benzoxadiazole-4-sulfonamide | 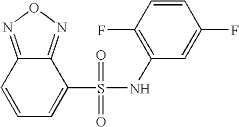 |
| 43 | N-(3-chlorophenyl)-2,1,3-benzoxadiazole- 4-sulfonamide | 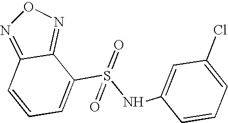 |
| 46 | N-benzyl-2,1,3-benzoxadiazole-5- sulfonamide | 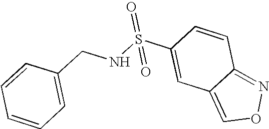 |
| 49 | N-(pyridin-2-ylmethyl)-2,1,3- benzoxadiazole-4-sulfonamide | 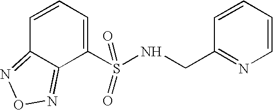 |
| 50 | N-(2-methoxyethyl)-2,1,3-benzoxadiazole- 4-sulfonamide | 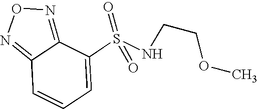 |
| 51 | N-(4-methoxyphenyl)-2,1,3- benzoxadiazole-4-sulfonamide | 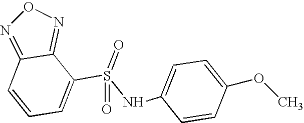 |
| 52 | N-(3,5-dichlorophenyl)-2,1,3- benzoxadiazole-4-sulfonamide | 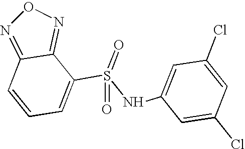 |
| 53 | N-(cyclohexylmethyl)-2,1,3- benzoxadiazole-4-sulfonamide | 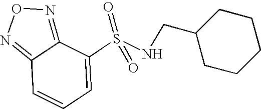 |
| 54 | N-(2-chloro-4-methylphenyl)-2,1,3- benzoxadiazole-4-sulfonamide | 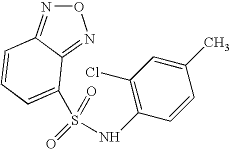 |
| 55 | N-(2-pyrrolidin-1-ylethyl)-2,1,3- benzoxadiazole-4-sulfonamide | 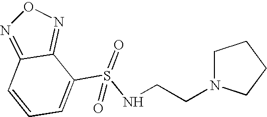 |
| 56 | N-(5-chloro-2-methylphenyl)-2,1,3- benzoxadiazole-4-sulfonamide | 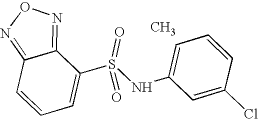 |
| 57 | N-(2-iodophenyl)-2,1,3-benzoxadiazole-4- sulfonamide | 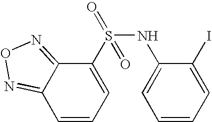 |
| 58 | N-pyridin-2-yl-2,1,3-benzoxadiazole-4- sulfonamide | 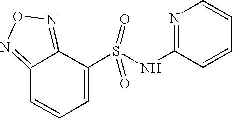 |
| 59 N-(2-fluoroethyl)-2,1,3-benzoxadiazole-4- sulfonamide | 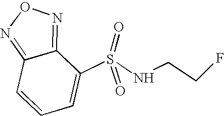 | |
| 60 | N-(2,5-dichlorophenyl)-2,1,3- benzoxadiazole-4-sulfonamide | 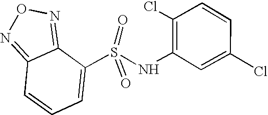 |
| 61 | N-(2-fluoro-5-nitrophenyl)-2,1,3- benzoxadiazole-4-sulfonamide | 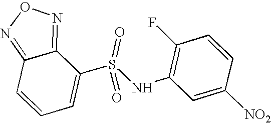 |
| 62 | N-(4-{[3-chloro-5- (trifluoromethyl)pyridin-2-yl]oxy}phenyl)- 2,1,3-benzoxadiazole-4-sulfonamide | 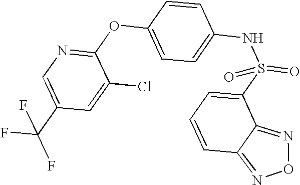 |
| 63 | dimethyl 2-[(2,1,3-benzoxadiazol-4- ylsulfonyl)amino]terephthalate | 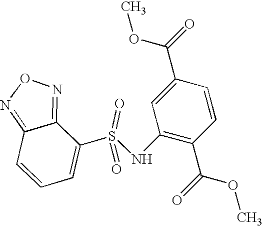 |
| 64 | N-quinolin-8-yl-2,1,3-benzoxadiazole-4- sulfonamide | 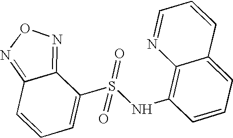 |
| 65 | N-(4-nitrophenyl)-2,1,3-benzoxadiazole-4- sulfonamide | 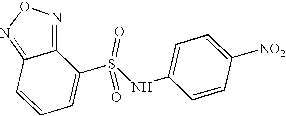 |
| 66 | N-(3-chloro-4-morpholin-4-ylphenyl)- 2,1,3-benzoxadiazole-4-sulfonamide | 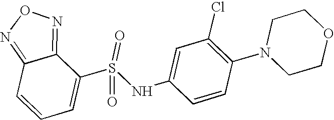 |
| 67 | N[2-(trifluoromethoxy)phenyl]2,1,3- benzoxadiazole-4-sulfonamide | 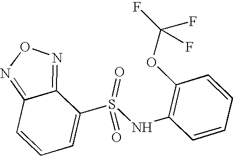 |
| 68 | N-pyridin-4-yl-2,1,3-benzoxadiazole-4- sulfonamide | 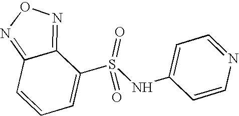 |
| 69 | N-2,1,3-benzothiadiazol-4-yl-4- iodobenzenesulfonamide | 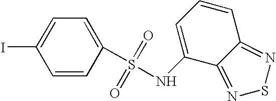 |
| 70 | N-2,1,3-benzothiadiazol-4-yl-3,4- difluorobenzenesulfonamide | 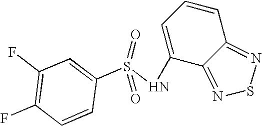 |
| 71 | N-2,1,3-benzothiadiazol-4-yl-3,4,5- trifluorobenzenesulfonamide | 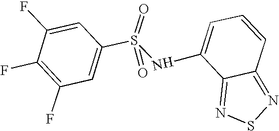 |
| 72 | tert-butyl 4-(7-nitro-2,1,3-benzoxadiazol- 4-yl)piperazine-1-carboxylate | 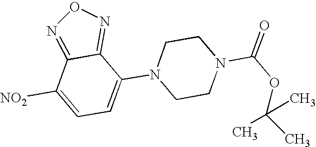 |
| 73 | N-2,1,3-benzothiadiazol-4-yl-3,5-dichloro-4- (2-chloro-4- nitrophenoxy)benzenesulfonamide | 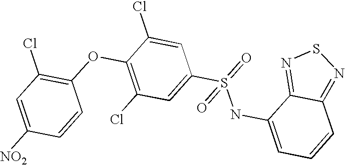 |
| 74 | ethyl 3-[(2,1,3-benzoxadiazol-4- ylsulfonyl)amino]benzoate | 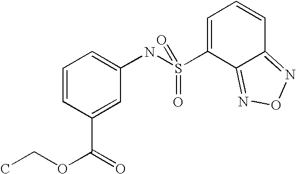 |
| 75 | N-2,1,3-benzothiadiazol-4-yl-2-bromo-4,6- difluorobenzenesulfonamide | 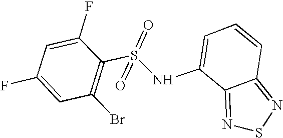 |
| 76 | N-2,1,3-benzothiadiazol-4-yl-3-chloro-4- fluorobenzenesulfonamide | 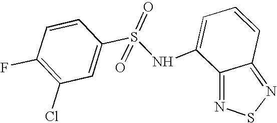 |
| 77 | N-2,1,3-benzothiadiazol-4-yl-2-bromo-4- fluorobenzenesulfonamide | 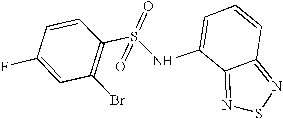 |
| 78 | N-2,1,3-benzoxadiazol-4-yl-4-{[3-chloro- 5-(trifluoromethyl)pyridin-2- yl]oxy}benzenesulfonamide | 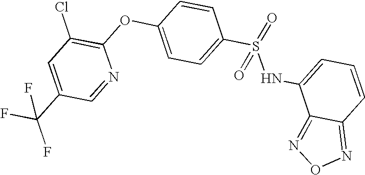 |
| 79 | N-2,1,3-benzoxadiazol-4-yl-3-chloro-4- (2,6-dichloro-4- nitrophenoxy)benzenesulfonamide | 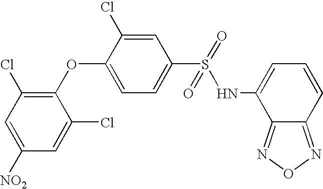 |
| 80 | N-2,1,3-benzoxadiazol-4-yl-4-(pyridin-4- yloxy)benzenesulfonamide | 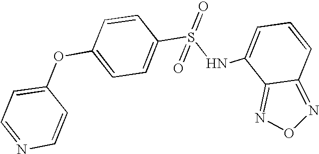 |
| 81 | N-2,1,3-benzoxadiazol-4-yl-4-{[5,7- bis(trifluoromethyl)-1,8-naphthyridin-2- yl]oxy}benzenesulfonamide | 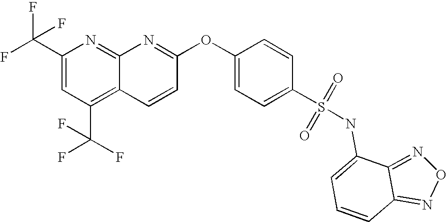 |
| 82 | 4-nitro-7-(4-pyridin-2-ylpiperazin-2-yl)- 2,1,3-benzoxadiazole | 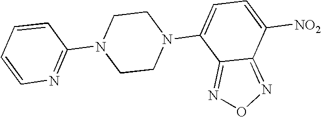 |
| 83 | N-2,1,3-benzoxadiazol-4-yl-3,5-dichloro- 4-hydroxybenzenesulfonamide | 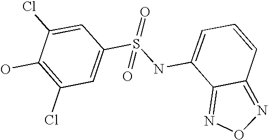 |
| 84 | 4-nitro-7-(4-pyrazin-2-ylpiperazin- 2,1,3-benzoxadiazole | 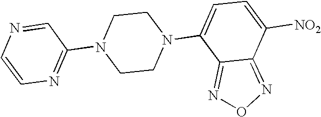 |
| 85 | 4-[4-(2-morpholin-4-ylethyl)piperazin-1- yl]-7-nitro-2,1,3-benzoxadiazole | 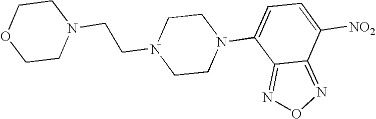 |
| 86 | N-2,1,3-benzoxadiazol-4-yl-4-(pyridin-3- yloxy)benzenesulfonamide | 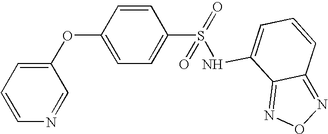 |
| 87 | N-2,1,3-benzoxadiazol-4-yl-5- phenoxypyridine-2-sulfonamide | 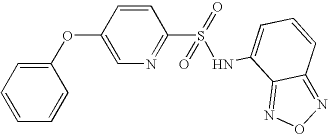 |
| 88 | 4-(4-acetylpiperazin-1-yl)-7-nitro-2,1,3- benzoxadiazole | 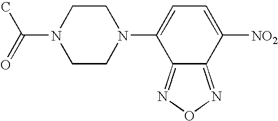 |
| 89 | N-2,1,3-benzothiadiazol-4-yl-4- fluorobenzenesulfonamide | 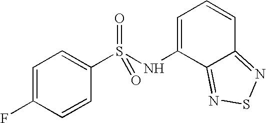 |
| 90 | 4-nitro-7-{4-[4-(trifluoromethyl)pyridin-2- yl]piperazin-1-yl}-2,1,3-benzoxadiazole | 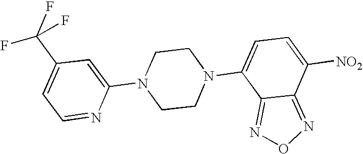 |
| 91 | N-[3-chloro-4-(pyrimidin-2-yloxy)phenyl]- 2,1,3-benzoxadiazole-4-sulfonamide | 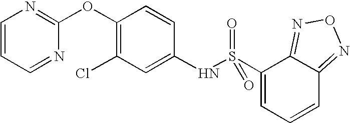 |
| 92 | N-[3-bromo-4-(4-fluorophenoxy)phenyl]- 2,1,3-benzoxadiazole-4-sulfonamide | 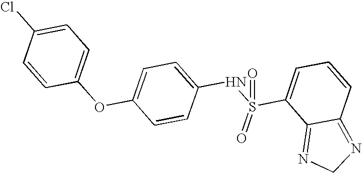 |
| 93 | N-2,1,3-benzoxadiazol-4-yl-4- phenoxybenzenesulfonamide | 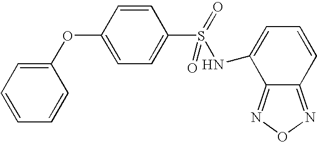 |
| 94 | N-2,1,3-benzothiadiazol-4-yl-4- phenoxybenzenesulfonamide | 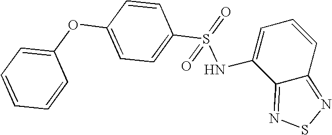 |
| 95 | 4-{[4-(pyrimidin-2-yloxy)piperidin-1- yl]sulfonyl}-2,1,3-benzoxadiazole | 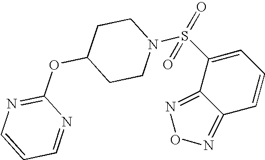 |
| 96 | N-2,1,3-benzothiadiazol-4-yl-3,5-dichloro- 4-hydroxybenzenesulfonamide | 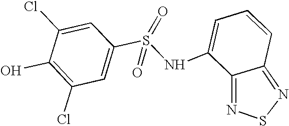 |
| 97 | N-2,1,3-benzothiadiazol-4-yl-3- bromoberizenesulfonamide | 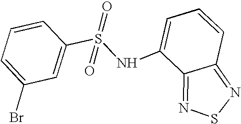 |
| 98 | N-2,1,3-benzothiadiazol-4-yl-4- (bromomethyl)benzenesulfonamide | 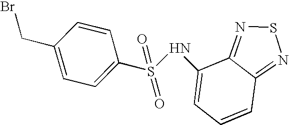 |
| 99 | N-2,1,3-benzothiadiazol-4-yl-2,3,4- trifluorobenzenesulfonamide | 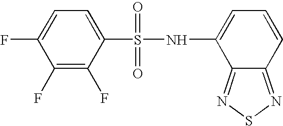 |
| 100 | N-2,1,3-benzothiadiazol-4-yl-2- cyanobenzenesulfonamide | 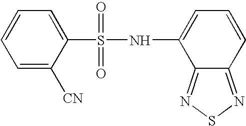 |
| 101 | N-[4-(4-nitrophenoxy)phenyl]-2,1,3- benzoxadiazole-4-sulfonamide | 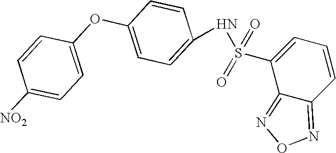 |
| 102 | N-(4-phenoxyphenyl)-2,1,3- benzoxadiazole-4-sulfonamide | 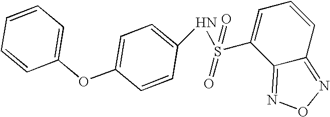 |
| 103 | N-(4-{[4-(trifluoromethyl)pyridin-2- yl]oxy}phenyl)-2,1,3-benzoxadiazole-4- sulfonamide | 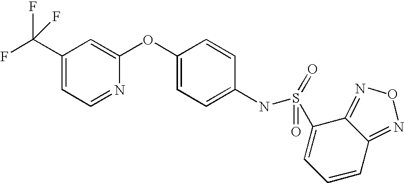 |
| 104 | N-[4-(4-chlorophenoxy)phenyl]-2,1,3- benzoxadiazole-4-sulfonamide | 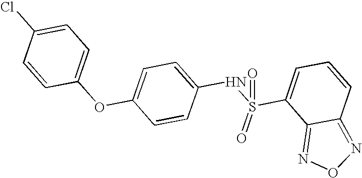 |
| 105 | {4-[4-chloro-3- (trifluoromethyl)phenyl]piperazin-1-yl}-7- nitro-2,1,3-benzoxadiazole | 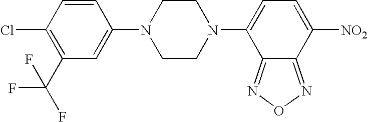 |
| 106 | 4-nitro-7-{4-[3- (trifluoromethyl)phenyl]piperazin-1-yl}- 2,1,3-benzoxadiazole | 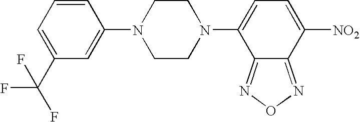 |
| 107 | 4-[4-(2-morpholin-4-yl-2- oxoethyl)piperazin-1-yl]-7-nitro-2,1,3- benzoxadiazole | 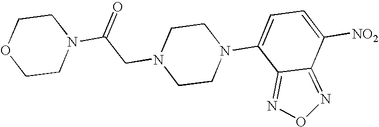 |
| 108 | 4-nitro-7-(4-pyridin-4-ylpiperazin-1-yl)- 2,1,3-benzoxadiazole | 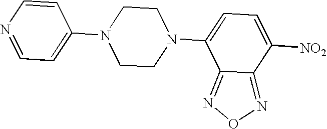 |
| 109 | 4-nitro-7-(1,3-(pyrimidin-2-yl)piperazin-1- yl)-2,1,3-benzoxadiazole | 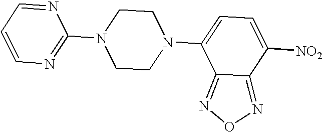 |
| 110 | 4-(4-methylpiperazin-1-yl)-7-nitro-2,1,3- benzoxadiazole | 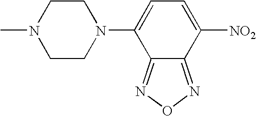 |
| 111 | N-methyl-N-[2-(methylamino)ethyl]-2,1,3- benzoxadiazole-4-sulfonamide | 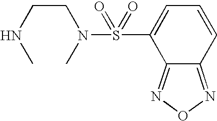 |
| 112 | 4-nitro-7-piperazin-1-yl-2,1,3-benzoxadiazole | 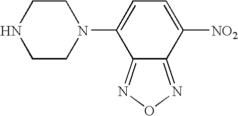 |
| 113 | N-2,1,3-benzothiadiazol-4-yl-3-chloro-4- [4-nitro-2- (trifluoromethyl)phenoxy]benzene- sulfonamide | 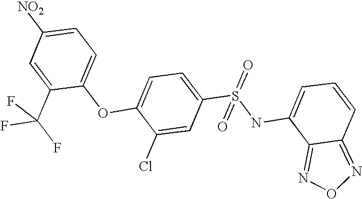 |
| 114 | N-2,1,3-benzoxadiazol-4-yl-3-chloro-4-[4- nitro-2- (trifluoromethyl)phenoxy]benzenesulfonamide | 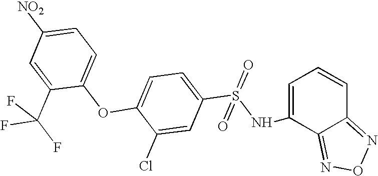 |
| 115 | N-2,1,3-benzothiadiazol-4-yl-5- phenoxypyridine-2-sulfonamide | 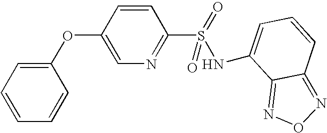 |
| 116 | N-2,1,3-benzothiadiazol-4-yl-4-(pyridin-3- yloxy)benzenesulfonamide | 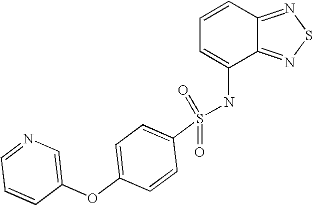 |
| 117 | N-2,1,3-benzothiadiazol-4-yl-6-[2-chloro- 4-(trifluoromethyl)phenoxy]pyridine-3- sulfonamide | 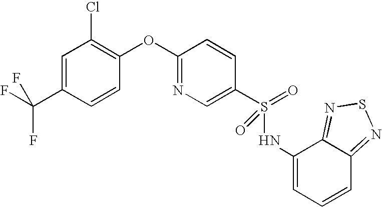 |
| 118 | 2-[4-(7-nitro-2,1,3-benzoxadiazol-4- yl)piperazin-1-yl]nicotinonitrile | 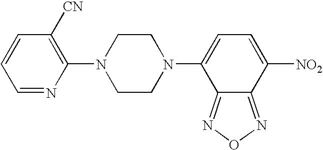 |
| 119 | 2-[4-(2,1,3-benzoxadiazol-4- ylsulfonyl)piperazin-1-yl]nicotinonitrile | 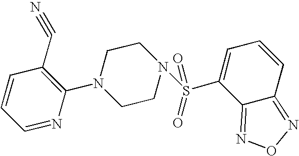 |
| 120 | 6-[4-(2,1,3-benzoxadiazol-4- ylsulfonyl)piperazin-1-yl]nicotinonitrile | 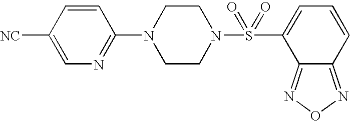 |
| 121 | 6-[4-(7-nitro-2,1,3-benzoxadiazol-4- yl)piperazin-1-yl]nicotinonitrile | 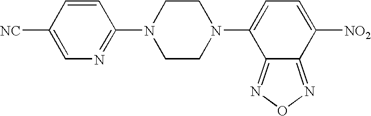 |
| 122 | 7-{4-[4-(trifluoromethyl)pyridin-2- yl]piperazin-1-yl}-2,1,3-benzoxadiazol-4- amide | 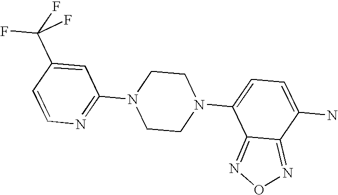 |
| 123 | 4-[(4-pyridin-2-ylpiperazin-1-yl)sulfonyl]- 2,1,3-benzoxadiazole | 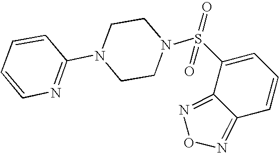 |
| 124 | 4-({4-[4-(trifluoromethyl)pyridin-2- yl]piperazin-1-yl}sulfonyl)-2,1,3- benzoxadiazole | 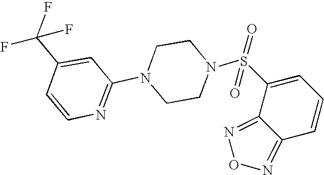 |
| 125 | 4({4-[3- (trifluoromethyl)phenyl]piperazin-1- yl}sulfonyl)-2,1,3-benzoxadiazole | 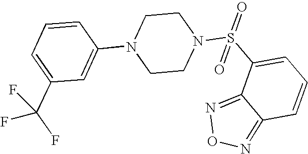 |
| 126 | 4-[(4-pyridin-4-ylpiperazin-1-yl)sulfonyl]- 2,1,3-benzoxadiazole | 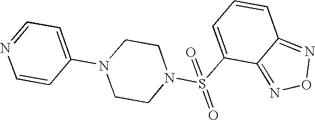 |
| 127 | 4-[4-(2-morpholin-4-ylethyl)piperazin-1- yl]sulfonyl}-2,1,3-benzoxadiazole | 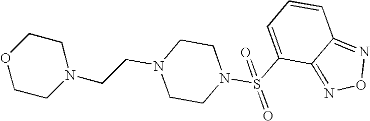 |
| 128 | 4-[(4-acetylpiperazin-1-yl)sulfonyl]-2,1,3- benzoxadiazole | 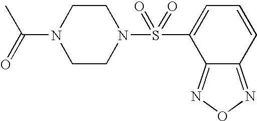 |
| 129 | 4-{[4-(2-morpholin-4-yl-2- oxoethyl)piperazin-1-yl]sulfonyl}-2,1,3- benzoxadiazole | 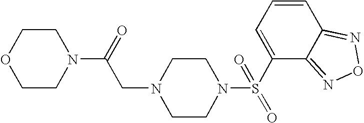 |
| 130 | 4-[(4-pyrimidin-2-ylpiperazin-1- yl)sulfonyl]-2,1,3-benzoxadiazole | 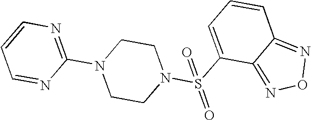 |
| 131 | 4-[(4-pyrazin-2-ylpiperazin-1-yl)sulfonyl]- 2,1,3-benzoxadiazole | 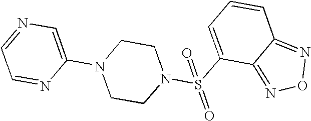 |
| 132 | 4-({4-[4-chloro-3- (trifluoromethyl)phenyl]piperazin-1- yl}sulfonyl)-2,1,3-benzoxadiazole | 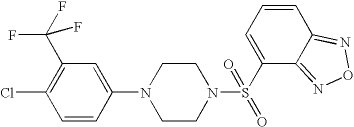 |
| 133 | 3-({7-[(5-methyl-1,3,4-thiadiazol-2- yl)thio]-4-nitro-2,1,3-benzoxadiazol-5- yl}amino)propyl acetate | 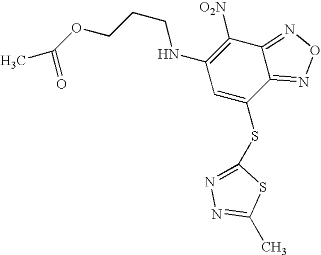 |
| 134 | 4-{[4-nitro-7-(propylthio)-2,1,3- benzoxadiazol-5-yl]amino}propyl acetate | 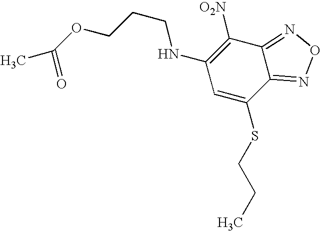 |
| 135 | 3-{[7-(cyclohexylthio)-4-nitro-2,1,3- benzoxadiazol-5-yl]amino}propyl acetate | 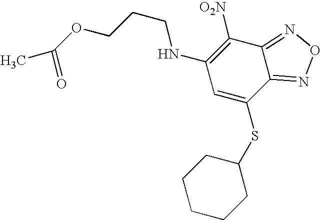 |
| 136 | 3-({7-[(2-furylmethyl)thio]-4-nitro-2,1,3- benzoxadiazol-5-yl}amino)propyl acetate | 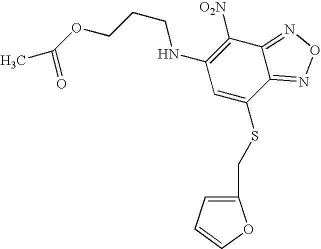 |
| 137 | ethyl [(6-{[3-(acetyloxy)propyl]amino}-7- nitro-2,1,3-benzoxadiazol-4-yl)thio]acetate | 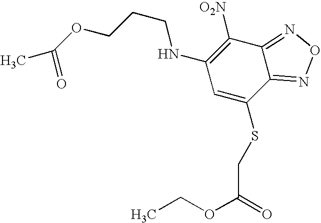 |
| 139 | 4-[4-(2,1,3-benzoxadiazol-4- ylsulfonyl)piperazin-1-yl]phenyl 2,1,3- benzoxadiazole-4-sulfonate | 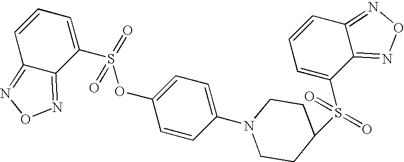 |
| 140 | 4-[4-(3,4-dimethylphenyl)piperazin-1-yl]- 7-nitro-2,1,3-benzoxadiazole | 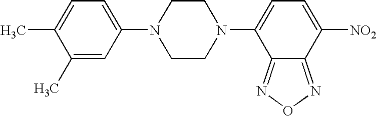 |
| 141 | 1-(7-nitro-2,1,3-benzoxadiazol-4-yl)-4- phenylpiperidine-4-carbonitrile | 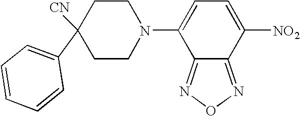 |
| 142 | (4-fluorophenyl)[1-(7-nitro-2,1,3- benzoxadiazol-4-yl)piperidin-4- yl]methanone | 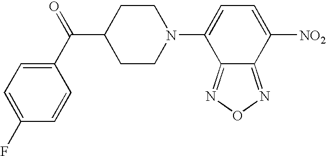 |
| 143 | 4-nitro-7-(4-phenylpiperazin-1-yl)-2,1,3- benzoxadiazole | 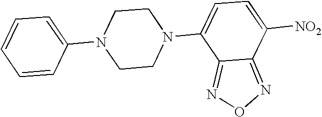 |
| 144 | 4-(3,5-dimethylpiperidin-1-yl)-7-nitro- 2,1,3-benzoxadiazole | 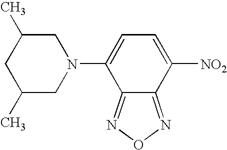 |
| 145 | 4-[4-(5-chloro-2-methylphenyl)piperazin- 1-yl]-7-nitro-2,1,3-benzoxadiazole | 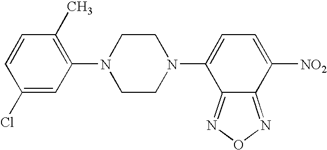 |
| 146 | 4-[4-(7-nitro-2,1,3-benzoxadiazol-4- yl)piperazin-1-yl]phenol | 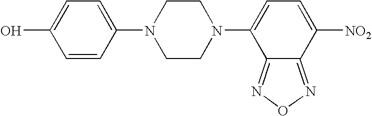 |
| 147 | 4-nitro-7-[4-(4-nitrophenyl)piperazin-1- yl]-2,1,3-benzoxadiazole | 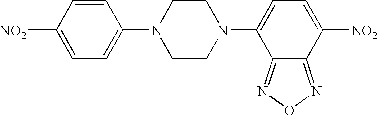 |
| 148 | 4-nitro-7-{4-[3-(trifluoromethyl)pyridin-2- yl]piperazin-1-yl}-2,1,3-benzoxadiazole | 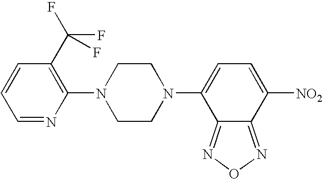 |
| 149 | 4-[(3-chlorophenyl)thio]-7-nitro-2,1,3- benzoxadiazole | 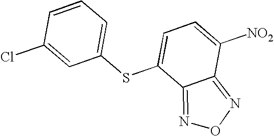 |
| 150 | 2-[(7-nitro-2,1,3-benzoxadiazol-4- yl)thio]ethanol | 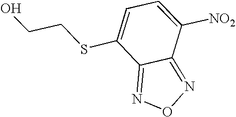 |
| 151 | 4-[4-(3,5-dichloropyridin-4-yl)piperazin-1- yl]-7-nitro-2,1,3-benzoxadiazole | 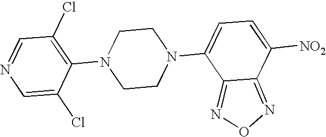 |
| 152 | 2-methyl-4-[4-(7-nitro-2,1,3- benzoxadiazol-4-yl)piperazin-1- yl]quinoline | 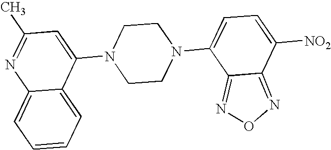 |
| 153 | ethyl 5-cyano-6-methyl-2-[4-(7-nitro- 2,1,3-benzoxadiazol-4-yl)piperazin-1- yl]nicotinate | 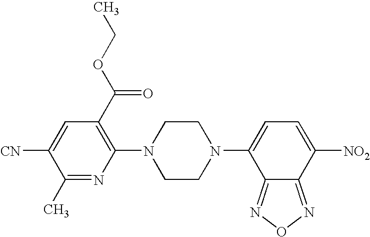 |
| 154 | 4-[4-(4-methylpyridin-2-yl)piperazin-1-yl]- 7-nitro-2,1,3-benzoxadiazole | 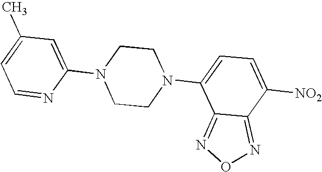 |
| 155 | 2-[4-(7-nitro-2,1,3-benzoxadiazol-4- yl)piperadin-1-yl]quinoline | 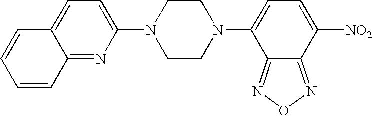 |
| 156 | 4-[4-(3-methylpyridin-2-yl)piperazin-1-yl]- 7-nitro-2,1,3-benzoxadiazole | 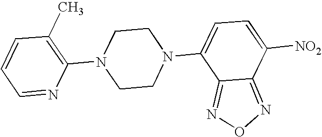 |
| 157 | 4-[(4-methylphenyl)thio]-7-nitro-2,1,3- benzoxadiazole | 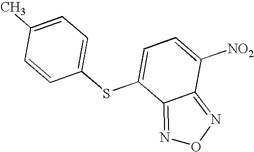 |
| 158 | 4-(1,3-benzothiazol-2-ylthio)-7-nitro-2,1,3- benzoxadiazole | 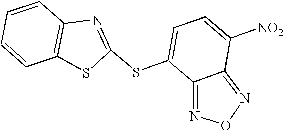 |
| 159 | 4-(4-chlorophenyl)-1-(7-nitro-2,1,3- benzoxadiazol-4-yl)piperidin-4-ol | 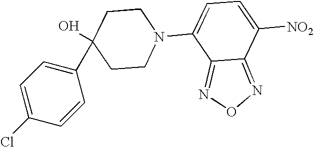 |
| 160 | 1-[1-(7-nitro-2,1,3-benzoxadiazol-4- yl)piperidin-4-yl]-1,3-dihydro-2H- benzimidazol-2-one | 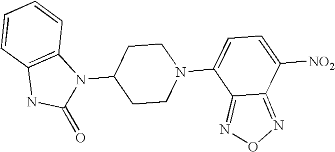 |
| 161 | 4-(2,6-dimethylmorpholin-4-yl)-7-nitro- 2,1,3-benzoxadiazole | 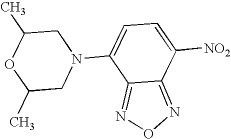 |
| 162 | 4-[4-(6-methylpyridin-2-yl)piperazin-1-yl]- 7-nitro-2,1,3-benzoxadiazole | 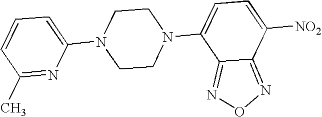 |
| 163 | 4-({4-[3-chloro-5-(trifluoromethyl)pyridin- 2-yl]piperazin-1-yl}sulfonyl)-7-methoxy- 2,1,3-benzoxadiazole | 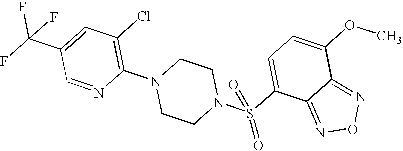 |
| 164 | 4-({4-[3-chloro-5-(trifluoromethyl)pyridin- 2-yl]piperazin-1-yl}sulfonyl)-7-fluoro- 2,1,3-benzoxadiazole | 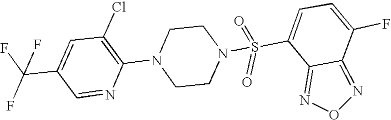 |
| 165 | 4-fluoro-7-({4-[3-(trifluoromethyl)pyridin- 2-yl]piperazin-1-yl}sulfonyl)-2,1,3- benzoxadiazole | 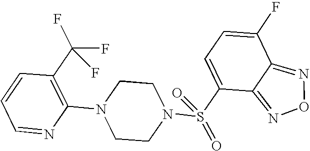 |
| 166 | 4-{4-[3-(trifluoromethyl)pyridin-2- yl]piperazin-1-yl}-7-({4-[3- (trifluoromethyl)pyridin-2-yl]piperazin-1- yl}sulfonyl)-2,1,3-benzoxadiazole | 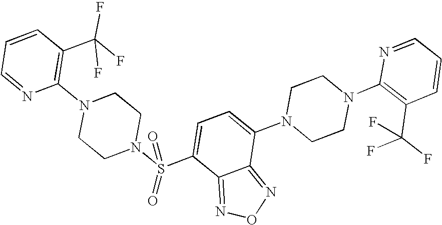 |
| 167 | methyl 4-{[(7-fluoro-2,1,3-benzoxadiazol- 4-yl)sulfonyl]amino}benzoate | 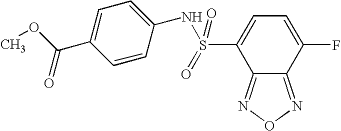 |
| 168 | 1-[(7-chloro-2,1,3-benzoxadiazol-4- yl)sulfonyl]-4-(4-chlorophenyl)piperidin-4-ol | 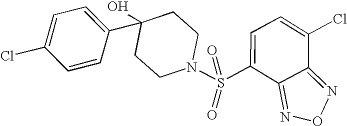 |
| 169 | 4-chloro-7-({4-[3-chloro-5- (trifluoromethyl)pyridin-2-yl]piperazin-1- yl}sulfonyl)-2,1,3-benzoxadiazole | 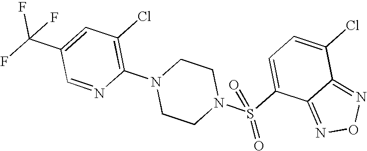 |
| 170 | 2-{4-[(7-chloro-2,1,3-benzoxadiazol-4- yl)sulfonyl]piperazin-1-yl}nicotinomtrile | 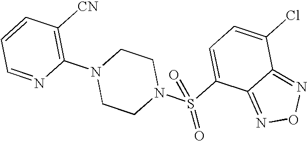 |
| 171 | 4-{4-[(7-chloro-2,1,3-benzoxadiazol-4- yl)sulfonyl]piperazin-1-yl}phenyl 7- chloro-2,1,3-benzoxadiazole-4-sulfonate | 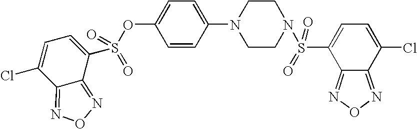 |
| 172 | 4-{4-[(7-chloro-2,1,3-benzoxadiazol-4- yl)sulfonyl]piperazin-1-yl}phenol | 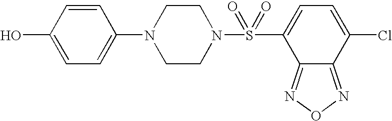 |
| 173 | 4-[4-({7-[4-(4-hydroxyphenyl)piperazin-1- yl]-2,1,3-benzoxadiazol-4- yl}sulfonyl)piperazin-1-yl]phenol | 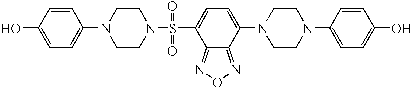 |
| 174 | methyl 4-({[7-(quinolin-8-ylthio)-2,1,3- benzoxadiazol-4- yl]sulfonyl}amino)benzoate | 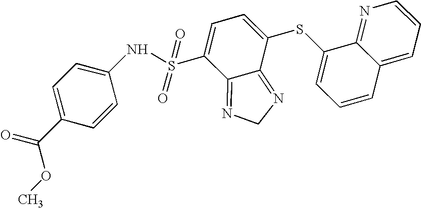 |
| 175 | 4-(4-chlorophenyl)-1-{[7-(quinolin-8- ylthio)-2,1,3-benzoxadiazol-4- yl]sulfonyl}piperidin-4-ol | 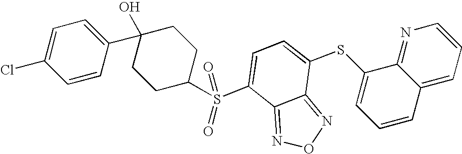 |
| 176 | 2-(4-{[7-(quinolin-8-ylthio)-2,1,3- benzoxadiazol-4-yl]sulfonyl}piperazin-1- yl)nicotinonitrile | 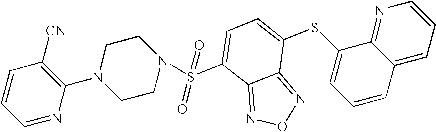 |
| 177 | 8-{[7-({4-[3-chloro-5- (trifluoromethyl)pyridin-2-yl]piperazin-1- yl}sulfonyl)-2,1,3-benzoxadiazol-4- yl]thio}quinoline | 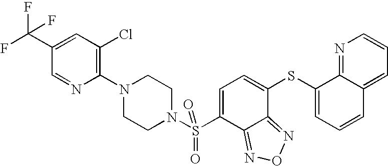 |
| 178 | methyl 4-{[(5-chloro-2,1,3-benzoxadiazol- 4-yl)sulfonyl]amino}benzoate | 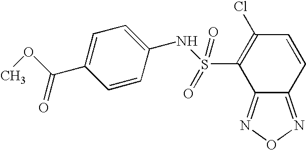 |
| 179 | 5-chloro-4-({4-[3-chloro-5- (trifluoromethyl)pyridin-2-yl]piperazin-1- yl}sulfonyl)-2,1,3-benzoxadiazole | 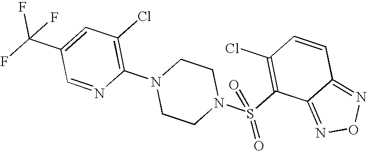 |
| 180 | 2-{4-[(5-chloro-2,1,3-benzoxadiazol-4- yl)sulfonyl]piperazin-1-yl}benzonitrile | 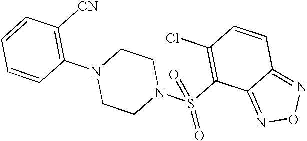 |
| 181 | 5-{4-[4-chloro-3- (trifluoromethyl)phenyl]piperazin-1-yl}-4- ({4-[4-chloro-3- (trifluoromethyl)phenyl]piperazin-1- yl}sulfonyl)-2,1,3-benzoxadiazole | 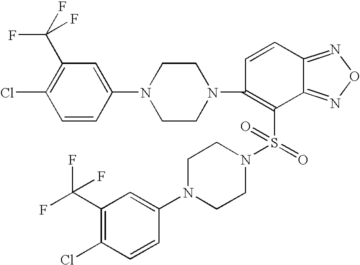 |
| 182 | 5-chloro-4-({4-[4-chloro-3- (trifluoromethyl)phenyl]piperazin-1- yl}sulfonyl)-2,1,3-benzoxadiazole | 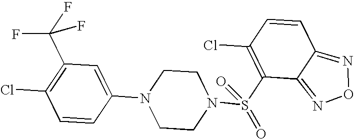 |
| 183 | 2-(4-{[5-(quinolin-8-ylthio)-2,1,3- benzoxadiazol-4-yl]sulfonyl}piperazin-1- yl)nicotinonitrile | 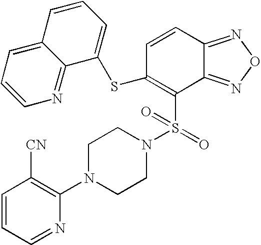 |
| 184 | 4-chloro-7-[(4-pyrimidin-2-ylpiperazin-1- yl)sulfonyl]-2,1,3-benzoxadiazole | 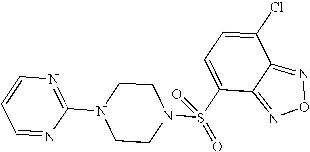 |
| 185 | 2-(2-{4-[(7-chloro-2,1,3-benzoxadiazol-4- yl)sulfonyl]piperazin-1-yl}ethoxy)ethanol | 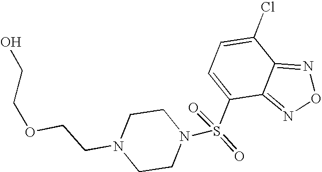 |
| 186 | 4-chloro-7-[(4-phenylpiperazin-1- yl)sulfonyl]-2,1,3-benzoxadiazole | 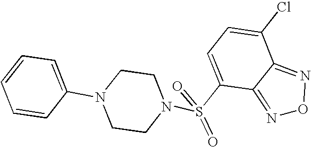 |
| 187 | 4-(4-phenylpiperazin-1-yl)-7-[(4- phenylpiperazin-1-yl)sulfonyl]-2,1,3- benzoxadiazole | 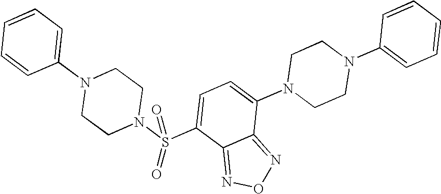 |
| 188 | 1-{1-[(7-chloro-2,1,3-benzoxadiazol-4- yl)sulfonyl]piperidin-4-yl}-1,3-dihydro- 2H-benzimidazol-2-one | 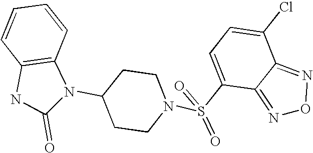 |
| 189 | 4-chloro-7-[(4-methylpiperazin-1- yl)sulfonyl]-2,1,3-benzoxadiazole | 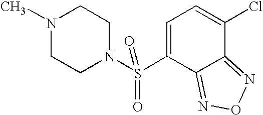 |
| 190 | 1-(1-{[7-(quinolin-8-ylthio)-2,1,3- benzoxadiazol-4-yl]sulfonyl}piperidin-4- yl)-1,3-dihydro-2H-benzimidazol-2-one | 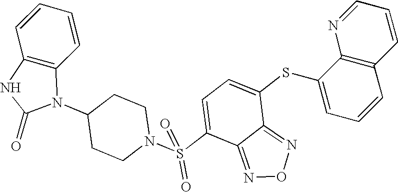 |
| 191 | 8-({7-[(4-methylpiperazin-1-yl)sulfonyl]- 2,1,3-benzoxadiazol-4-yl}thio)quinoline | 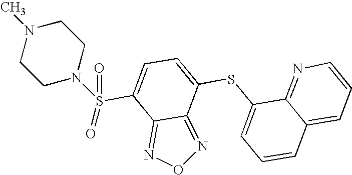 |
| 192 | 4-chloro-7-[(3,5-dimethylpiperidin-1- yl)sulfonyl]-2,1,3-benzoxadiazole | 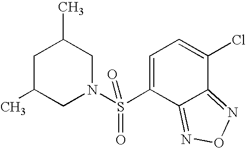 |
| 193 | 4-(3,5-dimethylpiperidin-1-yl)-7-[(3,5- dimethylpiperidin-1-yl)sulfonyl]-2,1,3- benzoxadiazole | 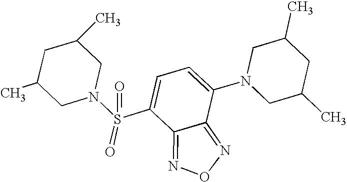 |
| 194 | 4-chloro-7-{[4-(6-methylpyridin-2- yl)piperazin-1-yl]sulfonyl}-2,1,3- benzoxadiazole | 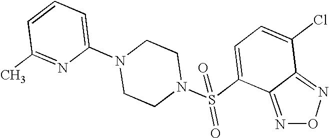 |
| 195 | 7-amino-N-(2-chloropyridin-4-yl)-2,1,3- benzoxadiazole-4-sulfonamide | 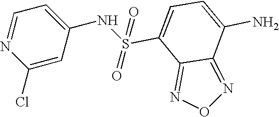 |
| 196 | 8-[(7-{[4-(6-methylpyndin-2-yl)piperazin- 1-yl]sulfonyl}-2,1,3-benzoxadiazol-4- yl)thio]quinoline | 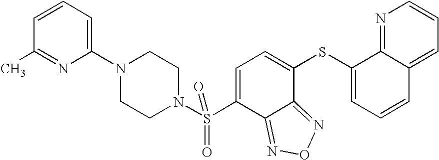 |
| 197 | 8-({7-[(3,5-dimethylpipendin-1- yl)sulfonyl]-2,1,3-benzoxadiazol-4- yl}thio)quinoline | 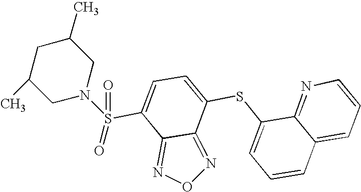 |
| 198 | 7-chloro-N-(6-ethoxy-1,3-benzothiazol-2- yl)-2,1,3-benzoxadiazole-4-sulfonamide | 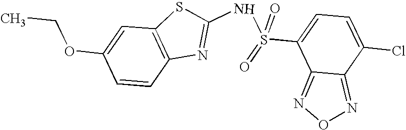 |
| 199 | 1-[(7-hydroxy-2,1,3-benzoxadiazol-4- yl)sulfonyl]-4-phenylpiperidine-4- carbonitrile | 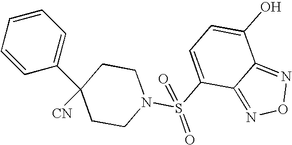 |
| 200 | 5-chloro-4-[(4-pyrazin-2-ylpiperazin-1- yl)sulfonyl]-2,1,3-benzoxadiazole | 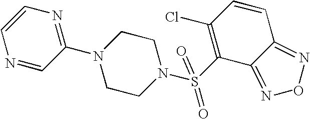 |
| 201 | 4-[(4-acetylpiperazin-1-yl)sulfonyl]-5- chloro-2,1,3-benzoxadiazole | 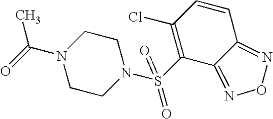 |
| 202 | 5-chloro-4-{[4-(6-methylpyridin-2- yl)piperazin-1-yl]sulfonyl}-2,1,3- benzoxadiazole | 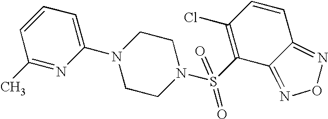 |
| 203 | 5-[4-(6-methylpyridin-2-yl)piperazin-1-yl]- 4-{[4-(5-methylpyridin-2-yl)piperazin-1- yl]sulfonyl}-2,1,3-benzoxadiazole | 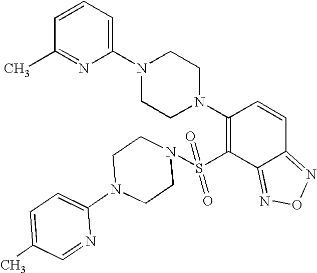 |
| 204 | 5-chloro-4-[(2,6-dimethylmorpholin-4- yl)sulfonyl]-2,1,3-benzoxadiazole | 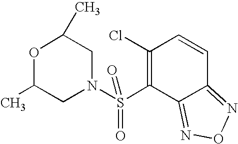 |
| 205 | 5-(2,6-dimethylmorpholin-4-yl)-4-[(2,6- dimethylmorpholin-4-yl)sulfonyl]-2,1,3- benzoxadiazole | 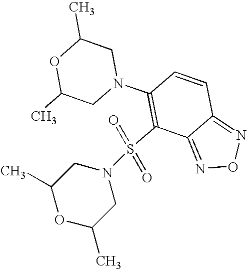 |
| 206 | 8-[(4-{[4-(6-methylpyridin-2-yl)piperazin- 1-yl]sulfonyl}-2,1,3-benzoxadiazol-5- yl)thio]quinoline | 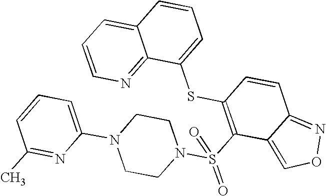 |
| 207 | 8-({4-[(4-pyrazin-2-ylpiperazin-1- yl)sulfonyl]-2,1,3-benzoxadiazol-5- yl}thio)quinoline | 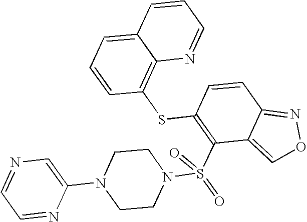 |
| 209 | 6-{4-[(5-chloro-2,1,3-benzoxadiazol-4- yl)sulfonyl]piperazin-1-yl}nicotinonitrile | 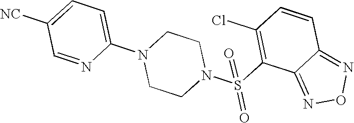 |
| 210 | 6-[4-({5-[4-(5-cyanopyridin-2- yl)piperazin-1-yl]-2,1,3-benzoxadiazol-4- yl}sulfonyl)piperazin-1-yl]nicotinonitrile | 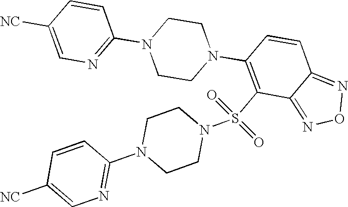 |
| 211 | 4-(2,6-dimethylmorpholin-4-yl)-7-[(2,6- dimethylmorpholin-4-yl)sulfonyl]-2,1,3- benzoxadiazole | 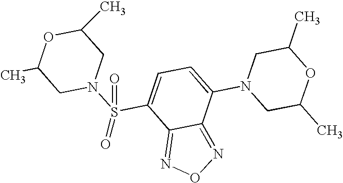 |
| 212 | 6-(4-{[5-(quinolin-8-ylthio)-2,1,3- benzoxadiazol-4-yl]sulfonyl}piperazin-1- yl)nicotinonitrile | 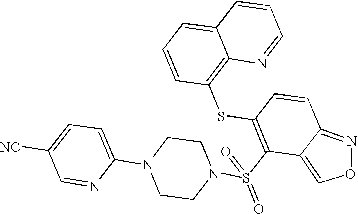 |
| 213 | 8-({7-[(2,6-dimethylmorpholin-4- yl)sulfonyl]-2,1,3-benzoxadiazol-4- yl}thio)quinoline | 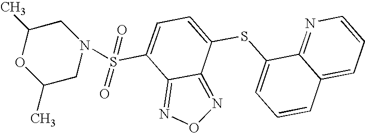 |
| 214 | 8-({4-[(4-acetylpiperazin-1-yl)sulfonyl]- 2,1,3-benzoxadiazol-5-yl}thio)quinoline | 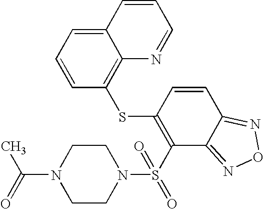 |
| 215 | methyl 4-({[5-(quinolin-8-ylthio)-2,1,3- benzoxadiazol-4- yl]sulfonyl}amino)benzoate | 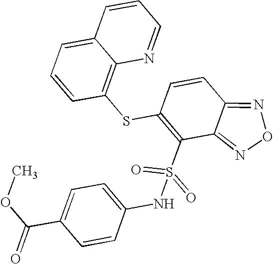 |
| 216 | 8-({4-[(2,6-dimethylmorpholin-4- yl)sulfonyl]-2,1,3-benzoxadiazol-5- yl}thio)quinoline | 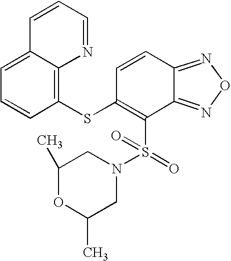 |
| 217 | 8-({7-[(4-phenylpiperazin-1-yl)sulfonyl]- 2,1,3-benzoxadiazol-4-yl}thio)quinoline | 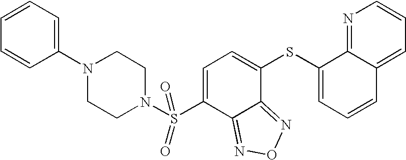 |
| 218 | 7-chloro-N-1H-indazol-5-yl-2,1,3- benzoxadiazole-4-sulfonamide | 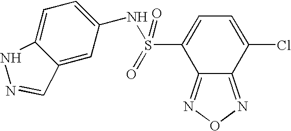 |
| 219 | 5-chloro-N-1H-indazol-6-yl-2,1,3- benzoxadiazole-4-sulfonamide | 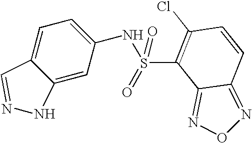 |
| 220 | 7-chloro-N-1 H-indazol-6-yl-2,1,3- benzoxadiazole-4-sulfonamide | 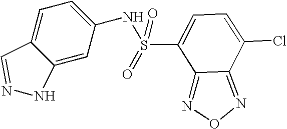 |
| 221 | 1-[(7-chloro-2,1,3-benzoxadiazol-4- yl)sulfonyl]-1H-indazol-6-amine | 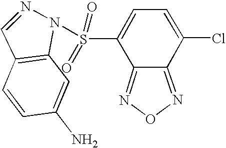 |
| 222 | N-1H-indazol-5-yl-7-nitro-2,1,3- benzoxadiazol-4-amine | 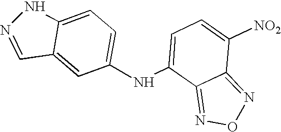 |
| 223 | N-1H-indazol-6-yl-7-nitro-2,1,3- benzoxadiazol-4-amine | 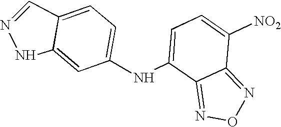 |
| 224 | N-1H-indazol-5-yl-7-(quinolin-8-ylthio)- 2,1,3-benzoxadiazole-4-sulfonamide | 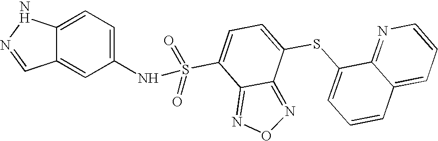 |
| 225 | 4-chloro-7-{[4-(pyrimidin-2- yloxy)pipendin-1-yl]sulfonyl}-2,1,3- benzoxadiazole | 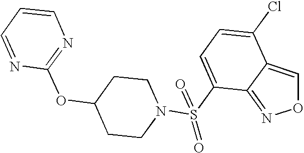 |
| 226 | 4-[(7-{[4-(pyrimidin-2-yloxy)piperidin-1- yl]sulfonyl}-2,1,3-benzaxadiazol-4- yl)thio]phenol | 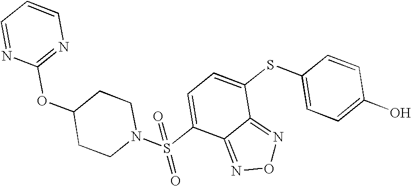 |
| 227 | 1-{1-[(7-chloro-2,1,3-benzoxadiazol-4- yl)sulfonyl]-4-phenylpiperidin-4- yl}ethanone | 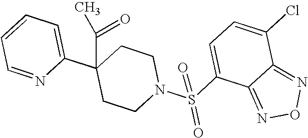 |
| 228 | 4-(4-chlorophenyl)-1-({7-[(4- hydroxyphenyl)thio]-2,1,3-benzoxadiazol- 4-yl}sulfonyl)piperidin-4-ol | 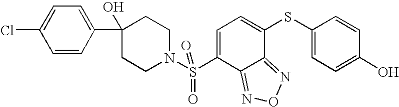 |
| 229 | 5-chloro-4-({4-[2- (trifluoromethyl)phenoxy]piperidin-1- yl}sulfonyl)-2,1,3-benzoxadiazole | 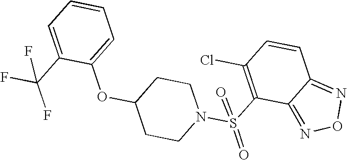 |
| 230 | 4-chloro-7-({4-[2- (trifluoromethyl)phenoxy]piperidin-1- yl}sulfonyl)-2,1,3-benzoxadiazole | 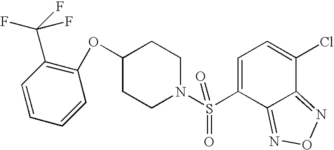 |
| 231 | 1-[1-(7-nitro-2,1,3-benzoxadiazol-4-yl)-4- phenylpiperidin-4-yl]ethanone | 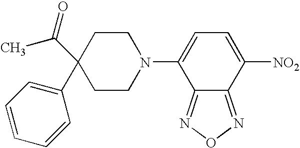 |
| 232 | 4-fluoro-7-({4-[2- (trifluoromethyl)phenoxy]piperidin-1- yl}sulfonyl)-2,1,3-benzoxadiazole | 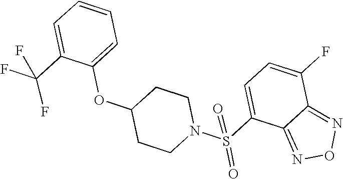 |
| 233 | 4-nitro-7-{4-[2- (trifluoromethyl)phenoxy]piperidin-1-yl}- 2,1,3-benzoxadiazole | 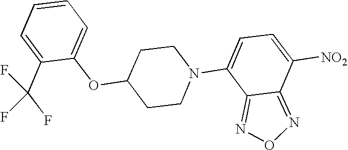 |
| 234 | 5-chloro-4-[[(1,3-pyrimidin-2- yloxy)piperidin-1-yl]sulfonyl]piperazin-1- yl]-2,1,3-benzoxadiazole | 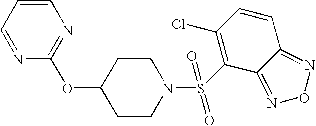 |
| 235 | 4-[4-(1,3-pyrimidin-2-yloxy)piperazin-1- ylsulsonyl]-2,1,3-benzoxadiazole | 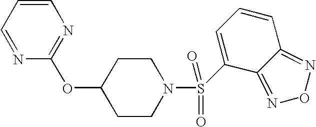 |
| 236 | 5-[4-(1,3-pyrimidin-2-yloxy)piperidin-1- yl]-4-{[4-(1,3-pyrimidin-2- yloxy)piperidin-1-yl]sulfonyl}-2,1,3- benzoxadiazole | 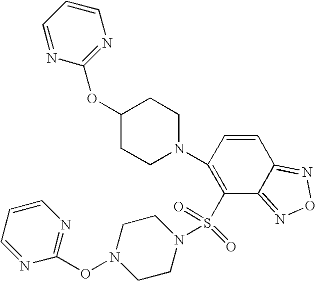 |
| 237 | 4-nitro-7-thiomorpholin-4-yl-2,1,3- benzoxadiazole | 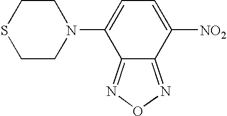 |
| 238 | 2-[(7-nitro-2,1,3-benzoxadiazol-4- yl)thio]quinoline | 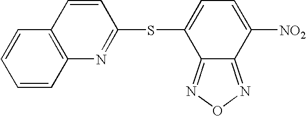 |
| 240 | 4-({4-[2- (trifluoromethyl)phenoxy]piperidin-1- yl}sulfonyl)-2,1,3-benzothiadiazole | 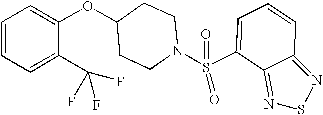 |
| 242 | 4-(4,4-dimethylpipendin-1-yl)-7-nitro- 2,1,3-benzoxadiazole | 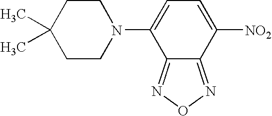 |
| 243 | 4-[(4,4-dimethylpiperidin-1-yl)sulfonyl]-7- fluoro-2,1,3-benzoxadiazole | 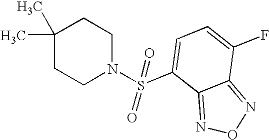 |
| 245 | 5-chloro-4-[(4,4-dimethylpiperidin-1- yl)sulfonyl]-2,1,3-benzoxadiazole | 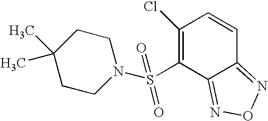 |
| 246 | 4-(4-chlorophenyl)-1-{[7-(pyridin-4- ylthio)-2,1,3-benzoxadiazol-4- yl]sulfonyl}piperidin-4-ol | 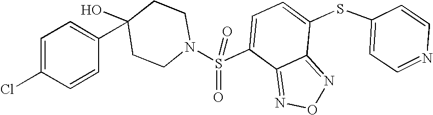 |
| 247 | 4-chloro-7-[(4,4-dimethylpiperidin-1- yl)sulfonyl]-2,1,3-benzoxadiazole | 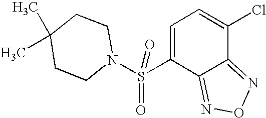 |
| 248 | methyl 4-({[7-(quinolin-2-ylthio)-2,1,3- benzoxadiazol-4- yl]sulfonyl}amino)benzoate | 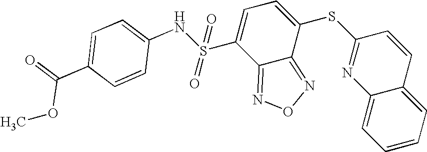 |
| 249 | 4-({7-[(4-phenylpiperazin-1-yl)sulfonyl]- 2,1,3-benzoxadiazol-4-yl}thio)phenol | 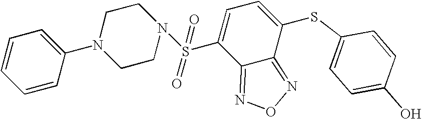 |
| 250 | methyl 4-{[(7-thiomorpholin-4-yl-2,1,3- benzoxadiazol-4- yl)sulfonyl]amino}benzoate | 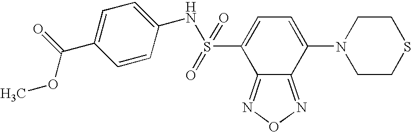 |
| 251 | 4-[(4-phenylpiperazin-1-yl)sulfonyl]-7- (pyridin-4-ylthio)-2,1,3-benzoxadiazole | 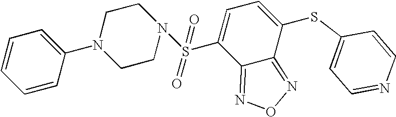 |
| 252 | 2-({7-[(4-phenylpiperazin-1-yl)sulfonyl]- 2,1,3-benzoxadiazol-4-yl}thio)quinoline | 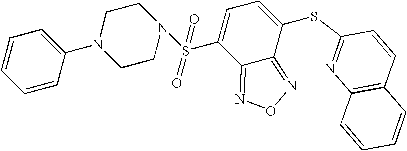 |
| 253 | 4-(1-naphthylthio)-7-[(4-phenylpiperazin- 1-yl)sulfonyl]-2,1,3-benzoxadiazole | 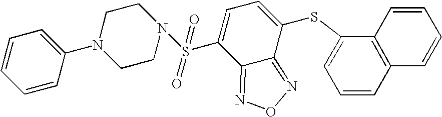 |
| 254 | 4-[(2-methoxyphenyl)thio]-7-[(4- phenylpiperazin-1-yl)sulfonyl]-2,1,3- benzoxadiazole | 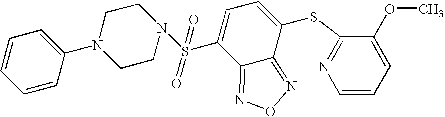 |
| 255 | 8-({7-[(4-phenylpiperazin-1-yl)sulfonyl]- 2,1,3-benzoxadiazol-4-yl}oxy)quinoline | 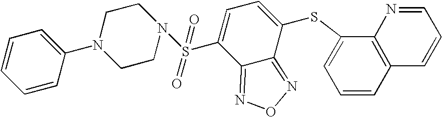 |
| 256 | methyl 4-({[7-(pyridin-4-ylthio)-2,1,3- benzoxadiazol-4- yl]sulfonyl}amino)benzoate | 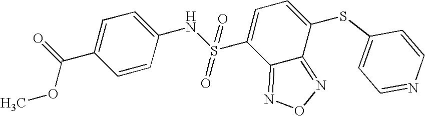 |
| 257 | 1′-(2,1,3-benzothiadiazol-4-ylsulfonyl)- 3H-spiro[2-benzofiiran-1,4′-piperidine] | 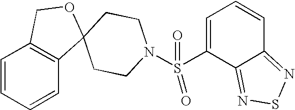 |
| 258 | 1′-[(5-chloro-2,1,3-benzoxadiazol-4- yl)sulfonyl]-3H-spiro[2-benzofuran-1,4′- piperidine] | 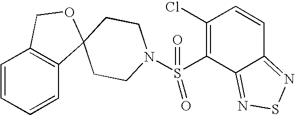 |
| 259 | 1′-[(7-fluoro-2,1,3-benzoxadiazol-4- yl)sulfonyl]-3H-spiro[2-benzofuran-1,4′- piperidine] | 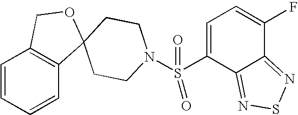 |
| 260 | 1′-[(7-chloro-2,1,3-benzoxadiazol-4- yl)sulfonyl]-3H-spiro[2-benzofuran-1,4′- piperidine] | 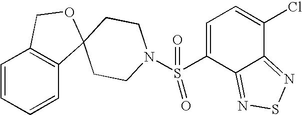 |
| 261 | 4-[(4-methylpiperazin-1-yl)sulfonyl]-7-(2- naphthylthio)-2,1,3-benzoxadiazole | 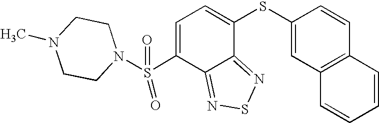 |
| 262 | 7-chloro-N-(3,4-difluorophenyl)-2,1,3- benzoxadiazole-4-sulfonamide | 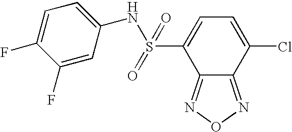 |
| 263 | 7-chloro-N-(3-fluorophenyl)-2,1,3- benzoxadiazole-4-sulfonamide | 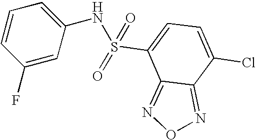 |
| 264 | N-(3,4-difluorophenyl)-7-[(3,4- difluorophenyl)amino]-2,1,3- benzoxadiazole-4-sulfonamide | 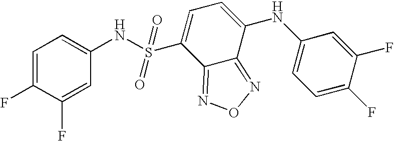 |
| 265 | 8-({7-[(4-methylpiperazin-1-yl)sulfonyl]- 2,1,3-benzoxadiazol-4-yl}thio)quinoline | 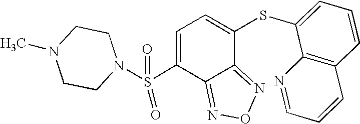 |
| 266 | 4-({7-[(4-methylpiperazin-1-yl)sulfonyl]- 2,1,3-benzoxadiazol-4-yl}thio)phenol | 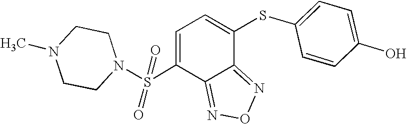 |
| 267 | 4-[(4-methylpiperazin-1-yl)sulfonyl]-7-(1- naphthylthio)-2,1,3-benzoxadiazole | 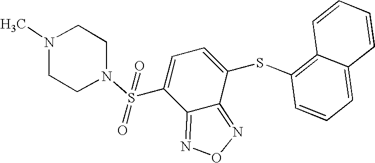 |
| 268 | 2-({7-[(4-methylpiperazin-1-yl)sulfonyl]- 2,1,3-benzoxadiazol-4-yl}thio)quinoline | 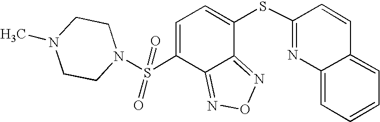 |
| 269 | 7-chloro-N-(5-chloro-2-methoxyphenyl)- 2,1,3-benzoxadiazole-4-sulfonamide | 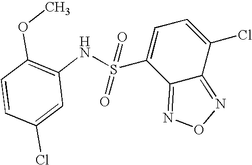 |
| 270 | 4-[(2-methoxyphenyl)thio]-7-[(4- methylpiperazin-1-yl)sulfonyl]-2,1,3- benzoxadiazole | 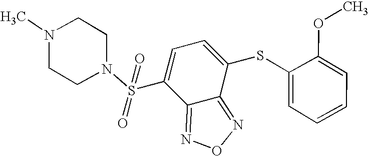 |
| 271 | 7-chloro-N-(2,3-difluorophenyl)-2,1,3- benzoxadiazole-4-sulfonamide | 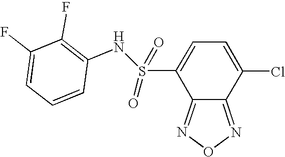 |
| 272 | 7-fluoro-N-(3-fluorophenyl)-2,1,3- benzoxadiazole-4-sulfonamide | 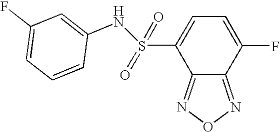 |
| 273 | N-(3-fluorophenyl)-7-[(3- fluorophenyl)amino]-2,1,3- benzoxadiazole-4-sulfonamide | 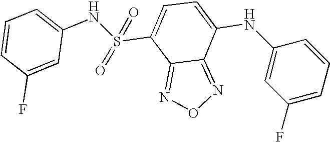 |
| 274 | N-(2,3-difluorophenyl)-7-fluoro-2,1,3- benzoxadiazole-4-sulfonamide | 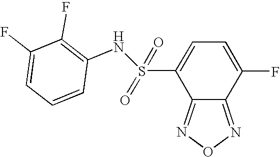 |
| 275 | N-(4-fluoro-2-methylphenyl)-7-[(4-fluoro- 2-methylphenyl)amino]-2,1,3- benzoxadiazole-4-sulfonamide | 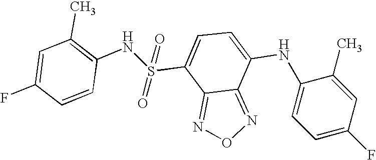 |
| 276 | 7-fluoro-N-(4-fluoro-2-methylphenyl)- 2,1,3-benzoxadiazole-4-sulfonamide | 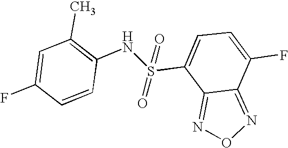 |
| 277 | methyl 4-({[7-(quinolin-8-yloxy)-2,1,3- benzoxadiazol-4- yl]sulfonyl}amino)benzoate | 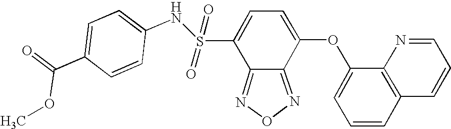 |
| 278 | benzyl 4-(7-nitro-2,1,3-benzoxadiazol-4- yl)piperazine-1-carboxylate | 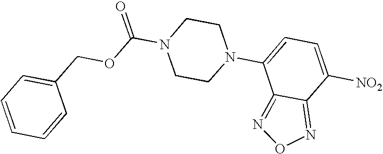 |
| 279 | 7-{4-[3-chloro-5-(trifluoromethyl)pyridin- 2-yl]piperazin-1-yl}-2,1,3-benzoxadiazol- 4-amine | 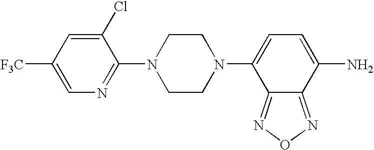 |
| 280 | 4-nitro-7-[4-(pyrimidin-2-yloxy)piperidin- 1-yl]-2,1,3-benzoxadiazole | 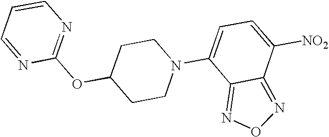 |
| 281 | N-(7-{4-[3-chloro-5- (trifluoromethyl)pyridin-2-yl]piperazin-1- yl}-2,1,3-benzoxadiazol-4-yl)thiophene-2- carboxamide | 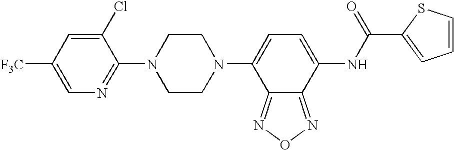 |
| 282 | 4-{4-[3-chloro-5-(trifluoromethyl)pyridin- 2-yl]piperazin-1-yl}-7-morpholin-4-yl- 2,1,3-benzoxadiazole | 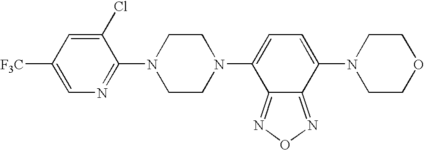 |
| 283 | 4-nitro-7-(4-pyridin-4-ylpiperidin-1-yl)- 2,1,3-benzoxadiazole | 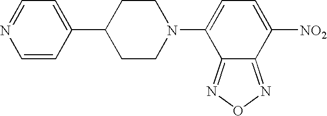 |
| 284 | 1′-(7-nitro-2,1,3-benzoxadiazol-4-yl)-3H- spiro[2-benzofuran-1,4′-piperidine] | 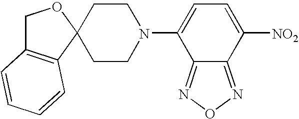 |
| 285 | 4-(3,5-dimethylphenyl)-2,1,3- benzoxadiazole | 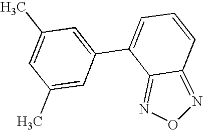 |
| 286 | 4-(3,5-dimethylphenyl)-7-nitro-2,1,3- benzoxadiazole | 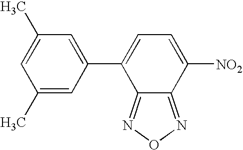 |
| 287 | 4-nitro-7-[4-(pyridin-2-yloxy)piperidin-1- yl]-2,1,3-benzoxadiazole | 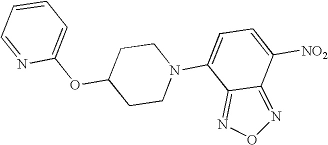 |
| 288 | 4-nitro-7-[4-(pyridin-2-ylthio)piperidin-1- yl]-2,1,3-benzoxadiazole | 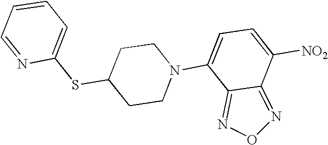 |
| 289 | 4-nitro-7-[4-(pyridin-2- ylsulfonyl)piperidin-1-yl]-2,1,3- benzoxadiazole | 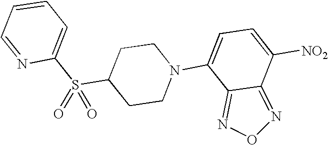 |
| 290 | 8-[(7-nitro-2,1,3-benzoxadiazol-4- yl)sulfonyl]quinoline | 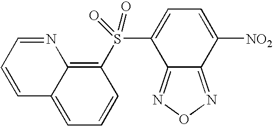 |
| 291 | 8-[(7-nitro-2,1,3-benzoxadiazol-4- yl)sulfinyl]quinoline | 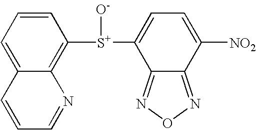 |
| 292 | 7-{4-[4-chloro-3- (trifluoromethyl)phenyl]piperazin-1-yl}- N,N-diethyl-4-nitro-2,1,3-benzoxadiazol- 5-amine | 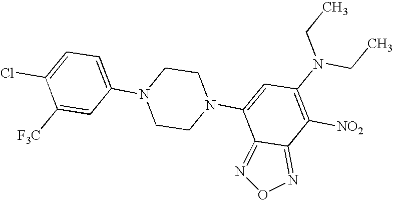 |
| 293 | 7-{4-[3-chloro-5-(trifluoromethyl)pyridin- 2-yl]piperazin-1-yl}-N,N-diethyl-4-nitro- 2,1,3-benzoxadiazol-5-amine | 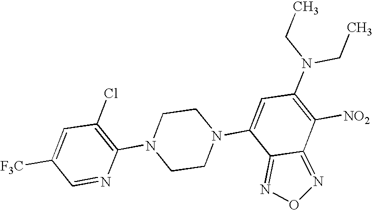 |
| 294 | 2-{4-[6-(diethylamino)-7-nitro-2,1,3- benzoxadiazol-4-yl]piperazin-1- yl}nicotinonitrile | 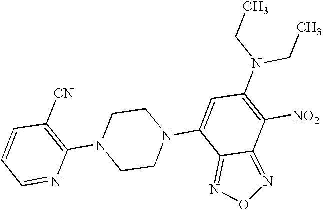 |
| 295 | 7-{4-[4-chloro-3- (trifluoromethyl)phenyl]piperazin-1-yl}-4- nitro-5-piperidin-1-yl-2,1,3- benzoxadiazole | 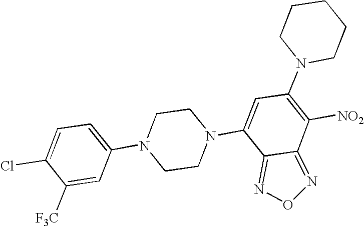 |
| 296 | 7-{4-[3-chloro-5-(trifluoromethyl)pyridin- 2-yl]piperazin-1-yl}-4-nitro-5-piperidin-1- yl-2,1,3-benoxadiazole | 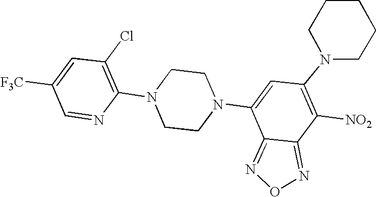 |
| 297 | 2-[4-(7-nitro-6-piperidin-1-yl-2,1,3- benzoxadiazol-4-yl)piperazin-1- yl]nicotinonitrile | 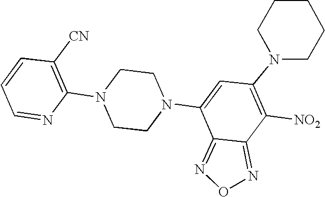 |
| 298 | N,N-diethyl-4-nitro-7-(1′H,3H-spiro[2- benzofuran-1,4′-piperidin]-1′-yl)-2,1,3- benzoxadiazol-5-amine | 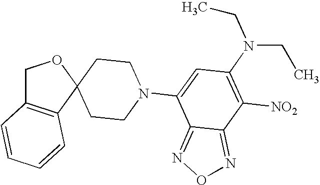 |
| 299 | 1-[6-(diethylamino)-7-nitro-2,1,3- benzoxadiazol-4-yl]-4-phenylpiperidin-4-ol | 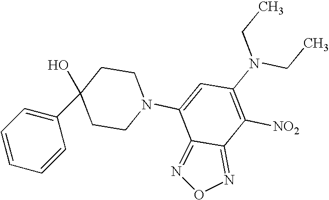 |
| 300 | 1′-(7-nitro-6-piperidin-1-yl-2,1,3- benzoxadiazol-4-yl)-3H-spiro[2- benzofuran-1,4′-piperidine] 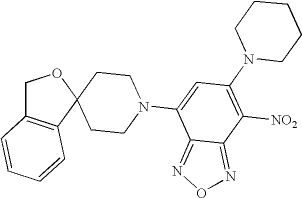 | |
| 301 | 1-(7-nitro-6-piperidin-1-yl-2,1,3- benzoxadiazol-4-yl)-4-phenylpiperidin-4-ol | 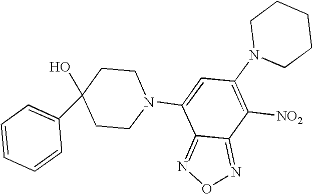 |
| 302 | 1′-(7-nitro-2,1,3-benzoxadiazol-4-yl)-3H- spiro[2-benzofuran-1,4′-piperidin]-3-one | 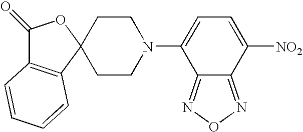 |
| 303 | 1′-[6-(diethylamino)-7-nitro-2,1,3- benzoxadiazol-4-yl]-3H-spiro[2- benzofuran-1,4′-piperidin]-3-one | 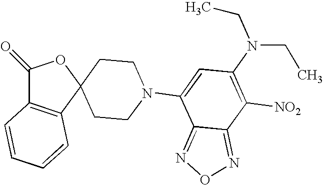 |
| 304 | 1′-(7-nitro-6-piperidin-1-yl-2,1,3- benzoxadiazol-4-yl)-3H-spiro[2- benzofuran-1,4′-piperidin]-3-one | 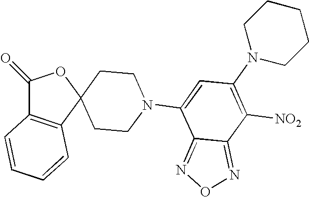 |
| 305 | 4-[3-(trifluoromethyl)phenyl]-2,1,3- benzoxadiazole | 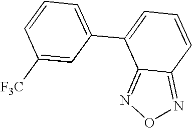 |
| 306 | 4-(3-methylphenyl)-2,1,3-benzoxadiazole | 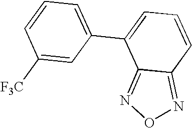 |
| 307 | 4-(4-tert-butylphenyl)-2,1,3-benzoxadiazole | 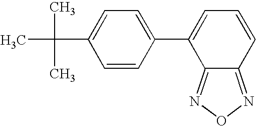 |
| 308 | 4-(3,5-difluorophenyl)-7-nitro-2,1,3- benzoxadiazole | 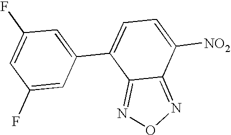 |
| 309 | 4-nitro-7-[3-(trifluoromethyl)phenyl]- 2,1,3-benzoxadiazole | 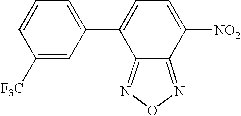 |
| 310 | 4-(3-methylphenyl)-7-nitro-2,1,3- benzoxadiazole | 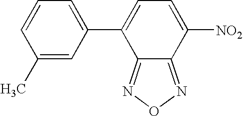 |
| 311 | 4-nitro-7-[4-(trifluoromethyl)phenyl]- 2,1,3-benzoxadiazole | 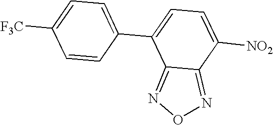 |
| 312 | 4-[2,4-bis(trifluoromethyl)phenyl]-7-nitro- 2,1,3-benzoxadiazole | 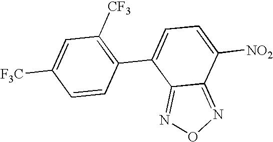 |
| 313 | 4-(4-tert-butylphenyl)-7-nitro-2,1,3- benzoxadiazole | 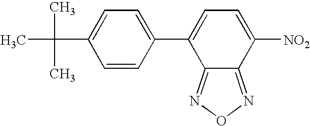 |
| 314 | 4-(2-fluoro-3-methoxyphenyl)-7-nitro- 2,1,3-benzoxadiazole | 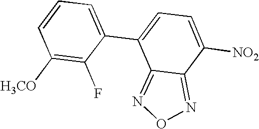 |
| 315 | 4-{4-[3-chloro-5-(trifluoromethyl)pyrindin- 2-yl]piperazin-1-yl}-7-nitro-2,1,3- benzoxadiazole | 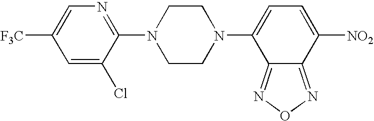 |
| 316 | methyl 4-({[6-(quinolin-2-ylthio)-2,1,3- benzoxadiazol-4- yl]sulfonyl}amino)benzoate | 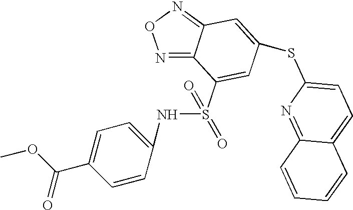 |
| 317 | 8-{[7-(4-phenyl-piperazin-1-sulfonamido)- 2,1,3-benzoxadiazol-4-yl]oxy}quinoline | 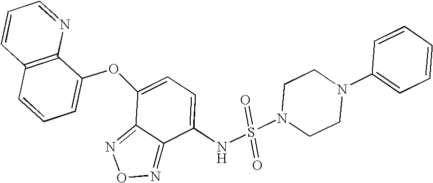 |
| 318 | 7-[4-(methylthio)phenyl]-4-nitro-2,1,3- benzoxadiazole | 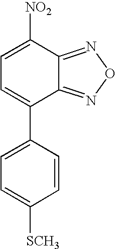 |
| 319 | 7-(3-methoxyphenyl)-4-nitro-2,1,3- benzoxadiazole | 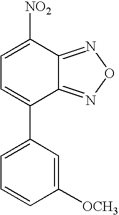 |
| 320 | 7-(3-fluorophenyl)-4-nitro-2,1,3- benzoxadiazole | 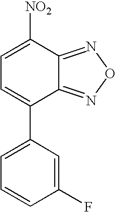 |
| 321 | 4-nitro-7-(quinolin-8-yl)-2,1,3- benzoxadiazole | 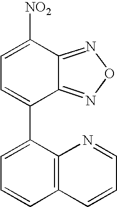 |
| 322 | 4-chloro-6-[3,5-(dimethyl)phenyl]-2,1,3- benzoxadiazole | 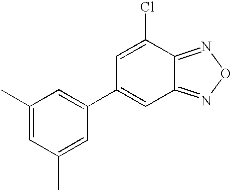 |
| 323 | 4,6-[3,5-(dimethoxy)phenyl]-2,1,3- benzoxadiazole | 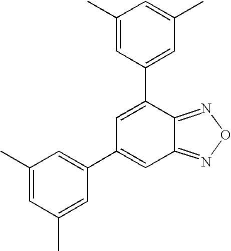 |
| 324 | 4-chloro-6-[3,5-(difluoro)phenyl]-2,1,3- benzoxadiazole | 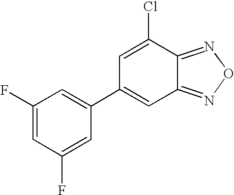 |
| 325 | 4-chloro-6-[3-(trifluoromethyl)phenyl]- 2,1,3-benzoxadiazole | 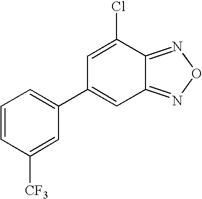 |
| 326 | 4-chloro-6-(3-methylphenyl)-2,1,3- benzoxadiazole | 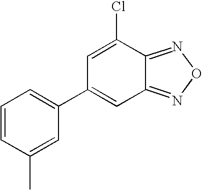 |
| 327 | 4-chloro-6-[4-(trifluoromethyl)phenyl]- 2,1,3-benzoxadiazole | 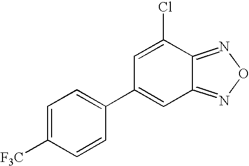 |
| 328 | 4-chloro-6-(3-methoxyphenyl)-2,1,3- benzoxadiazole | 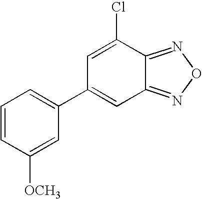 |
| 329 | 4-chloro-6-(4-(tert-butyl)phenyl)-2,1,3- benzoxadiazole | 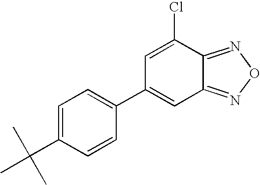 |
| 330 | 4-chloro-6-(2-methoxyphenyl)-2,1,3- benzoxadiazole | 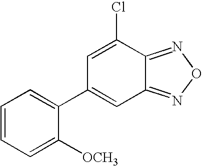 |
| 331 | 4-chloro-6-(3-nitrophenyl)-2,1,3- benzoxadiazole | 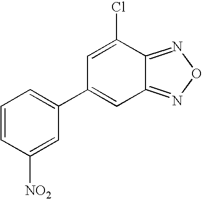 |
| 332 | 1′-{[7-chloro-(2,1,3-benzoxadiazol-4- yl)]sulfonyl}-3H-spiro[2-benzofuran-1,4′- piperidin]-3-one | 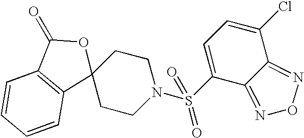 |
| 334 | 1′-{(7-[naphth-1-ylthio]-2,1,3- benzoxadiazol-4-yl)}sulfonyl-3H-spiro[2- benzofuran-1,4′-piperidine] | 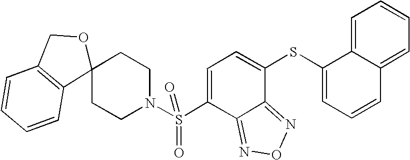 |
| 335 | methyl 4-{[(7-{[4-hydroxyphenyl]thio}- 2,1,3-benzoxadiazol-4- yl)sulfonyl]amino}benzoate | 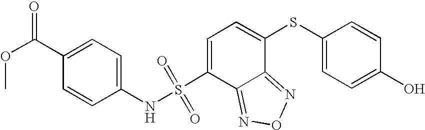 |
| 336 | 8-[(4-chloro-2,1,3-benzoxadiazol-6- yl)thio]quinoline | 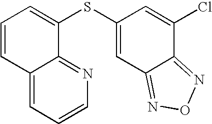 |
| 337 | 1′-{[5-chloro-(2,1,3-benzoxadiazol-4- yl)]sulfonyl}-3H-spiro[2-benzofuran-1,4′- piperidin]-3-one | 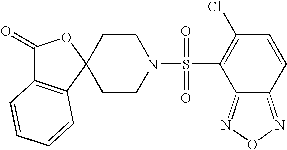 |
| 338 | 5-(1H-indazol-6-ylamino)-N-(1H-indazol- 6-yl)2,1,3-benzoxadiazole-4-sulfonamide | 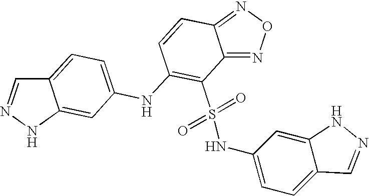 |
| 339 | 5-hydroxy-N-(1H-indazol-6-yl)2,1,3- benzoxadiazole-4-sulfonamide | 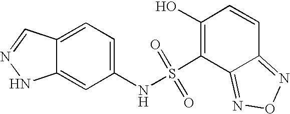 |
| 340 | 5-[4-(2- {trifluoromethyl}phenoxy)piperidin-1-yl]- 4-{[4-(2- {trifluoromethyl}phenoxy)piperidin-1- yl]sulfonyl}-2,1,3-benzoxadiazole | 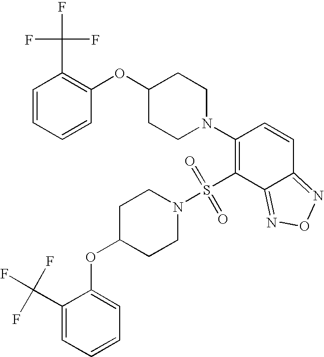 |
| 341 | 7-chloro-N-(4-chloro-2,5- dimethoxyphenyl)-2,1,3-benzoxadiazole-4- sulfonamide | 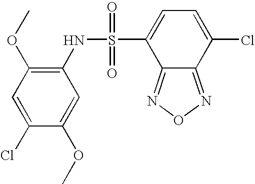 |
| 342 | N-(4-chloro-2,5-dimethoxyphenyl)-7-([4- chloro-2,5-dimethoxyphenyl]amino)-2,1,3- benzoxadiazole-4-sulfonamide | 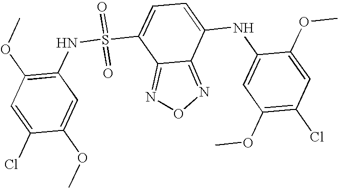 |
| 343 | 5-chloro-N-(4-chloro-2,5- dimethoxyphenyl)-2,1,3-benzoxadiazole-4- sulfonamide | 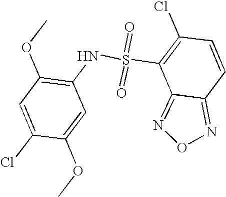 |
| 344 | N-(4-chloro-2,5-dimethoxyphenyl)-5-([4- chloro-2,5-dimethoxyphenyl]amino)-2,1,3- benzoxadiazole-4-sulfonamide | 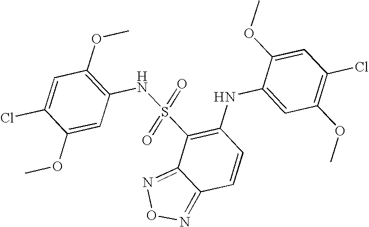 |
| 345 | 7-chloro-N-(2-fluorophenyl)-2,1,3- benzoxadiazole-4-sulfonamide | 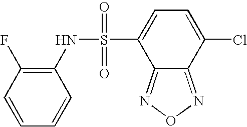 |
| 346 | N-(2-fluorophenyl)-7-(2- fluorophenylamino)-2,1,3-benzoxadiazole- 4-sulfonamide | 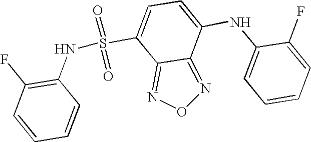 |
| 347 | 7-chloro-N-(2-isopropylphenyl)-2,1,3- benzoxadiazole-4-sulfonamide | 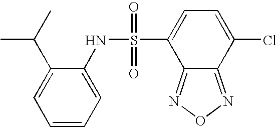 |
| 348 | N-(4-chloro-2,5-dimethoxyphenyl)-7- fluoro-2,1,3-benzoxadiazole-4-sulfonamide | 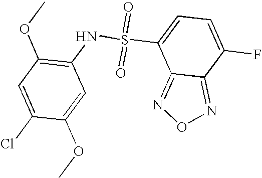 |
| 349 | N-(4-chloro-2,5-dimethoxyphenyl)-7-([4- chloro-2,5-dimethoxyphenyl]amino)-2,1,3- benzoxadiazole-4-sulfonamide | 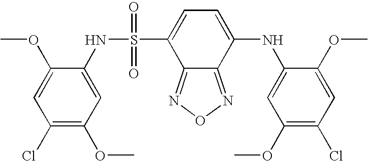 |
| 350 | 7-chloro-N-(2-methyl-3- [trifluoromethyl]phenyl)-2,1,3- benzoxadiazole-4-sulfonamide | 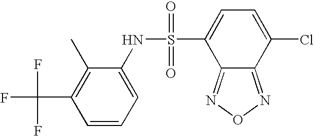 |
| 351 | 7-chloro-N-(5-fluoro-2-methylphenyl)- 2,1,3-benzoxadiazole-4-sulfonamide | 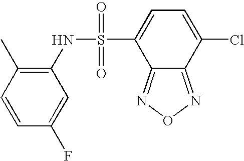 |
| 352 | 7-chloro-N-(2-isopropyl-6-methylphenyl)- 2,1,3-benzoxadiazole-4-sulfonamide | 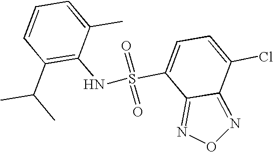 |
| 353 | 7-chloro-N-(2,6-dimethylphenyl)-2,1,3- benzoxadiazole-4-sulfonamide | 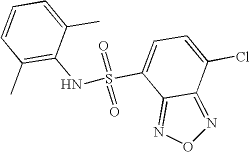 |
| 354 | 7-chloro-N-(2-chloro-4-methylphenyl)- 2,1,3-benzoxadiazole-4-sulfonamide | 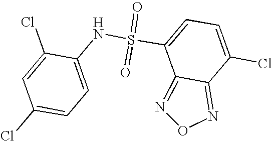 |
| 355 | 7-chloro-N-mesityl-2,1,3-benzoxadiazole- 4-sulfonamide | 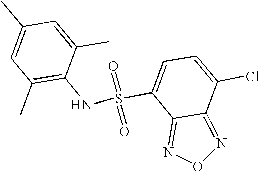 |
| 356 | 7-chloro-N-(2-methoxyphenyl)-2,1,3- benzoxadiazole-4-sulfonamide | 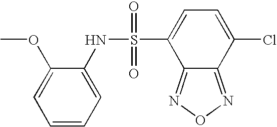 |
| 357 | 7-chloro-N-(2,4,6-trifluorophenyl)-2,1,3- benzoxadiazole-4-sulfonamide | 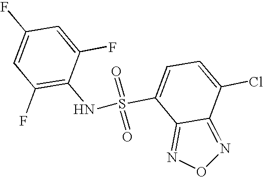 |
| 358 | 7-chloro-N-(4-fluoro-2-methylphenyl)- 2,1,3-benzoxadiazole-4-sulfonamide | 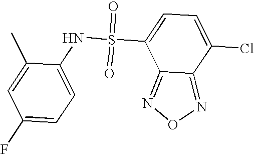 |
| 359 | 7-chloro-N-(2,5-dimethoxyphenyl)-2,1,3- benzoxadiazole-4-sulfonamide | 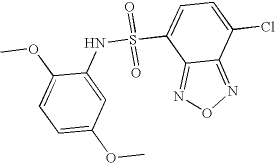 |
| 360 | methyl 4-chloro-2-{[(7-chloro-2,1,3- benzoxadiazol-4- yl)sulfonyl]amino}benzoate | 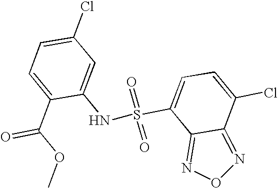 |
| 361 | methyl 4-chloro-2-(7-[N-{5-chloro-2- (methoxycarbonyl)phenyl}sulfamoyl]- 2,1,3-benzoxadiazol-4-ylamino)benzoate | 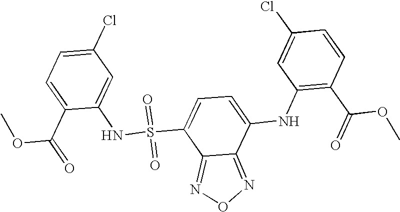 |
| 362 | 7-chloro-N-(4-chloro-2-methoxy-5- methylphenyl)-2,1,3-benzoxadiazole-4- sulfonamide | 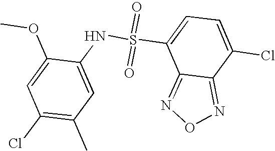 |
| 363 | 7-chloro-N-(1H-indol-5-yl)-2,1,3- benzoxadiazole-4-sulfonamide | 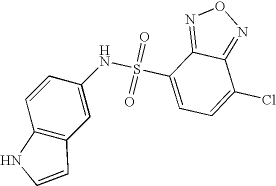 |
| 364 | 7-chloro-N-(2,4-difluorophenyl)-2,1,3- benzoxadiazole-4-sulfonamide | 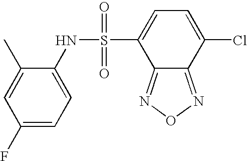 |
| 365 | 7-chloro-N-(2,5-dichlorophenyl)-2,1,3- benzoxadiazole-4-sulfonamide | 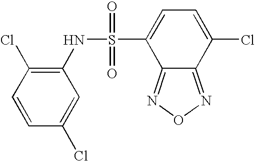 |
| 366 | 7-chloro-N-(2-[trifluoromethoxy]phenyl)- 2,1,3-benzoxadiazole-4-sulfonamide | 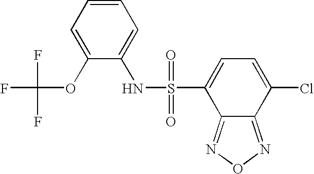 |
| 367 | N-(2,5-dimethoxyphenyl)-7-([2- methoxyphenyl]amino)-2,1,3- benzoxadiazole-4-sulfonamide | 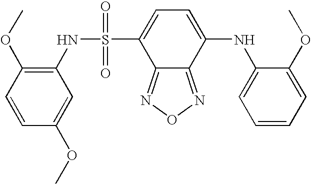 |
| 368 | 7-chloro-N-(5-methylisoxazol-3-yl)-2,1,3- benzoxadiazole-4-sulfonamide | 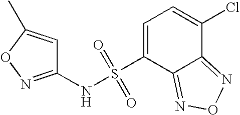 |
| 369 | N-(2,5-dimethoxyphenyl)-7-(morpholin-4- yl)-2,1,3-benzoxadiazole-4-sulfonamide | 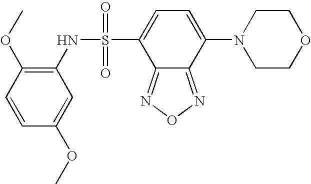 |
| 370 | 7-chloro-N-(2-methyl-1H-indol-5-yl)- 2,1,3-benzoxadiazole-4-sulfonamide | 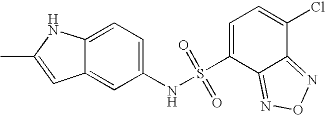 |
| 371 | N-(2,4-difluorophenyl)-7-([2,4- difluorophenyl]amino)-2,1,3- benzoxadiazole-4-sulfonamide | 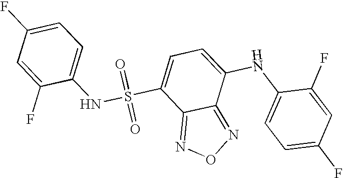 |
| 372 | N-(2-fluorophenyl)-7-(morpholin-4-yl)- 2,1,3-benzoxadiazole-4-sulfonamide | 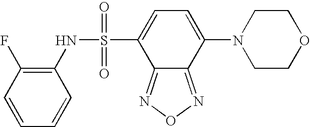 |
| 373 | 7-nitro-N-(2-[trifluoromethoxy]phenyl)- 2,1,3-benzoxadiazole-4-sulfonamide | 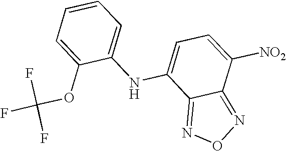 |
| 374 | 7-chloro-N-1,3-pyrimidin-4-yl-2,1,3- benzoxadiazole-4-sulfonamide | 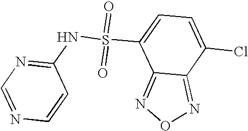 |
| 375 | 7-(morpholin-4-yl)-2,1,3-benzoxadiazol-4- amine | 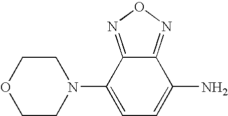 |
| 376 | N-(2,3-difluorophenyl)-7-(quinolin-8- ylthio)-2,1,3-benzoxadiazole-4- sulfonamide | 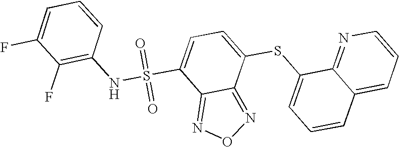 |
| 377 | N-(2,3-difluorophenyl)-7-(pyrimidin-4- ylthio)-2,1,3-benzoxadiazole-4- sulfonamide | 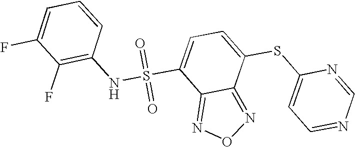 |
| 378 | N-(2,3-difluorophenyl)-7-([2- methoxyphenyl]thio)-2,1,3- benzoxadiazole-4-sulfonamide | 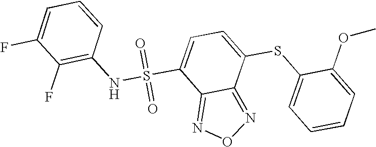 |
| 379 | N-(2,3-difluorophenyl)-7-(naphthalen-1- ylthio)-2,1,3-benzoxadiazole-4- sulfonamide | 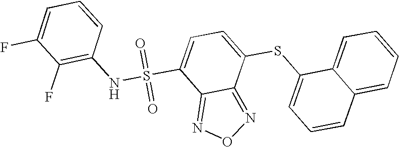 |
| 380 | N-(3-benzyloxypyridin-2-yl)-7-chloro- 2,1,3-benzoxadiazole-4-sulfonamide | 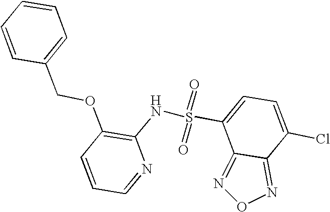 |
| 381 | 7-chloro-N-(2-methoxy-5-methylphenyl)- 2,1,3-benzoxadiazole-4-sulfonamide | 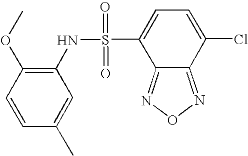 |
| 382 | N-(3-benzyloxypyridin-2-yl)-7- (morpholin-4-yl)-2,1,3-benzoxadiazole- 4-sulfonamide | 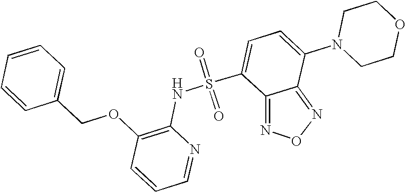 |
| 383 | 7-chloro-N-(7-hydroxynaphthalen-1-yl)- 2,1,3-benzoxadiazole-4-sulfonamide | 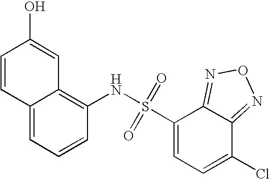 |
| 384 | N-(2-methoxy-5-methylphenyl)-7- (quinolin-8-ylthio)-2,1,3-benzoxadiazole- 4-sulfonamide | 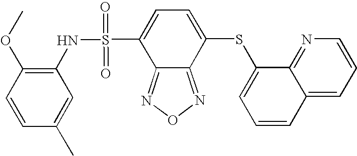 |
| 385 | N-(7-hydroxynaphthalen-1-yl)-7- (morpholin-4-yl)-2,1,3-benzoxadiazole-4- sulfonamide | 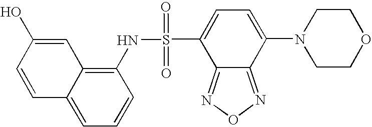 |
| 386 | N-(2-methoxy-4-methylphenyl)-7- (morpholin-4-yl)-2,1,3-benzoxadiazole-4- sulfonamide | 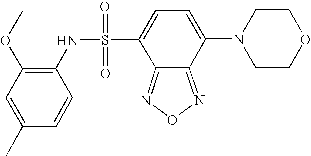 |
| 387 | N-(5-aminonaphthalen-1-yl)-7-chloro- 2,1,3-benzoxadiazole-4-sulfonamide | 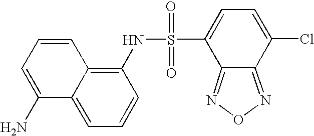 |
| 388 | 7-chloro-N-(4-nitronaphthalen-1-yl)-2,1,3- benzoxadiazole-4-sulfonamide | 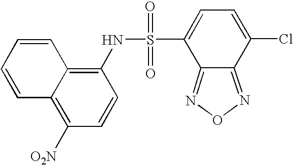 |
| 389 | N-(5-aminonaphthalen-1-yl)-7-(morpholin- 4-yl-2,1,3-benzoxadiazole-4-sulfonamide | 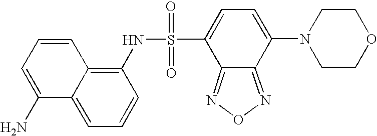 |
| 390 | N-(5-aminonaphthalen-1-yl)-7-(quinolin-8- ylthio)-2,1,3-benzoxadiazole-4- sulfonamide | 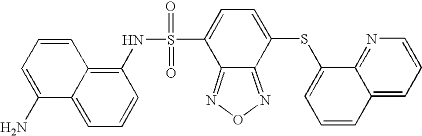 |
| 391 | 4-aminonaphthalen-1-yl 7-chloro-2,1,3- benzoxadiazole-4-sulfonate | 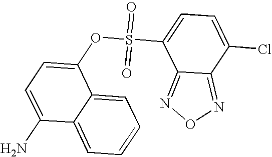 |
| 392 | 7-chloro-N-(naphthalen-1-yl)-2,1,3- benzoxadiazole-4-sulfonamide | 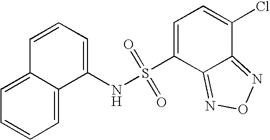 |
| 393 | 7-chloro-N-(4-hydroxynaphthalen-1-yl)- 2,1,3-benzoxadiazole-4-sulfonamide | 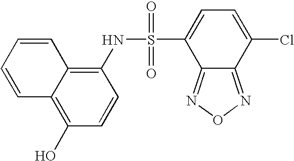 |
| 394 | 4-(7-chloro-2,1,3-benzoxadiazole-4- sulfonamido)naphthalen-1-yl 7-chloro- 2,1,3-benzoxadiazole-4-sulfonate | 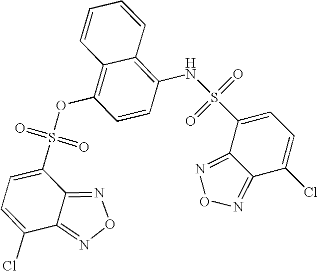 |
| 395 | N-(4-fluorophenyl)-7-(morpholin-4-yl)- 2,1,3-benzoxadiazole-4-sulfonamide | 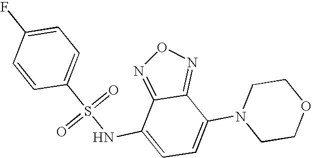 |
| 396 | 7-{(2,3-difluorophenyl)amino}-2,1,3- benzoxadiazol-4-amine | 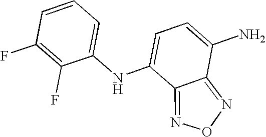 |
| 397 | N-(2,3-difluorophenyl)-7-nitro-2,1,3- benzoxadiazol-4-amine | 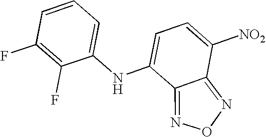 |
| 398 | N-(1H-indazol-5-yl)-2,1,3-benzoxadiazole- 4,7-diamine | 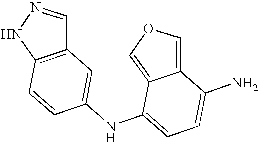 |
| 399 | 4-(7-chloro-2,1,3-benzoxadiazole-4- sulfonamido)benzoic acid | 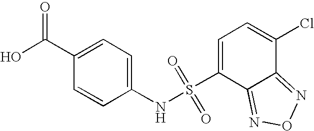 |
| 400 | 2-methoxy-N-(7-[{(2- trifluoromethoxy)phenyl}amino]-2,1,3- benzoxadiazol-4-yl)benzamide | 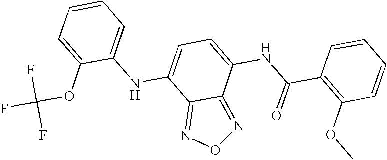 |
| 401 | N-(2-fluorophenyl)-7-nitro-2,1,3- benzoxadiazol-4-amine | 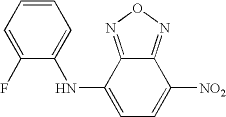 |
| 402 | N-(2,3-difluorophenyl)-7-([4- hydroxyphenyl]thio)-2,1,3- benzoxadiazole-4-sulfonamide | 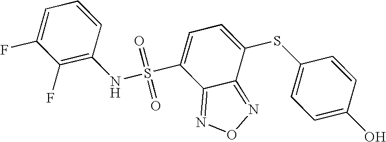 |
| 403 | N-(7-[{2,3-difluorophenyl}amino]-2,1,3- benzoxadiazol-4-yl)-2-methoxybenzamide | 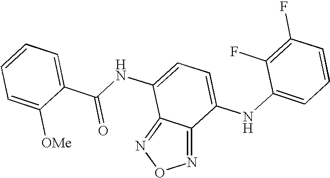 |
| 404 | N-(2-fluorophenyl)-7-([2- methoxyphenyl]amino)-2,1,3- benzoxadiazole-4-sulfonamide | 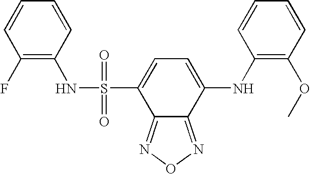 |
| 405 | 5,6-difluoro-4-(morpholin-4-yl)-2,1,3- benzoxadiazole | 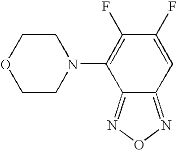 |
| 406 | 4,5-difluoro-6-(morpholin-4-yl)-2,1,3- benzoxadiazole | 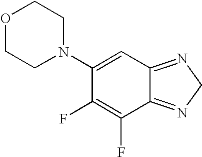 |
| 407 | N-(naphthalen-1-yl)-7-(quinolin-8-ylthio)- 2,1,3-benzoxadiazole-4-sulfonamide | 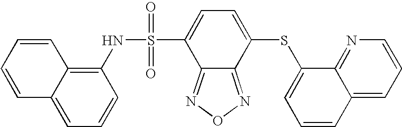 |
| 408 | N-(2,3-dimethoxyphenyl)-5-([2,3- dimethoxyphenyl]amino)-2,1,3- benzoxadiazole-4-sulfonamide | 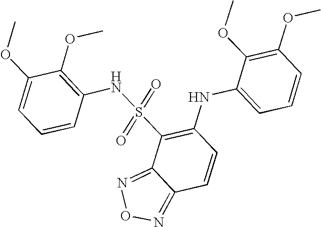 |
| 409 | 5-chloro-N-(2,3-dimethoxyphenyl)-2,1,3- benzoxadiazole-4-sulfonamide | 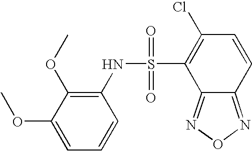 |
| 410 | 7-chloro-N-(2,3-dimethoxyphenyl)-2,1,3- benzoxadiazole-4-sulfonamide | 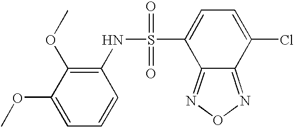 |
| 411 | 5,7-dichloro-4-{(morpholin-4-yl)sulfonyl}- 2,1,3-benzoxadiazole | 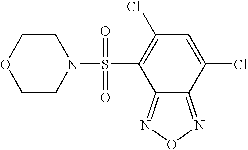 |
| 412 | 7-chloro-5-(morpholin-4-yl)-4- {(morpholin-4-yl)sulfonyl}-2,1,3- benzoxadiazole | 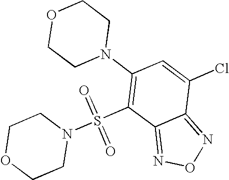 |
| 413 | N-(2,3-dimethoxyphenyl)-7-(naphthalen-1- ylthio)-2,1,3-benzoxadiazole-4- sulfonamide | 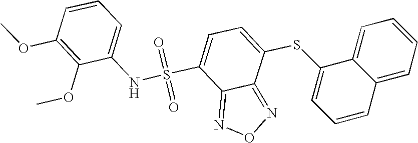 |
| 414 | 5,7-dichloro-N-(2,6-difluorophenyl)-2,1,3- benzoxadiazole-4-sulfonamide | 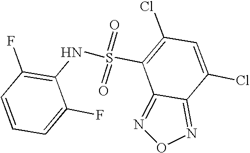 |
| 415 | 7-chloro-N-(naphthalen-1-yl)-5- (naphthalen-1-ylamino)-2,1,3- benzoxadiazole-4-sulfonamide | 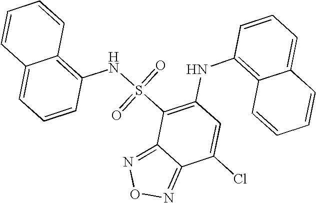 |
| 416 | 5,7-dichloro-N-(naphthalen-1-yl)-2,1,3- benzoxadiazole-4-sulfonamide | 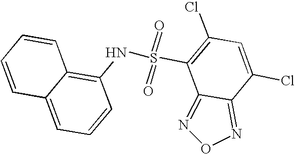 |
| 417 | 5-chloro-N-(naphthalen-1-yl)-7- (naphthalen-1-ylamino)-2,1,3- benzoxadiazole-4-sulfonamide | 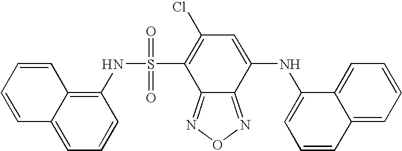 |
| 418 | 4,6-bis(3-[trifluoromethyl]phenyl)-2,1,3- benzoxadiazole | 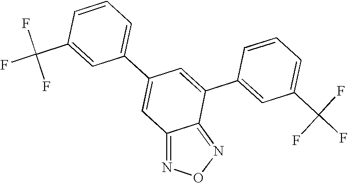 |
| 419 | 4,6-bis(3-methylphenyl)-2,1,3- benzoxadiazole | 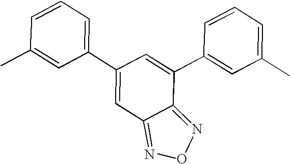 |
| 420 | 4,6-bis(4-[trifluoromethyl]phenyl)-2,1,3- benzoxadiazole | 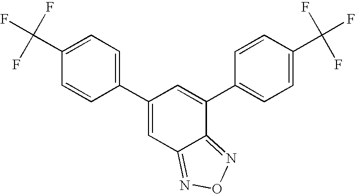 |
| 421 | 4,6-bis(3-methoxyphenyl)-2,1,3- benzoxadiazole | 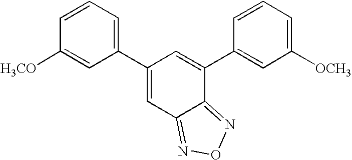 |
| 422 | 4,6-bis(4-t-butylphenyl)-2,1,3- benzoxadiazole | 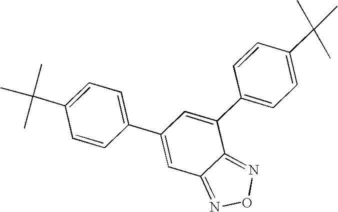 |
| 423 | 4,6-bis(2-methoxyphenyl)-2,1,3- benzoxadiazole | 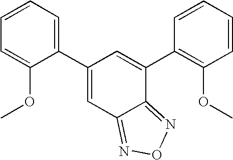 |
| 424 | N-(2-bromophenyl)-7-chloro-2,1,3- benzoxadiazole-4-sulfonamide | 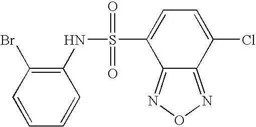 |
| 425 | 4-(quinolin-8-ylthio)naphtho[2,1- c][1,2,5]oxadiazol-7-ol | 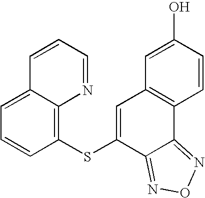 |
| 426 | 7-(3,5-dimethylphenyl)-2,1,3- benzoxadiazol-4-amine | 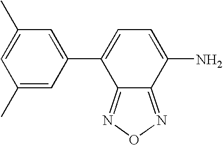 |
| 427 | 2,3-difluoro-N-(7-nitro-2,1,3- benzoxadiazol-4-yl)benzamide | 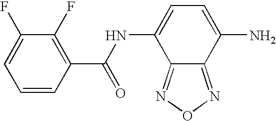 |
| 428 | 7-(3,5-difluorophenyl)-4-nitro-5- (piperidin-1-yl)-2,1,3-benzoxadiazole | 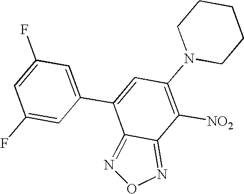 |
| 429 | 7-(3-[trifluoromethyl]phenyl)-4-nitro-5- (piperidin-1-yl)-2,1,3-benzoxadiazole | 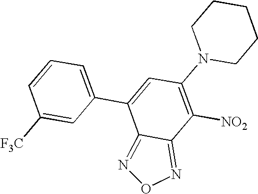 |
| 430 | 4-chloronaphtho[2,1-c][1,2,5]oxadiazol-7-ol | 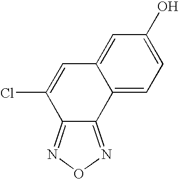 |
| 431 | 7-(4-[trifluoromethyl]phenyl)-4-nitro-5- (piperidin-1-yl)-2,1,3-benzoxadiazole | 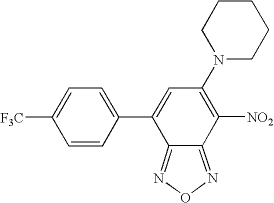 |
| 432 | 7-(3-methylphenyl)-4-nitro-5-(piperidin-1- yl)-2,1,3-benzoxadiazole | 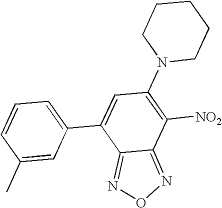 |
| 433 | 7-(4-t-butylphenyl)-4-nitro-5-(piperidin-1- yl)-2,1,3-benzoxadiazole | 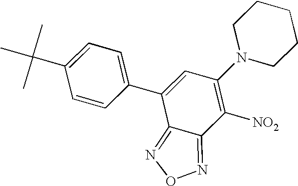 |
| 434 | 7-(3-methoxyphenyl)-4-nitro-5-(piperidin- 1-yl)-2,1,3-benzoxadiazole | 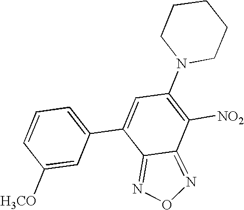 |
| 435 | 7-(4-t-butylphenyl)-2,1,3-benzoxadiazol-4- amine | 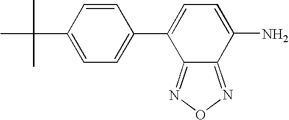 |
| 436 | N-(7-[3,5-dimethylphenyl]-2,1,3- benzoxadiazol-4-yl)acetamide | 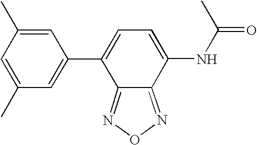 |
| 437 | 7-(2-fluorophenyl)-4-nitro-2,1,3- benzoxadiazole | 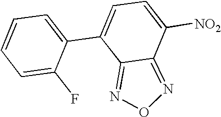 |
| 438 | 7-chloro-N-(2-methoxyethyl)-2,1,3- benzoxadiazole-4-sulfonamide | 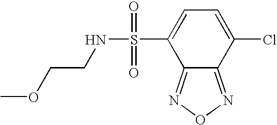 |
| 439 | 4-nitro-7-o-tolyl-2,1,3-benzoxadiazole | 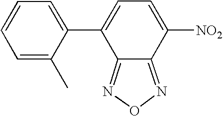 |
| 440 | 4-nitro-7-phenyl-2,1,3-benzoxadiazole | 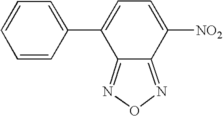 |
| 441 | 1-(4-[7-nitro-2,1,3-benzoxadiazol-4- yl]phenyl)ethanone | 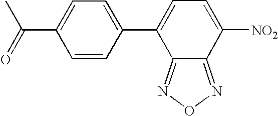 |
| 442 | 4-nitro-7-(4-fluorophenyl)-2,1,3- benzoxadiazole | 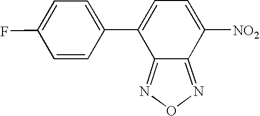 |
| 443 | 1-(3-[7-nitro-2,1,3-benzoxadiazol-4- yl]phenyl)ethanone | 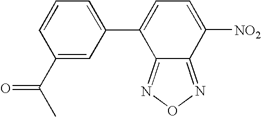 |
| 444 | 4-nitro-7-(3-nitrophenyl)-2,1,3- benzoxadiazole | 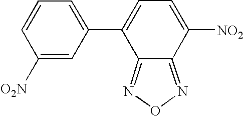 |
| 445 | 7-chloro-N-(3-phenylpropyl)-2,1,3- benzoxadiazole-4-sulfonamide | 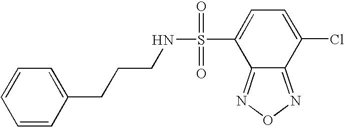 |
| 446 | 7-chloro-N-(pyridin-2-ylmethyl)-2,1,3- benzoxadiazole-4-sulfonamide | 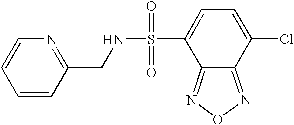 |
| 447 | 7-chloro-N-(cyclohexylmethyl)-2,1,3- benzoxadiazole-4-sulfonamide | 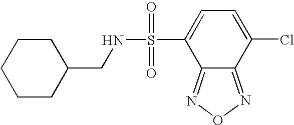 |
| 448 | 7-chloro-N-(cyclohexyl)-2,1,3- benzoxadiazole-4-sulfonamide | 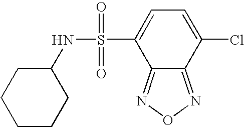 |
| 449 | 7-chloro-N-(cyclopropylmethyl)-2,1,3- benzoxadiazole-4-sulfonamide | 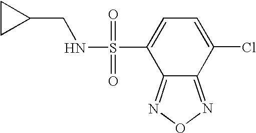 |
| 450 | 4-(3,5-dimethylphenyl)-5-methoxy-2,1,3- benzoxadiazole | 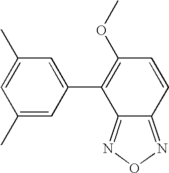 |
| 451 | 4-cyclohexenyl-7-nitro-2,1,3- benzoxadiazole | 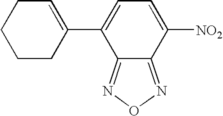 |
| 452 | N-(2-fluorophenyl)-5-methyl-2,1,3- benzthiadiazole-4-sulfonamide | 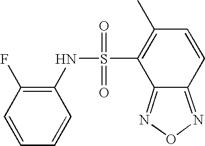 |
| 453 | N-(7-(4-t-butylphenyl)-2,1,3- benzoxadiazol-4-yl)acetamide | 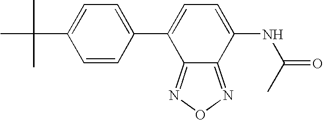 |
| 454 | 4-(4-t-butylphenyl)naphtho[2,1- c9 [1,2,5]oxadiazol-7-ol | 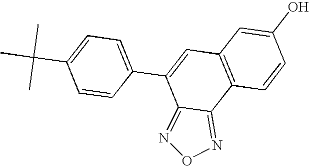 |
| 455 | 7-chloro-N-(pyridin-4-ylmethyl)-2,1,3- benzoxadiazole-4-sulfonamide | 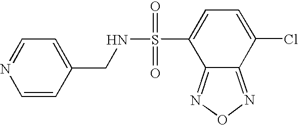 |
| 456 | 7-chloro-N-(3-[2-oxopyrrolidin-1- yl]propyl)-2,1,3-benzoxadiazole-4- sulfonamide | 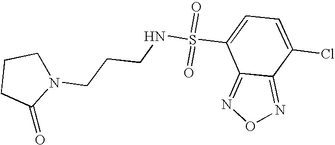 |
| 457 | 5-(2,3-dimethylphenyl)-6-fluoro-2,1,3- benzthiadiazole | 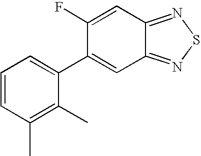 |
| 458 | 5-fluoro-6-phenyl-2,1,3-benzthiadiazole | 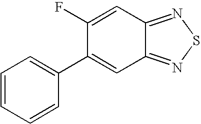 |
| 459 | 5-(3,4-dimethylphenyl)-6-fluoro-2,1,3- benzthiadiazole | 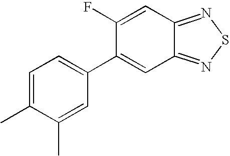 |
| 460 | 5-(benzodioxol-4-yl)-6-fluoro-2,1,3- benzthiadiazole | 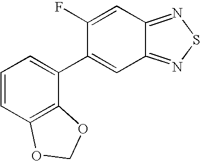 |
| 461 | 5-(3,5-dimethylphenyl)-6-fluoro-2,1,3- benzthiadiazole | 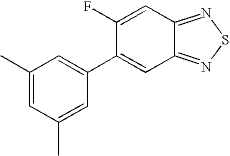 |
| 462 | 5-(4-methoxyphenyl)-6-fluoro-2,1,3- benzthiadiazole | 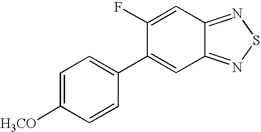 |
| 463 | 5-(3,4-dimethoxyphenyl)-6-fluoro-2,1,3- benzthiadiazole | 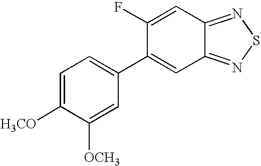 |
| 464 | 4-nitro-7-(4-phenoxyphenyl)-2,1,3- benzoxadiazole | 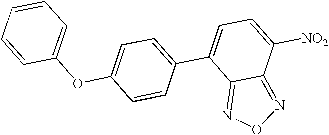 |
| 465 | 4-(3,4-dimethoxyphenyl)-7-nitro-2,1,3- benzoxadiazole | 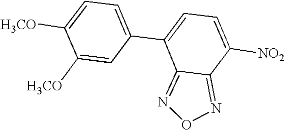 |
| 466 | 4-(4-methoxyphenyl)-7-nitro-2,1,3- benzoxadiazole | 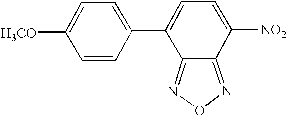 |
| 467 | 4-(benzodioxol-5-yl)-7-nitro-2,1,3- benzoxadiazole | 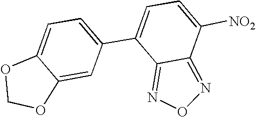 |
| 468 | 4-(4-methylphenyl)-7-nitro-2,1,3- benzoxadiazole | 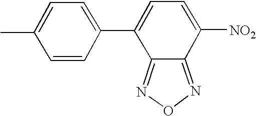 |
| 469 | 4-(naphthalen-1-yl)-7-nitro-2,1,3- benzoxadiazole | 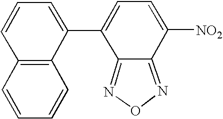 |
| 470 | 4-(2,3-dimethylphenyl)-7-nitro-2,1,3- benzoxadiazole | 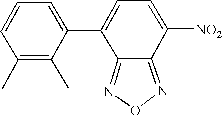 |
| 471 | 4-(2,5-dimethylphenyl)-7-nitro-2,1,3- benzoxadiazole | 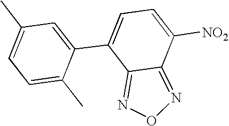 |
| 472 | 4-(3,4-dimethylphenyl)-7-nitro-2,1,3- benzoxadiazole | 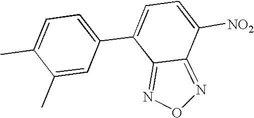 |
| 473 | 4-(2,3-dimethylphenyl)-6- (trifluoromethyl)-2,1,3-benzoxadiazole | 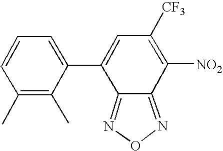 |
| 474 | 4-phenyl-6-(trifluoromethyl)-2,1,3- benzoxadiazole | 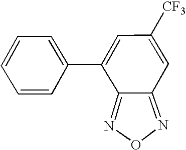 |
| 475 | 4-(benzodioxol-5-yl)-6-(trifluoromethyl)- 2,1,3-benzoxadiazole | 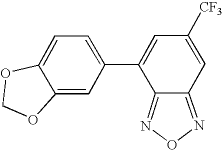 |
| 476 | 6-nitro-4-p-tolyl-2,1,3-benzoxadiazole | 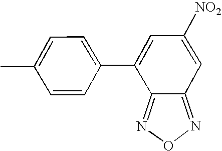 |
| 477 | 4-(2,4-bis[trifluoromethyl]phenyl)-6-nitro- 2,1,3-benzoxadiazole | 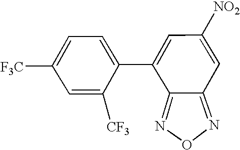 |
| 478 | 6-nitro-4-m-tolyl-2,1,3-benzoxadiazole | 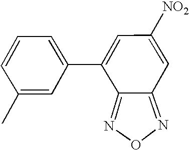 |
| 479 | 4-(3,5-difluorophenyl)-6-nitro-2,1,3- benzoxadiazole | 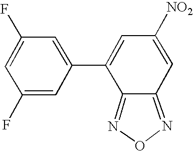 |
| 480 | 4-(3-[trifluoromethyl]phenyl)-6-nitro- 2,1,3-benzoxadiazole | 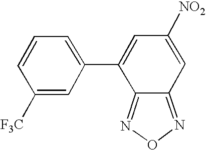 |
| 481 | 4-(4-t-butylphenyl)-6-nitro-2,1,3- benzoxadiazole | 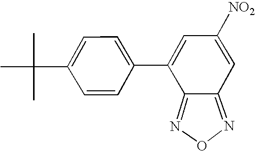 |
| 482 | 4-(2-methoxyphenyl)-6-nitro-2,1,3- benzoxadiazole | 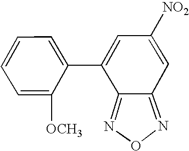 |
| 483 | 6-nitro-4-(quinolin-8-yl)-2,1,3- benzoxadiazole | 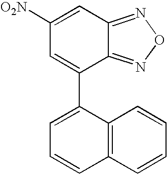 |
| 484 | 4-(2,5-dimethylphenyl)-6-nitro-2,1,3- benzoxadiazole | 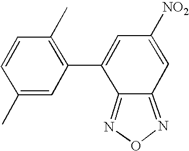 |
| 485 | 4-(4-chlorophenyl)-1-({7-(quinolin- 2ylthio)-2,1,3-benzoxadiazol-4- yl}sulfonyl)piperidin-4-ol | 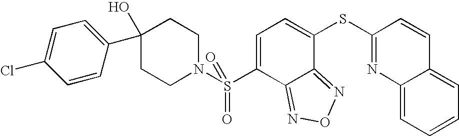 |
| 486 | N-(2,1,3-benzoxadiazol-4-yl)-1-(3- chloropyridin-2-yl) azetidine-3- carboxamide | 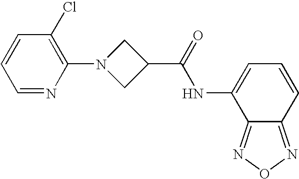 |

The compounds in the table above can be prepared using art recognized methods. All of the compounds in this application were named using ChemDraw Ultra version 8.0, which is available through Cambridgesoft.com, 100 Cambridge Park Drive, Cambridge, Mass. 02140, Namepro version 6.0, which is available from ACD Labs, 90 Adelaide Street West, Toronto, Ontario, M5H, 3V9, Canada, or were derived therefrom.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent group (e.g. CH₃—CH₂—), in certain circumstances a bivalent linking moiety can be “alkyl,” in which case those skilled in the art will understand the alkyl to be a divalent group (e.g., —CH₂CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for 0, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

The term “alkyl” as employed herein refers to straight and branched chain aliphatic groups having from 1 to 12 carbon atoms, preferably 1-8 carbon atoms, and more preferably 1-6 carbon atoms, which is optionally substituted with one, two or three substituents. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond.

The term “alkenyl” as used herein means an unsaturated straight or branched chain aliphatic group with one or more carbon-carbon double bonds, having from 2 to 12 carbon atoms, preferably 2-8 carbon atoms, and more preferably 2-6 carbon atoms, which is optionally substituted with one, two or three substituents. Preferred alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms, preferably 2-8 carbon atoms, and more preferably 2-6 carbon atoms, which is optionally substituted with one, two or three substituents. Preferred alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteroatom selected from the group consisting of O, S, and N.

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (C₁-C₆)alkyl(C₆-C₁₀)aryl, including, without limitation, benzyl, phenethyl, and naphthylmethyl. Further, the term “bis-aryl” group comprises an aryl group covalently linked to an aryl or heteroaryl group, any one of which may independently be optionally substituted or unsubstituted. Preferably, the bis-aryl group is (C₆-C₁₀)aryl(C₆-C₁₀)aryl or (C₆-C₁₀)aryl(C₆-C₁₀)heteroaryl, including, without limitation, 2,1,3-benzoxadiazolyl substituted in the benzo portion with phenyl or quinolinyl.

A “heterocyclic” group (or “heterocyclyl”) is an optionally substituted non-aromatic mono-, bi-, or tricyclic structure having from about 3 to about 14 atoms, wherein one or more atoms are selected from the group consisting of N, O, and S. One ring of a bicyclic heterocycle or two rings of a tricyclic heterocycle may be aromatic, as in indan and 9,10-dihydro anthracene. The heterocyclic group is optionally substituted on carbon with oxo or with one of the substituents listed above. The heterocyclic group is also optionally independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds an annular O or S atom is adjacent to another O or S atom.

As used herein, the term “heteroaryl” refers to optionally substituted groups having 5 to 14 ring atoms, preferably 5, 6, 9, or 10 ring atoms; having 6, 10, or 14 pi electrons shared in a cyclic array; and having, in addition to carbon atoms, between one or more heteroatoms selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzoftiryl, dibenzofuiryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

A “heteroaralkyl” or “heteroarylalkyl” group comprises a heteroaryl group covalently linked to an alkyl group, either of which is independently optionally substituted or unsubstituted. Preferred heteroalkyl groups comprise a C₁-C₆ alkyl group and a heteroaryl group having 5, 6, 9, or 10 ring atoms. Specifically excluded from the scope of this term are compounds having adjacent annular O and/or S atoms. Examples of preferred heteroaralkyl groups include pyridylmethyl, pyridylethyl, pyrrolylmethyl, pyrrolylethyl, imidazolylmethyl, imidazolylethyl, thiazolylmethyl, and thiazolylethyl.

An “arylene,” “heteroarylene,” or “heterocyclylene” group is an aryl, heteroaryl, or heterocyclyl group, as defined hereinabove, that is positioned between and serves to connect two other chemical groups.

Preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As employed herein, when a moiety (e.g., cycloalkyl, aryl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH-substituted with oxo is —C(O)—) nitro, alkyl, alkenyl, alkynyl, cycloalkyl, haloalkyl, haloalkenyl, haloalkynyl, halocycloalkyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, or any of these rings fused or spiro-fused to a cycloalkyl, heterocyclyl, aryl, or heteroaryl, wherein each of the foregoing is further optionally substituted with one more moieties listed in (a), above; and
(c) —(CH₂)₅—N(R³⁰)(R³¹), wherein s is from 0 (in which case the nitrogen is directly bonded to the moiety that is substituted) to 6, and R³⁰ and R³¹ are each independently hydrogen, cyano, oxo, carboxamido, amidino, C₁-C₈ hydroxyalkyl, C₁-C₃ alkylaryl, aryl-C₁-C₃ alkyl, C₁-C₈ alkyl, C₁-C₈ alkenyl, C₁-C₈ alkoxy, C₁-C₈ alkoxycarbonyl, aryloxycarbonyl, aryl-C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents from (a), above.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. For example, an optionally substituted phenyl includes, but not limited to, the following:

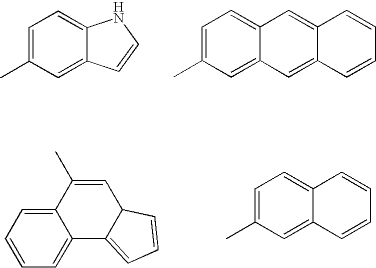

A “haloalkyl,” “haloalkenyl,” “haloalkynyl,” or “halocycloalkyl” is an alkyl, alkenyl, alkynyl, or cycloalkyl moiety in which from one to all hydrogens have been replaced with one or more halo.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

A moiety that is substituted is one in which one or more hydrogens have been independently replaced with another chemical substituent. As a non-limiting example, substituted phenyls include 2-flurophenyl, 3,4-dichlorophenyl, 3-chloro-4-fluoro-phenyl, 2-fluoro-3-propylphenyl. As another non-limiting example, substituted N-octyls include 2,4 dimethyl-5-ethyl-octyl and 3-cyclopentyl-octyl. Included within this definition are methylenes (—CH₂—) substituted with oxygen to form carbonyl —CO—).

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

Throughout the specification preferred embodiments of one or more chemical substituents are identified. Also preferred are combinations of preferred embodiments. Any compounds excluded from the scope of a particular genus of compounds of the invention (e.g., through phrases beginning “provided that when . . . ”) are intended to be excluded from all other genera of compounds.

Some compounds of the invention may have chiral centers and/or geometric isomeric centers (E- and Z- isomers), and it is to be understood that the invention encompasses all such optical, diastereoisomers and geometric isomers. The invention also comprises all tautomeric forms of the compounds disclosed herein.

“Atropisomers” are stereoisomers resulting from hindered rotation about single bonds where the barrier to rotation is high enough to allow for the isolation of the conformers (Eliel, E. L.; Wilen, S. H. Stereochemistry of Organic Compounds; Wiley & Sons: New York, 1994; Chapter 14, including pages 1150-1153 and the short definition on page 1193). Atropisomerism is significant because it introduces an element of chirality in the absence of stereogenic atoms. The invention is meant to encompass atropisomers, for example in cases of limited rotation around the single bonds emanating from the core 2,1,3-benzoxadiazole or 2,1,3-benzothiadiazole structure. Atropisomers are also possible and are also specifically included in the compounds and/or prodrugs of the invention.

Polymorphism in chemical substances is the ability of a single compound to exist in two or more solid phases, each having different arrangements and/or conformations of the individual molecules in the solid form (D. J. W. Grant, Theory and Origin of Polymorphism. In H. G. Brittain (ed.) Polymorphism in Pharmaceutical Solids. Marcel Dekker, Inc., New York, 1999, pp. 1-34). Generally, polymorphic solid forms can be crystalline or amorphous. Polymorphs of molecules or their solvates (for example, hydrates) can exist. Distinct polymorphic forms generally have different chemical and physical properties such as melting point, chemical reactivity, apparent solubility, dissolution rate, optical and electrical properties, vapor pressure, and density. The invention is meant to encompass in its scope, different polymorphic forms of the compounds of the invention.

The compounds of the invention may be administered in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any carboxy group in the compounds of this invention.

An in vivo hydrolyzable ester of a compound of the invention containing a hydroxy group includes inorganic esters such as phosphate esters and a-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4- position of the benzoyl ring. A suitable value for an in vivo hydrolyzable amide of a compound of the invention containing a carboxy group is, for example, a N—C₁₋₆-alkyl or N,N-di-C₁₋₆-alkyl amide such as N-methyl, N-ethyl, N-propyl, N,N-dimethyl, N-ethyl-N-methyl or N,N-diethyl amide.

### Pharmaceutical Compositions

In a second aspect, the invention provides pharmaceutical compositions comprising an inhibitor of histone deacetylase according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain preferred embodiments, compounds of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's The Science and Practice of Pharmacy, 20th Edition, 2000.

As used herein, the term pharmaceutically acceptable salts refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+W—, wherein R is R₃-R₇, and W is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compound is included in the pharmaceutically acceptable carrier or diluent in an amount sufficient to deliver to a patient a therapeutically effective amount without causing serious toxic effects in the patient treated. A preferred dose of the active compound for all of the above-mentioned conditions is in the range from about 0.01 to 300 mg/kg, preferably 0.1 to 100 mg/kg per day, more preferably 0.1 to about 50 mg per kilogram body weight of the recipient per day, and in some applications about 0.1 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

### Synthetic Schemes and Experimental Procedures

The compounds of the invention may be synthesized according to the methods known to those of ordinary skill in the art. For example, methods that may be used to make the compounds of the invention are described in Mallory, F. B. (Organic Syntheses, Coll. Vol. IV: pp 74-75 (John Wiley & Sons, 1963)); Smith, P. A. S. and Boyer, J. H. (Organic Syntheses, Coll. Vol. IV: pp 75-78 (John Wiley & Sons, 1963)), and in Can. J. Chem., pp 2482-2484 (1969). These references are incorporated by references in their entirety.

Scheme I outlines the general approach taken to synthesize the 2,1,3-benzoxadiazole scaffold. There are also commercially available 2,1,3-benzoxadiazoles and 2,1,3-benzothiadiazoles that can be used as starting materials for compounds of the invention. Schemes II-V outline some more specific synthetic methods used to make particular compounds of the invention.

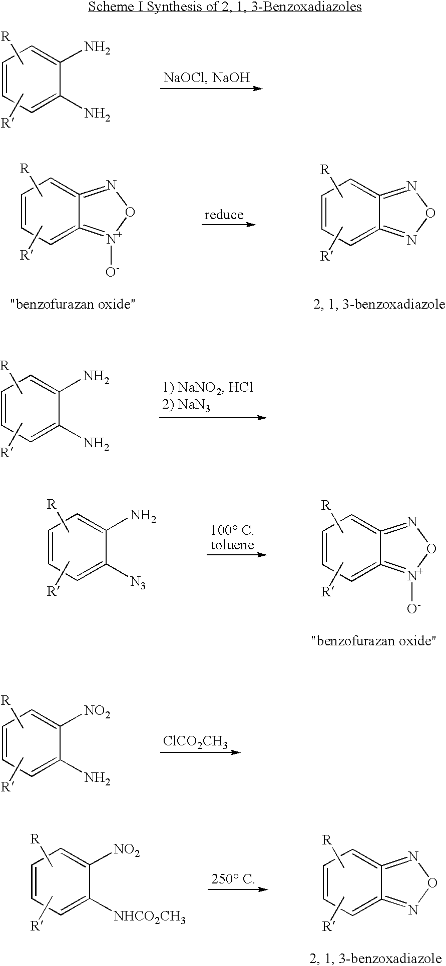

Referring to Scheme I, the 2,1,3-benzoxadiazole scaffold is prepared starting with a 1,2-dinitrogen substituted aryl system. In the first two reaction paths in Scheme I, a 1,2-bis aniline is used, for example. In the first reaction path, the 1,2-bis aniline is treated with NaOCl and NaOH in a ring closure reaction to give the corresponding benzofurazan oxide, which in turn is reduced to the corresponding 2,1,3-benzoxadiazole. Alternatively, the 1,2-bis aniline can be treated with NaNO₂/HCl (Sandmeyer reaction chemistry) to make an intermediate diazonium salt, which when treated with sodium azide (NaN₃) gives the corresponding ortho-amino phenyl azide. Heating the aryl azide in toluene gives the corresponding benzofurazan oxide, which, as outlined above, is reduced to the corresponding 2,1,3-benzoxadiazole. The final reaction path in Scheme I shows that an ortho-amino nitrobenzene can be converted to the corresponding 2,1,3-benzoxadiazole by first converting the amino group to a carbamate and then heating to high temperature.

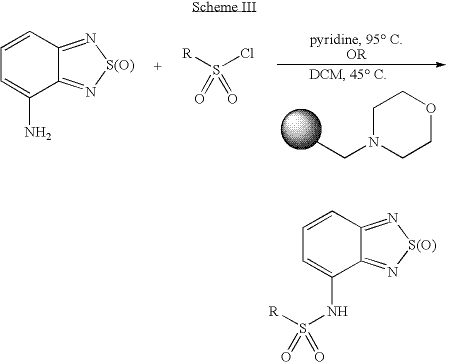

Scheme II outlines how compounds of the invention having a 4-sulfonamide group are made generally. A 4-amino-2,1,3-benzoxadiazole is treated with a desired sulfonyl chloride in the presence of an acid scavenger, preferably an amine base. Scheme II shows two preferred methods, one using pyridine as the base and heating to 95° C. to effect the reaction, and the other using a resin-bound morpholine as the base and heating to 45° C. to effect the reaction. Specific examples are described below and provide detailed synthetic procedures.

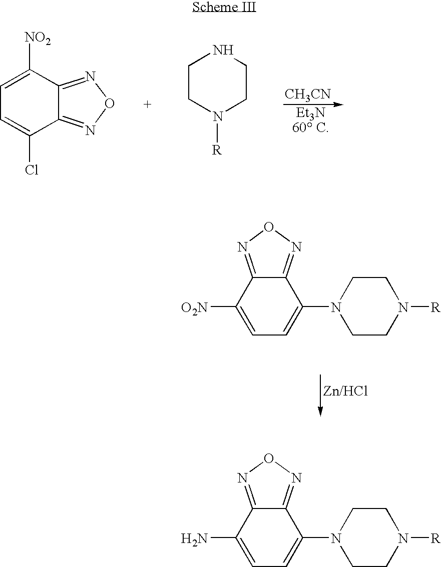

Scheme III outlines how compounds of the invention having a 4,7-di-nitrogen substitution are made generally. The appropriate 4-choro-7-nitro-2,1,3-benzoxadiazole is reacted with a primary or, as in this scheme, a secondary amine to give the addition adduct, which itself is a compound of the invention. Further derivatization at the 7-position is possible via reduction of the nitro group, for example with zinc in hydrochloric acid. The resulting primary amino group can be further derivitized for example via alkylation, acylation, sulfonylation (see Scheme II for example) and the like, as would be understood by one of ordinary skill in the art.

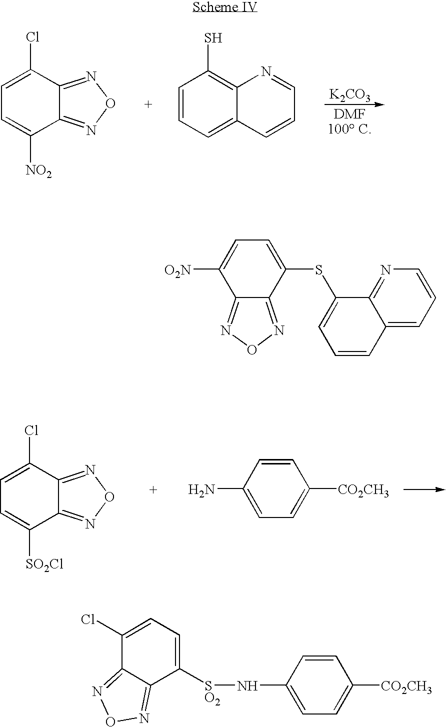

Scheme IV outlines how compounds of the invention having a 7-nitrogen-4-sulfur substitution are made generally. As depicted in the first reaction path, the appropriate 4-choro-7-nitro-2,1,3-benzoxadiazole is reacted with for example a sulfide to give the corresponding 7-nitrogen-4-sulfur containing analog. The sulfur group can be further oxidized to the corresponding sulfoxide or sulfone to make additional compounds of the invention. Alternatively, as depicted in the second reaction path, compounds of the invention having a substituted 7-chloro-4-sulfur substitution are made via reaction of the appropriate benzoxadiazole sulfonyl chloride with, for example, an amine partner to form sulfonamides and the like. In the example shown, the 7-choro group on the product (with sulfonamide installed) can further be exchanged via substitution reactions with thiols and amines for example (as shown in the specific examples) to make the corresponding 4-sulfur-7-sulfur-containing-2,1,3-benzoxadiazoles and 4-sulfur-7-nitrogen-containing-2,1,3-benzoxadiazoles, respectively.

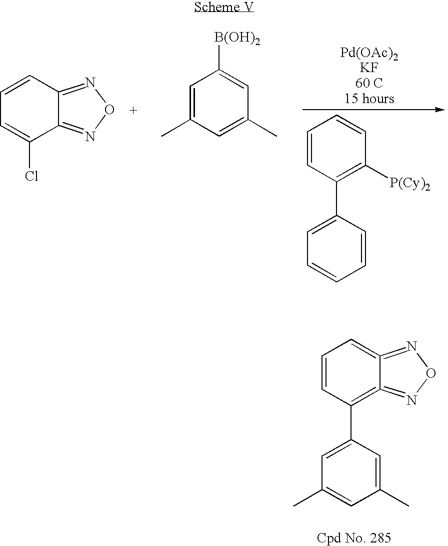

Scheme V outlines how compounds of the invention having, for example, a bis-aryl substitution are made generally. As depicted, the appropriate choro-2,1,3-benzoxadiazole is reacted with, for example, an aryl boronic acid under palladium-catalysis conditions to give the corresponding bis-aryl analog.

Schemes I-V, in conjunction with the examples described below, will make it sufficiently clear to one of ordinary skill in the art how to make the compounds of the invention. Compounds in Table 1 were made using the techniques described herein and were isolated and characterized by either ¹H-NMR, LC/MS or both. Commercially available starting materials, for example, amines, thiols, aryl halides, sulfonyl chlorides, boronic acids, were used in most cases along with the chemistry described to make the compounds of the invention.

### EXAMPLE 1

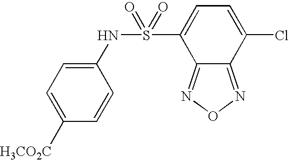

### Methyl 4-{[(7-chloro-2,1,3-benzoxadiazol-4-yl)sulfonyl]amino}benzoate (Cpd No. 8)

To 25.2 mg (0.1 m.mole) of 7-chloro-2,1,3-benzoxadiazole 4-sulfonylchloride was added 2 ml of dichoromethane and 50 mg of morpholino methyl polystyrene resin. The mixture was treated with 30.2 mg of methyl-p-amino benzoate. The mixture was shaken at room temperature for 5 hours at which time an examination of the reaction mixture by TLC indicated that all the sulfonyl chloride was consumed. The reaction was filtered and purified by radial silica-gel chromatography using 1:4 ethyl acetate:hexane as solvent to yield 34.8 mg (94.8% yield) of the desired product as a pale yellow solid.

¹H NMR (CDCl₃): δ 3.853 (s, 3H), 7.13 (d, 1H, J=8.5 Hz), 7.48 (d, 1H, J=8.5 Hz), 7.87 (d, 1H, J=6.9 Hz), 8.0 (d, 1H, J=6.9 Hz); LC/MS purity 100%. MS: M+1 and M-1 seen.

### EXAMPLE 2

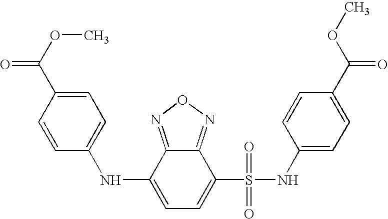

### Methyl 4-{[(7-{[4-(methoxycarbonyl)phenyl]amino}-2,1,3-benzoxadiazol-4-yl)sulfonyl]amino}benzoate (Cpd No. 9)

To 22 mg (0.877 m.mol) of methyl 4-{[(7-chloro-2,1,3-benzoxadiazol-4-yl)sulfonyl]amino}benzoate was added 1 ml of ethanol and 30 mg of methyl-p-amino benzoate and 1 drop of triethyl amine. The mixture was heated in a microwave reactor at 160° C. for 2 hours. The reaction was about 75% complete at this time. The heating was continued for a further 1 hour at which time there was no trace of starting material. The solvent was then evaporated and the residue purified using radial silica-gel chromatography using 1:4 EtOAc:hexane as solvent. The reaction yielded 32.5 mg 77% yield of the desired product.

¹H NMR (CDCl₃): δ 3.84 (s, 3H), 3.94 (s, 3H); 6.88 (d, 1H, J=7.8 Hz); 7.13 (d, 2H, J=8.7 Hz); 7.35 (d, 2H, J=8.7 Hz); 7.42 (s, 1H); 7.85 (d, 2H, J=8.7 Hz); 7.98 (d, 1H, J=7.8 Hz); 8.1 (d, 2H, J=8.7 Hz); LC/MS purity 100%. MS 481 (M−1) seen.

### EXAMPLE 3

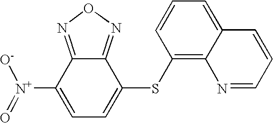

### 8-[(7-Nitro-2,1,3-benzoxadiazol-4-yl)thio]quinoline (Cpd No. 5)

To 40 mg (0.2 mmol) of 4-chloro-7-nitro-2,1,3-benzoxadiazole was added 2 ml of DMF and 60 mg of potassium carbonate. To this reaction mixture was added 128 mg (4 equivalents) of 8-mercaptoquinoline. The reaction turned bright red. The reaction mixture was heated at 60° C. for 4 hrs. Examination of the reaction by TLC showed no starting material. The reaction mixture was pored into 100 ml of water and extracted with ethyl acetate and methylene chloride. The combined organic layers were dried and the solvent evaporated. The residue was purified by column chromatography on silica gel using 1:2 EtOAc:Hexane as eluant. The 8-[(7-nitro-2,1,3-benzoxadiazol-4-yl)thio]quinoline was obtained as a red solid. 48.9 mg (75.4% yield)

¹H NMR (CDCl₃): δ 6.45 (d, 1H, J=7.8 Hz); 7.55 (m, 1H); 7.7 (dd, 1H, J=7.8 and 1.5 Hz); 8.1 (dd, 2H, J=1.5 and 7.8 Hz); 8.2 (dd, 1H, J=1.5 and 7.8 Hz); 8.3 (dd, 1H, J=1.5 and 7.8 Hz); 8.95 (dd, J=1.5 and 7.8 Hz); LC/MS purity 100%. MS 325 (M+1) seen.

### EXAMPLE 4

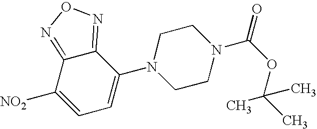

### Tert-butyl 4-(7-nitro-2,1,3-benzoxadiazol-4-yl)piperazine-1-carboxylate (Cpd No. 72)

To 20 mg (0.1 mmol) of 4-chloro-7-nitro-2,1,3-benzoxadiazole was added 2 ml of acetonitrile and 1 drop of triethyl amine. The mixture was then treated with 45 mg of 1-Boc piperazine. The reaction was heated with shaking at 60° C. for 4 hours. The reaction was complete at this time. The solvent was evaporated and the residue purified by radial silica gel chromatography using 1:4 EtOAc:hexane as solvent. The product was obtained in 79% yield (27.5 mg).

¹H NMR (CDCl₃): δ 1.510 (s, 9H); 3.7 (m, 4H); 4.1 (brs, 4H); 6.27 (d, 1H, J=8.7 Hz); 8.4 (d, 1H, 8.7 Hz); LC/MS purity 95%. MS 350.25 (M+1) seen.

### EXAMPLE 5

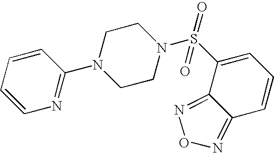

### 4-[(4-Pyridin-2-ylpiperazin-1-yl)-sulfonyl]-2,1,3-benzoxadiazole (Cpd No. 123)

To 30.8 mg (0.2 m.mol) of 4-chloro benzofurazan was added 1.5 ml of ethanol and 2 drops of triethylamine. The reaction mizture was treated with 50 mg of 1-(2-pyridyl)-piperazine and the reaction heated in a microwave reactor at 180° C. for 2 hours. Reaction was about 30% complete. The reaction was further heated at 180° C. for 4 more hours at which time it was nearly complete. The reaction mixture was passed through a plug of silica and then purified by radial silica gel chromatography using 1:4 EtOAc:hexane as solvent. Yield of product was 34.2 mg (60%)

¹H NMR (CDCl₃): δ 3.9 (m, 4H); 4.3 (m, 4H); 6.32 (d, 1H, J=8.7 Hz); 6.65 (dd, 1H, J=0.3 and 8.7 Hz); 6.72 (dd, 1H, J=1.8 and 5.1 Hz); 7.55 (dt, 2H, J=0.3 and 8.7 Hz); 8.21 (dd, J=1.8 and 5.1 Hz); 8.44 (d, J=8.7 Hz); LC/MS purity 97%. MS 327 (M+1) seen.

### EXAMPLE 6

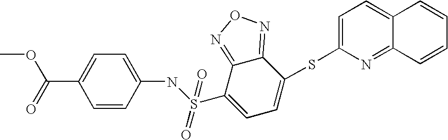

### Methyl 4-({[7-(quinolin-2-ylthio)-2,1,3-benzoxadiazol-4-yl]sulfonyl}amino)benzoate (Cpd No. 316)

To 10 mg of methyl 4-{[(7-chloro-2,1,3-benzoxadiazol-4-yl)sulfonyl]amino}benzoate (0.027 mmol) was added 1.5 ml of acetonitrile and 30 mg of 2-thio-quinoline and 1 drop of triethylamine. The reaction mixture was heated at 60° C. overnight. The reaction mixture was concentrated and purified using radial silica-gel chromatography using 1:4 to 100% ethyl acetate as solvent. The pure product was obtained in 40% yield (5.3 mg)

¹H NMR (CDCl₃): δ 3.856 (s, 3H); 7.15 (d, 2H, J=9 Hz); 7.45 (d, 1H, J=8.7 Hz); 7.5 (m, 2H); 7.7 (m, 1H); 7.79 (m, 3H); 7.88 (d, 2H, J=8.7 Hz); 7.99 (d, 1H, J=7.2 Hz); 8.12 (d, 1H, J=8.7 Hz); LC/MS purity 95%. MS 493.19 (M+1) and 491.19 (M−1) seen.

### EXAMPLE 7

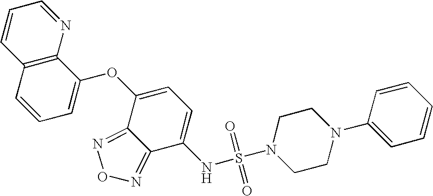

### 8-{[7-(4-Phenyl-piperazin-1-sulfonamido)-2,1,3-benzoxadiazol-4-yl]oxy}quinoline (Cpd No. 317)

To 7-chloro-{1-phenyl-(4-sulfonamido)-piperazinyl}-2,1,3-benzoxadiazole 10 mg (0.026 mmol) was added 2 ml of ethanol and 1 drop of triethylamine. To this reaction mixture was added 50 mg of 8-hydroxy quinoline. The reaction was heated in a microwave reactor for 2 hours at 160° C. The reaction was complete. The product was purified using radial silica-gel chromatography using 1:1 to 100% EtOAc:hexane as solvent. Product was obtained in 65% yield. (8.2 mg).

¹H NMR (CDCl₃): δ 6.32 (d, 1H, J=7.5 Hz); 6.88 (dd, 2H, J=0.3 and 8.1 Hz); 7.48 (dd, 1H, J=4.5 Hz and 8.4 Hz); 7.6 (d, 2H, J=4.5 Hz); 7.8 (dd, 2H, J=0.3 and 8.1 Hz); 8.26 (dd, 1H, J=1.5 and 8.1 Hz); 8.80 (dd, 1H, J=0.3 and 4.2 Hz); LC/MS purity 92%. MS 488.79 (M+1) seen.

### EXAMPLE 8

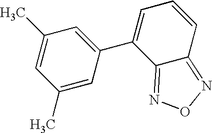

### 4-(3,5-Dimethylphenyl)-2,1,3-benzoxadiazole (Cpd. no. 285)¹

A vial containing a suspension of 4-chloro-2,1,3-benzoxadiazole (77 mg, 0.5 mmol), 3,5-dimethylphenylboronic acid (105 mg, 0.7 mmol), potassium fluoride (87 mg, 1.5 mmol), palladium (II) acetate (3 mg, 0.005 mmol), and 2-(dicyclohexylphosphino)biphenyl (3.5 mg, 0.01 mmol) in toluene (3 mL) was twice degassed and back-filled with argon and capped. The reaction mixture was heated at 60 C for 15 hours, cooled, diluted with ethyl acetate, washed successively with 1N NaOH and brine, and dried over MgSO₄. The organic layer was filtered through a plug of celite and silica gel and eluted with ethyl acetate. The filtrate was concentrated and the residue was purified by radial silica gel chromatography to afford Cpd No. 285 (54 mg, 48%).

¹H NMR (CDCl₃, 300 MHz) δ 7.77 (d, J=8.7 Hz, 1H), 7.58 (br s, 2H), 7.55-7.42 (m, 2H), 7.10 (s, 1H) 2.43 (s, 6H); LCMS purity: 100%. MS (positive ion): Found 225 (MH⁺).

¹Reference for procedure: J. Am. Chem. Soc. 1999, 121, 9550.

### EXAMPLE 9

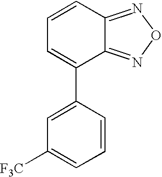

### 4-[3-(Trifluoromethyl)phenyl]-2,1,3-benzoxadiazole (Cpd. No. 305)

This compound was prepared according to the procedure for the preparation of Cpd. No. 285 and was purified by reverse phase liquid chromatography.

¹H NMR (CDCl₃, 300 MHz) δ 8.24 (m, 1H), 8.21 (m, 1H), 7.87 (, d, J=9 Hz, 1H), 7,74-7.65 (m, 2H), 7.64 (d, J=6.9 Hz, 1H), 7.56-7.51 (m, 1H).; MS (negative ion): Found 263 (M−H+).

### EXAMPLE 10

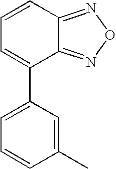

### 4-(3-Methylphenyl)-2,1,3-benzoxadiazole (Cpd. No. 306)

This compound was prepared according to the procedure for the preparation of Cpd. No. 285 and was purified by reverse phase liquid chromatography.

¹H NMR (CDCl₃, 300 MHz) δ 7.80-7.76 (m, 3H), 7.57-7.46 (m, 2H), 7.41 (app t, J=14.4 Hz, 1H), 7.29-7.25 (m, 1H), 2.48 (s, 3H); MS (negative ion): Found 209 (M−H⁺).

### EXAMPLE 11

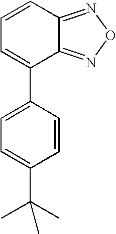

### 4-(4-Tert-butylphenyl)-2,1,3-benzoxadiazole (Cpd. No. 307)

This compound was prepared according to the procedure for the preparation of Cpd. No. 285 and was purified by reverse phase liquid chromatography.

¹H NMR (CDCl₃, 300 MHz) δ 7.93 (d, J=6.6 Hz, 2H), 7.78 (d, J=8.7 Hz, 1H), 7.57-7.46 (m, 4H), 1.40 (s, 9H); MS (negative ion): Found 251 (M−H+).

### EXAMPLE 12

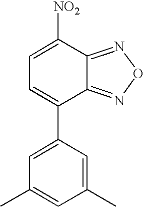

### 4-(3,5-Dimethylphenyl)-7-nitro-2,1,3-benzoxadiazole (Cpd. No. 286)

This compound was prepared according to the procedure for the preparation of Cpd. No. 285 and was purified by reverse phase liquid chromatography.

¹H NMR (CDCl₃, 300 MHz) δ 8.56 (d, J=7.8 Hz, 1H), 7.71 (d, J=7.5 Hz, 1H), 7.64 (s, 2H), 7.22 (m, 1H), 2.46 (s, 6H); MS (negative ion): Found 269 (M).

### EXAMPLE 13

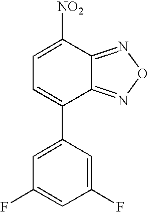

### 4-[(3,5-Difluoro)phenyl]-7-nitro-2,1,3-benzoxadiazole (Cpd. No. 308)

This compound was prepared according to the procedure for the preparation of Cpd. No. 285 and was purified by reverse phase liquid chromatography.

¹H NMR (CDCl₃, 300 MHz) δ 8.58 (d, J=7.5 Hz, 1H), 7.78 (d, J=7.9 Hz, 1H), 7.66-7.59 (m, 2H), 7.04 (t, J=2.18.7 Hz, 1H); MS (negative ion): Found 277 (M).

### EXAMPLE 14

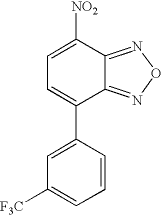

### 4-Nitro-7-[3-(trifluoromethyl)phenyl]-2,1,3-benzoxadiazole (Cpd. No. 309)

This compound was prepared according to the procedure for the preparation of Cpd. No. 285 and was purified by reverse phase liquid chromatography.

¹H NMR (CDCl₃, 300 MHz) δ 8.61 (d, J=7.8 Hz, 1H), 8.28-8.26 (m, 2H), 7.86-7.72 (m, 3H); MS (negative ion): Found 309 (M).

### EXAMPLE 15

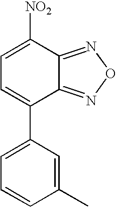

### 4-(3-Methylphenyl)-7-nitro-2,1,3-benzoxadiazole (Cpd. No. 310)

This compound was prepared according to the procedure for the preparation of Cpd. No. 285 and was purified by reverse phase liquid chromatography.

¹H NMR (CDCl₃, 300 MHz) δ 8.58 (d, J=7.8 Hz, 1H), 7.85-7.83 (m, 2H), 7.73 (, d, J=7.5 Hz, 1H), 7.48 (app t, J=7.8 Hz, 1H), 7.40-7.38 (m, 1H), 2.51 (s, 3H); MS (negative ion): Found 255 (M).

### EXAMPLE 16

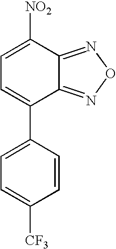

### 4-Nitro-7-[4-(trifluoromethyl)phenyl]-2,1,3-benzoxadiazole (Cpd. No. 311)

This compound was prepared according to the procedure for the preparation of Cpd. No. 285 and was purified by reverse phase liquid chromatography.

¹H NMR (CDCl₃, 300 MHz) δ 8.61 (d, J=7.5 Hz, 1H), 8.16 (d, J=8.7 Hz, 1H), 7.87-7.80 (m, 3H); MS (negative ion): Found 309 (M).

### EXAMPLE 17

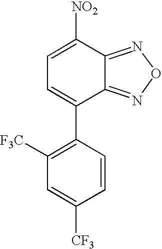

### 4-[2,4-Bis(trifluoromethyl)phenyl]-7-nitro-2,1,3-benzoxadiazole (Cpd. No. 312)

This compound was prepared according to the procedure for the preparation of Cpd. No. 285 and was purified by reverse phase liquid chromatography.

¹H NMR (CDCl₃, 300 MHz) δ 8.58 (d, J=7.5 Hz, 1H), 8.15 (s, 1H), 8.00 (d, J=8.1 Hz, 1H), 7.67 (d, J=8.1 Hz, 1H), 7.58 (d, J=7.2 Hz, 1H); MS (negative ion): Found 377 (M).

### EXAMPLE 18

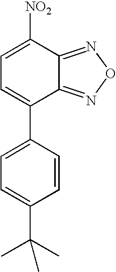

### 4-(4-Tert-butylphenyl)-7-nitro-2,1,3-benzoxadiazole (Cpd. No. 313)

This compound was prepared according to the procedure for the preparation of Cpd. No. 285 and was purified by reverse phase liquid chromatography.

¹H NMR (CDCl₃, 300 MHz) δ 8.58 (d, J=7.8 Hz, 1H), 8.01 (d, J=8.4 Hz, 2H), 7.73 (d, J=7.8 Hz, 1H), 7.61 (d, J=8.1 Hz, 2H), 1,41 (s, 9H); MS (negative ion): Found 297 (M).

### EXAMPLE 19

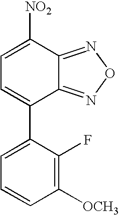

### 4-(2-Fluoro-3-methoxyphenyl)-7-nitro-2,1,3-benzoxadiazole (Cpd. No. 314)

This compound was prepared according to the procedure for the preparation of Cpd. No. 285 and was purified by reverse phase liquid chromatography.

¹H NMR (CDCl₃, 300 MHz) δ 8.58 (d, J=7.8 Hz, 1H), 7.79 (d, J=7.8 Hz, 1H), 7.44-7.40 (m, 1H), 7.28-7.25 (m, 1H), 7.19-7.09 (m, 1H), 3.98 (s, 3H).

### EXAMPLE 20

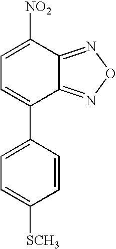

### 7-[4-(Methylthio)phenyl]-4-nitro-2,1,3-benzoxadiazole (Cpd No. 318)

This compound was prepared according to the procedure for the preparation of Cpd No. 285 and was purified by reverse phase liquid chromatography.

¹H NMR (CDCl₃, 300 MHz) δ 8.57 (d, J=7.8 Hz, 1H), 8.03 (d, J=8.4 Hz, 2H), 7.72 (d, J=7.5 Hz, 1H), 7.41 (d, J=8.4 Hz, 2H), 2.58 (s, 3H); MS (positive ion): Found 288 (MH⁺).

### EXAMPLE 21

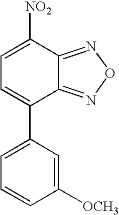

### 7-(3-Methoxyphenyl)-4-nitro-2,1,3-benzoxadiazole (Cpd No. 319)

This compound was prepared according to the procedure for the preparation of Cpd No. 285 and was purified by reverse phase liquid chromatography.

¹H NMR (CDCl₃, 300 MHz) δ 8.57 (d, J=7.8 Hz, 1H), 7.75 (d, J=7.8 Hz, 1H), 7.62-7.59 (m, 2H), 7.50 (t, J=8.1 Hz, 1H), 7.14-7.11 (m, 1H), 3.92 (s, 3H); MS (positive ion): Found 272 (MH⁺).

### EXAMPLE 22

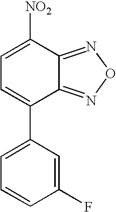

### 7-(3-Fluorophenyl)-4-nitro-2,1,3-benzoxadiazole (Cpd No. 320)

This compound was prepared according to the procedure for the preparation of Cpd No. 285 and was purified by reverse phase liquid chromatography.

¹H NMR (CDCl₃, 300 MHz) δ 8.59 (d, J=7.8 Hz, 1H), 7.87-7.84 (m, 1H), 7.88 (dt, J=2.4, 9.6 Hz, 1H), 7.77 (d, J=7.5 Hz, 1H), 7.61-7.54 (m, 1H), 7.29 (app ddt, J=0.6, 2.4, 7.5 Hz, 1H); MS (positive ion): Found 260 (MH⁺).

### EXAMPLE 23

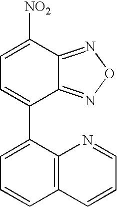

### 4-Nitro-7-(quinolin-8-yl)-2,1,3-benzoxadiazole (Cpd No. 321)

This compound was prepared according to the procedure for the preparation of Cpd No. 285 and was purified by reverse phase liquid chromatography.

¹H NMR (CDCl₃, 300 MHz) δ 8.88 (app. dd, J=1.2, 5.1 Hz, 1H), 8.66 (d, J=7.8 Hz, 1H), app. dd, J=1.2, 8.1 Hz, 1H), 8.14 (d, J=6.9 Hz, 1H), 8.06 (t, J=7.8 Hz, 2H), 7.75 (t, J=8.1 Hz, 1H), 7.52 (dd, J=4.2, 8.7 Hz, 1H); MS (positive ion): Found 293 (MH⁺).

### EXAMPLE 24

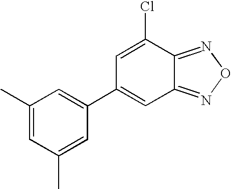

### 4-Chloro-6-[3,5-(dimethyl)phenyl]-2,1,3-benzoxadiazole (Cpd No. 322) and

### EXAMPLE 25

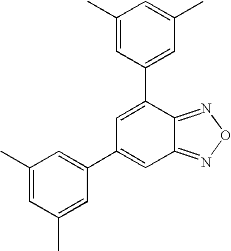

### 4,6-[3,5-(Dimethoxy)phenyl]-2,1,3-benzoxadiazole (Cpd No. 323)

These compounds were prepared according to the procedure for the preparation of Cpd. No. 285 and were separated by reverse phase liquid chromatography.

4-chloro-6-[3,5-(dimethyl)phenyl]-2,1,3-benzoxadiazole: ¹H NMR (CDCl₃, 300 MHz) δ 7.78 (d, J=1.5 Hz, 1H), 7.57 (s, 1H), 7.56 (s, 1H), 7.47 (d, J=1.8 Hz, 1H), 7.13 (br s, 1H), 2.43 (s, 6H); MS (positive ion): Found 259 (MH⁺).

4,6-bis[3,5-(dimethyl)phenyl]-2,1,3-benzoxadiazole: ¹H NMR (CDCl₃, 300 MHz) δ 7.85 (d, J=1.2 Hz, 1H), 7.78 (d, J=1.5 Hz, 1H), 7.61 (br s, 2H), 7.30 (br s, 2H), 7.11 (br s, 2H), 2.45 (s, 6H), 2.44 (s, 6H); MS (positive ion): Found 329 (MH⁺).

### EXAMPLE 26

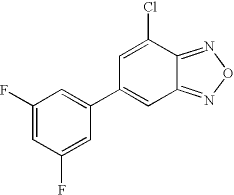

### 4-Chloro-6-[3,5-(difluoro)phenyl]-2,1,3-benzoxadiazole (Cpd No. 324)

This compound was prepared according to the procedure for the preparation of Cpd. No. 285 and was purified by reverse phase liquid chromatography.

¹H NMR (DMSO-d₆, 300 MHz) δ 8.39 (t, J=1.5 Hz, 1H), 8.05 (t, J=1.2 Hz, 1H), 7.88-7.84 (m, 2H), 7.45 (dtt, J=0.9, 2.1, 8.4 Hz, 1H); MS (positive ion): Found 267 (MH⁺).

### EXAMPLE 27

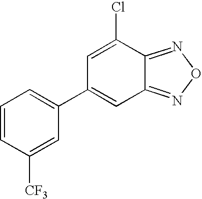

### 4-Chloro-6-[3-(trifluoromethyl)phenyl]-2,1,3-benzoxadiazole (Cpd No. 325)

This compound was prepared according to the procedure for the preparation of Cpd. No. 285 and was purified by reverse phase liquid chromatography.

¹H NMR (DMSO-d₆, 300 MHz) δ 8.40 (br s, 1H), 8.38-8.35 (m, 2H), 8.05 (d, J=1.2 Hz, 1H), 7.90-7.87 (m, 1H), 7.82-7.78 (m, 1H); MS (positive ion): Found 299 (MH⁺).

### EXAMPLE 28

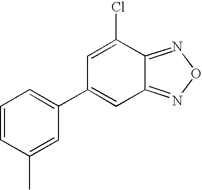

### 4-Chloro-6-(3-methylphenyl)-2,1,3-benzoxadiazole (Cpd No. 326)

This compound was prepared according to the procedure for the preparation of Cpd. No. 285 and was purified by reverse phase liquid chromatography.

¹H NMR (DMSO-d₆, 300 MHz) δ 8.27 (t, J=1.8 Hz, 1H), 7.86 (br s, 1H), 7.83-7.82 (m, 2H), 7.44 (t, J=7.8 Hz, 1H), 7.33 (d, J=7.8 Hz, 1H), 2.41 (s, 3H); MS (positive ion): Found 245 (MH⁺).

### EXAMPLE 29

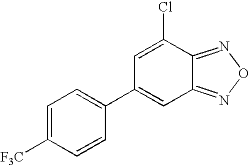

### 4-Chloro-6-[4-(trifluoromethyl)phenyl]-2,1,3-benzoxadiazole (Cpd No. 327)

This compound was prepared according to the procedure for the preparation of Cpd. No. 285 and was purified by reverse phase liquid chromatography.

¹H NMR (DMSO-d₆, 300 MHz) δ 8.39-8.38 (m, 1H), 8.28 (d, J=8.1 Hz, 2H), 8.01-8.00 (m, 1H), 7.93 (d, J=8.7 Hz, 2H); MS (positive ion): Found 299 (MH⁺).

### EXAMPLE 30

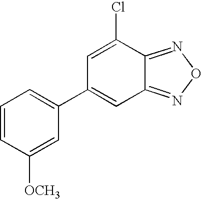

### 4-Chloro-6-(3-methoxyphenyl)-2,1,3-benzoxadiazole (Cpd No. 328)

This compound was prepared according to the procedure for the preparation of Cpd. 285 and was purified by reverse phase liquid chromatography.

¹H NMR (DMSO-d₆, 300 MHz) δ 8.29 (m, 1H), 7.89 (m, 1H), 7.65-7.62 (m, 1H), 7.59 (t, J=2.4 Hz, 1H), 7.47 (t, J=7.8 Hz, 1H), 7.09 (dd, J=2.7, 8.1 Hz, 1H), 3.84 (s, 3H); MS (positive ion): Found 261 (MH⁺).

### EXAMPLE 31

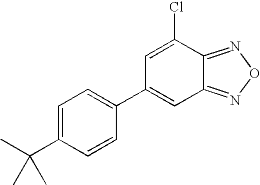

### 4-Chloro-6-(4-(tert-butyl)phenyl)-2,1,3-benzoxadiazole (Cpd No. 329)

This compound was prepared according to the procedure for the preparation of Cpd. No. 285 and was purified by reverse phase liquid chromatography.

¹H NMR (DMSO-d₆, 300 MHz) δ 8.24 (d, J=1.5 Hz, 1H), 7.98 (d, J=8.1 Hz, 2H), 7.80 (d, J=1.5 Hz, 1H), 7.56 (d, J=8.7 Hz, 2H), 1.33 (s, 9H); MS (positive ion): Found 287 (MH⁺).

### EXAMPLE 32

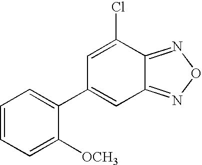

### 4-Chloro-6-(2-methoxyphenyl)-2,1,3-benzoxadiazole (Cpd No. 330)

This compound was prepared according to the procedure for the preparation of Cpd. No. 285 and was purified by reverse phase liquid chromatography.

¹H NMR (DMSO-d₆, 300 MHz) δ 8.28-8.27 (m, 1H), 7.60-7.59 (m, 1H), 7.57-7.55 (m, 1H), 7.52-7.46 (m, 1H), 7.21 (d, J=8.7 Hz, 1H), 7.09 (t, J=7.5 Hz, 1H), 3.76 (s, 3H); MS (negative ion): Found 259 (M−H⁺).

### EXAMPLE 33

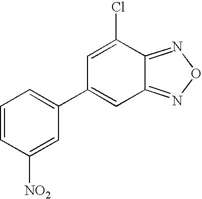

### 4-Chloro-6-(3-nitrophenyl)-2,1,3-benzoxadiazole (Cpd No. 331)

This compound was prepared according to the procedure for the preparation of Cpd. No. 285 and was purified by reverse phase liquid chromatography.

¹H NMR (DMSO-d₆, 300 MHz) δ 8.92 (t, J=2.1 Hz, 1H), 8.53-8.50 (m, 1H), 8.41-8.40 (m, 1H), 8.38-8.35 (m, 1H), 8.12-8.11 (m, 1H), 7.87 (t, J=8.1 Hz, 1H); MS positive ion): Found 276 (MH⁺).

### EXAMPLE 34

### Analysis of Compounds by LC/MS

The compounds of the invention were characterized by LC/MS using methods with various conditions. All the methods comprised a mobile phase that included 0.05% formic acid in water (component A) and 0.05% formic acid in acetonitrile (component B) for making the gradient. Table 1b below list the various conditions of the methods.

TABLE 1b

| Method | | | | |
|---|---|---|---|---|
| Name | Column type | Gradient | Temperature | Flow Rate |
| 7 min | Agilent SB-C18 2.1 mm × 50 mm, | 5-95% B over 5 | ambient | 0.8 mL/min. |
| | 5 μm | minutes, hold at 95% | | |
| | | B for 1 minute | | |
| 20 min_std | Agilent Zorbax SB-C18 | 5-100% B over 15 | ambient | 0.3 mL/min. |
| | Narrow-Bore 2.1 mm × 150 mm, | minutes, hold at 100% | | |
| | 5 μm | B for 3 minutes | | |
| NE_059510_{—} | Peeke Scientific Ultro | 5-95% B over 10 | ambient | 1.5 mL/min. |
| 13 | 120 C18Q 4.6 mm × 100 mm, | minutes | | |
| | 5 μm | | | |
| NE_0510010_{—} | Peeke Scientific Ultro | 5-100% B over 10 | ambient | 1.5 mL/min. |
| 13 | 120 C18Q 4.6 mm × 100 mm, | minutes, hold at 100% | | |
| | 5 μm | B for 2 minutes | | |
| NE_509010_{—} | Peeke Scientific Ultro | 50-90% B over 10 | ambient | 1.5 mL/min. |
| 15 | 120 C18Q 4.6 mm × 100 mm, | minutes, hold at 95% | | |
| | 5 μm | B for 2 minutes | | |
| NE_010010hold_{—} | Peeke Scientific Ultro | 5-100% B over 8 | ambient | 1.5 mL/min. |
| 15 | 120 C18Q 4.6 mm × 100 mm, | minutes, hold at 100% | | |
| | 5 μm | B for 4 minutes | | |
| 507515_20 | Peeke Scientific Ultro | 50-75% B over 15 | ambient | 1.5 mL/min. |
| | 120 C18Q 4.6 mm × 100 mm, | minutes, hold at 95% | | |
| | 5 μm | B for 2 minutes | | |
| 059515_20 | Peeke Scientific Ultro | 5-95% B over 15 | ambient | 1.5 mL/min. |
| | 120 C18Q 4.6 mm × 100 mm, | minutes, hold at 95% | | |
| | 5 μm | B for 2 minutes | | |
| 9 min | Agilent SB-C18 2.1 mm × 50 mm, | 5-95% B over 6 | ambient | 0.8 mL/min. |
| | 5 μm | minutes, hold at 95% | | |
| | | B for 2 minutes | | |
| 20 min | Agilent Zorbax SB-C18 | 5-100% B over 15 min, | ambient | 0.3 mL/min. |
| | Narrow-Bore 2.1 mm × 150 mm, | hold at 100% B | | |
| | 5 μm | for 3 minutes | | |
| 20 min_fast | Agilent Zorbax SB-C18 | 5-100% B over 10 | ambient | 0.3 mL/min. |
| | Narrow-Bore 2.1 mm × 150 mm, | minutes, hold at 100% | | |
| | 5 μm | B for 8 minutes | | |
| 509515_20 | Peeke Scientific Ultro | 50-95% B over 10 | ambient | 1.5 mL/min. |
| | 120 C18Q 4.6 mm × 100 mm, | minutes, hold at 95% | | |
| | 5 μm | B for 2 minutes | | |
| NE_0510010_{—} | Peeke Scientific Ultro | 5-100% B over 10 | ambient | 1.5 mL/min. |
| 15 | 120 C18Q 4.6 mm × 100 mm, | minutes, hold at 100% | | |
| | 5 μm | B for 2 minutes | | |
| 5010015_05 | Peeke Scientific Ultro | 50-100% B over 10 | ambient | 1.5 mL/min. |
| | 120 C18Q 4.6 mm × 100 mm, | minutes, hold at 95% | | |
| | 5 μm | B for 2 minutes | | |

Table 1c below shows the results of the LC/MS characterization of representative examples of the compounds of the invention.

TABLE 1c

| Cpd No. | Mol Weight (Tot) | Mass | Method Name | RT |
|---|---|---|---|---|
| 332 | 419.8451 | MH⁺ | 7 min | 4.16 |
| 334 | 529.6404 | MH⁺ | 7 min | 4.8 |
| 335 | 457.4877 | MH⁺ | 7 min | 4.05 |
| 336 | 313.7668 | MH⁺ | 7 min | 4.52 |
| 337 | 419.8451 | MH⁺ | 7 min | 3.89 |
| 338 | 446.4493 | MH⁺ | 7 min | 3.13 |
| 340 | 670.6327 | MH⁺ | 7 min | 5.43 |
| 341 | 404.23 | MH⁺ | 7 min | 4.13 |
| 345 | 327.7231 | MH⁺ | 7 min | 3.72 |
| 346 | 402.3812 | MH⁺ | 7 min | 3.89 |
| 347 | 351.8133 | MH⁺ | 7 min | 4.26 |
| 348 | 387.7757 | MH⁺ | 7 min | 4.37 |
| 350 | 391.7578 | M⁻¹ | 7 min | 4.26 |
| 351 | 341.75 | MH⁺ | 7 min | 3.97 |
| 352 | 365.8402 | MH⁺ | 7 min | 4.39 |
| 353 | 337.7864 | MH⁺ | 7 min | 4.13 |
| 354 | 358.2043 | MH⁺ | 7 min | 4.28 |
| 355 | 351.8133 | MH⁺ | 7 min | 4.24 |
| 356 | 339.7589 | MH⁺ | 7 min | 3.86 |
| 357 | 363.704 | MH⁺ | 7 min | 3.8 |
| 358 | 341.75 | MH⁺ | 7 min | 3.95 |
| 359 | 369.7852 | MH⁺ | 7 min | 3.92 |
| 359 | 369.7852 | MH⁺ | 7 min | 3.92 |
| 360 | 402.2141 | M⁻¹ | 20 m_std | 16.02 |
| 361 | 551.3632 | MH⁺ | 20 m_std | 18.3 |
| 362 | 388.2306 | M⁻¹ | 7 min | 4.37 |
| 363 | 348.7693 | MH⁺ | 7 min | 3.64 |
| 363 | 348.7693 | MH⁺ | 7 min | 3.59 |
| 364 | 345.7136 | MH⁺ | 7 min | 3.83 |
| 364 | 345.7136 | MH⁺ | 7 min | 3.83 |
| 365 | 378.6222 | M⁻¹ | 7 min | 4.22 |
| 366 | 393.7303 | MH⁺ | 7 min | 4.16 |
| 367 | 456.4792 | MH⁺ | 7 min | 4.24 |
| 368 | 314.7088 | MH⁺ | 7 min | 3.29 |
| 369 | 420.4462 | MH⁺ | 7 min | 3.8 |
| 370 | 362.7962 | MH⁺ | 7 min | 3.77 |
| 371 | 438.3622 | M⁻¹ | 7 min | 4.1 |
| 372 | 378.3841 | MH⁺ | 7 min | 3.57 |
| 373 | 340.2183 | MH⁺ | 7 min | 4.09 |
| 374 | 311.7082 | MH⁺ | 7 min | 2.222 |
| 375 | 220.231 | MH⁺ | 7 min | 2.49 |
| 376 | 470.4802 | MH⁺ | 7 min | 4.2 |
| 377 | 421.4082 | MH⁺ | 7 min | 3.31 |
| 378 | 449.4588 | MH⁺ | 7 min | 4.49 |
| 379 | 469.4924 | M⁻¹ | 7 min | 4.91 |
| 380 | 416.8445 | MH⁺ | 7 min | 3.86 |
| 381 | 353.7858 | MH⁺ | 7 min | 4.12 |
| 382 | 467.5054 | MH⁺ | 7 min | 3.74 |
| 383 | 375.7919 | MH⁺ | 7 min | 3.91 |
| 384 | 478.5525 | MH⁺ | 7 min | 15.12 |
| 385 | 426.4529 | MH⁺ | 7 min | 3.6 |
| 386 | 404.4468 | MH⁺ | 7 min | 3.89 |
| 387 | 374.8072 | MH⁺ | 7 min | 2.99 |
| 388 | 404.7901 | MH⁺ | 7 min | 3.11 |
| 389 | 425.4682 | MH⁺ | 7 min | 10.49 |
| 390 | 499.5739 | MH⁺ | 7 min | 3.81 |
| 391 | 375.7919 | MH⁺ | 7 min | 3.83 |
| 392 | 359.7925 | MH⁺ | 7 min | 4.06 |
| 393 | 375.7919 | MH⁺ | 7 min | 4.05 |
| 394 | 592.3962 | MH⁺ | 7 min | 4.58 |
| 395 | 378.3841 | MH⁺ | 7 min | 3.65 |
| 396 | 262.2187 | MH⁺ | 7 min | 3.75 |
| 397 | 292.2016 | MH⁺ | 7 min | 3.82 |
| 398 | 266.2622 | MH⁺ | 7 min | 2.59 |
| 399 | 353.7424 | M⁻¹ | 7 min | 3.2 |
| 400 | 444.3699 | MH⁺ | 7 min | 5.09 |
| 401 | 274.2111 | MH⁺ | 7 min | 3.63 |
| 402 | 435.432 | MH⁺ | NE_509010_15 | 3.76 |
| 403 | 396.3531 | MH⁺ | NE_0510010_15 | 9.19 |
| 404 | 414.4171 | MH⁺ | NE_059510_13 | 8.11 |
| 405 | 241.1973 | MH⁺ | NE_059510_13 | 7.53 |
| 406 | 241.1973 | MH⁺ | NE_059510_13 | 7.31 |
| 407 | 484.5592 | MH⁺ | 7 min | 4.39 |
| 408 | 486.5054 | MH⁺ | 7 min | 4.28 |
| 409 | 369.7852 | MH⁺ | 7 min | 4.17 |
| 410 | 369.7852 | MH⁺ | 7 min | 14.13 |
| 411 | 338.1707 | MH⁺ | 7 min | 3.5 |
| 412 | 388.8316 | MH⁺ | 7 min | 3.22 |
| 413 | 493.5641 | MH⁺ | 7 min | 4.93 |
| 414 | 380.1583 | MH⁺ | 7 min | 4.97 |
| 415 | 500.9648 | MH⁺ | 7 min | 4.91 |
| 416 | 394.2373 | MH⁺ | 7 min | 4.11 |
| 417 | 500.9648 | MH⁺ | 7 min | 4.55 |
| 418 | 408.3027 | M⁻¹ | 5010015_05 | 15.48 |
| 419 | 300.3599 | MH⁺ | 509515_20 | 14.02 |
| 420 | 408.3027 | MH⁺ | 509515_20 | 13.88 |
| 421 | 332.3587 | MH⁺ | 509515_20 | 11.25 |
| 422 | 384.5212 | MH⁺ | 20 m_fast | 17.17 |
| 423 | 332.3587 | MH⁺ | 20 min | 16.71 |
| 424 | 388.6287 | M⁻¹ | 20 m_std | 14.03 |
| 426 | 239.2768 | MH⁺ | 7 min | 4.43 |
| 427 | 320.212 | M⁻¹ | 9 min | 4.35 |
| 428 | 360.3201 | MH⁺ | 509515_20 | 7.75 |
| 429 | 392.3375 | MH⁺ | 509515_20 | 8.45 |
| 430 | 220.6147 | MH⁺ | 7 min | 3.68 |
| 431 | 392.3375 | MH⁺ | 7 min | 4.67 |
| 432 | 338.3661 | MH⁺ | 059515_20 | 13.37 |
| 433 | 380.4467 | MH⁺ | 059515_20 | 15.17 |
| 434 | 354.3655 | MH⁺ | 059515_20 | 12.75 |
| 435 | 267.3306 | MH⁺ | 7 min | 4.87 |
| 436 | 281.3141 | MH⁺ | 507515_20 | 8.22 |
| 437 | 259.1965 | MH⁺ | 7 min | 3.98 |
| 439 | 255.2329 | MH⁺ | 7 min | 4.15 |
| 440 | 241.206 | MH⁺ | 7 min | 4.03 |
| 441 | 283.2433 | MH⁺ | 7 min | 3.74 |
| 442 | 259.1965 | MH⁺ | 7 min | 4.08 |
| 443 | 283.2433 | MH⁺ | 7 min | 3.74 |
| 444 | 286.2036 | M⁻¹ | 7 min | 4.07 |
| 445 | 351.8133 | MH⁺ | 7 min | 4.26 |
| 446 | 324.7473 | MH⁺ | 7 min | 2.38 |
| 447 | 329.8072 | MH⁺ | 7 min | 4.39 |
| 450 | 254.2884 | MH⁺ | NE_059510_15 | 9.01 |
| 451 | 245.2378 | M⁻¹ | 7 min | 4.49 |
| 452 | 323.3718 | MH⁺ | 7 min | 3.71 |
| 453 | 309.3679 | MH⁺ | NE_059510_13 | 9.47 |
| 454 | 318.3752 | MH⁺ | 7 min | 5 |
| 455 | 324.7473 | MH⁺ | 7 min | 2.13 |
| 456 | 358.8054 | MH⁺ | 7 min | 2.88 |
| 457 | 258.3192 | MH⁺ | 7 min | 4.84 |
| 458 | 230.2655 | MH⁺ | 7 min | 4.35 |
| 459 | 258.3192 | MH⁺ | 7 min | 4.97 |
| 460 | 274.2753 | MH⁺ | 7 min | 4.28 |
| 461 | 258.3192 | MH⁺ | 7 min | 5.03 |
| 462 | 260.2917 | MH⁺ | 7 min | 4.37 |
| 463 | 290.318 | MH⁺ | 7 min | 4.02 |
| 464 | 333.3032 | MH⁺ | 7 min | 4.8 |
| 465 | 301.2586 | MH⁺ | 7 min | 3.82 |
| 467 | 285.2158 | MH⁺ | 7 min | 3.98 |
| 468 | 255.2329 | MH⁺ | 7 min | 4.32 |
| 469 | 291.2659 | MH⁺ | 7 min | 4.31 |
| 470 | 269.2598 | MH⁺ | 7 min | 4.34 |
| 471 | 269.2598 | MH⁺ | 7 min | 4.39 |
| 472 | 269.2598 | MH⁺ | 7 min | 4.53 |
| 473 | 292.2604 | MH⁺ | NE_059510_13 | 9.93 |
| 474 | 264.2067 | MH⁺ | NE_059510_13 | 9.49 |
| 475 | 308.2165 | MH⁺ | NE_059510_13 | 9.34 |
| 476 | 255.2329 | M⁻¹ | NE_010010_15 | 8.93 |
| 479 | 277.1869 | MH⁺ | NE_010010_15 | 8.48 |
| 482 | 271.2323 | MH⁺ | NE_010010_15 | 8.26 |
| 483 | 292.2537 | MH⁺ | NE_010010_15 | 7.62 |

RT is the retention time in minutes.

### Biological Examples

For the assays described below, the TRAF6/Uev1A/UBC13 assay is the biochemical plate-based assay of TRAF6 ligase activity using Ubc13 as the E2 enzyme. The gel-based assay is the biochemical solution-based TRAF6 ligase assay (by SDS-PAGE and Western blot) to confirm the plate-based ELISA assay. The APC assay is an assay which is another E3 ligase (APC2/APC11) biochemical assay.

### Biological Example 1

### TRAF6/Uev1A/Ubc13 Assay

A 96-well Ni-plate was blocked with 100 μl of 1% casein in PBS for 1 hour at room temperature. The plates were then washed three times with 200 μl of 1×PBS and 80 μl of a reaction buffer was added per well which contained 50 ng of Flag-ubiquitin, 50 ng of His-ubiquitin, 62.5 mM Tris pH 7.5, 6.25 mM MgCl₂, 1.0 mM DTT, and 2 μM ATP. To each well was added 10 μl of a solution of the compound in DMSO. The reaction was started by adding 10 μl of a solution consisting of 10 ng human E1, 25 ng each of Uev1a and Ubc13, and 100 ng TRAF6 in the reaction buffer. The plates were shaken for 10 minutes and incubate at room temperature for 1 hour. After the incubation, the plates were washed three times with 200 μl 1×PBS in 0.05% Tween and 100 μl of an antibody mix consisting of anti-Flag (1:30,000 dilution; Sigma F-3165) and anti-Mouse IgG-HRP (1/150,000 dilution; Jackson Immunoresearch #115-035-146) in 1×PBS with 0.25% BSA. The plates were then incubated for another 1 hour at room temperature, and after incubation the plate was washed three times with 200 μl 1×PBS with 0.05% Tween. The amount of ubiquitin was measured by adding 100 μl of Lumino substrate (1:5 dilution) and reading the luminescence with a fluorimeter. Table 2 contains data for this assay when example compounds of the invention were tested.

Biological Example 2

### ICAM Assay

The expression of intercellular adhesion molecule (ICAM)-1 in endothelial cells is pivotal in supporting lymphocyte migration across the vascular endothelium. TNF/IL-1 induces the NF-kB pathway in which NF-kB acts as a transcription factor and activation of NF-kB induces ICAM expression. IkB inhibits NF-KB by binding to it and retaining NF-kB in the cytoplasm. Upon cytokine stimulation, several signaling molecule including TRAF6, IKKs, and TAK1 are activated which lead to the phosphorylation of IkB. Phosphorylated IkB is then ubiquitinated by the SCF complex and degraded by proteasome, thereby releasing NF-kB to translocate into the nucleus. In the nucleus, NF-kB binds to DNA and activates transcription of various genes that are involved in inflammation, cell survival, and apoptosis. The ICAM assay is a primary assay for HTS as well as a cell based assay for TRAF6 inhibitors.

The following materials are used 96-well plates; F12K complete medium that includes 10% FBS and 1% P/S (penicillin/streptomycin); 1×PBS; 5 ug/ml IL-1, Human CD54 (CALTAG Laboratory, Cat#MHCD5400-4); anti-mouse-IgG (Jackson Lab, Cat#115-035 146); and A549 cells.

Procedure. 10,000 A549 cells/well were seeded in 100 μl F12K complete medium in a 96-well white plate. The seeded plate was incubate at 37° C. incubator in 5% CO₂ overnight. After the overnight incubation, 4 μl of diluted test compound was added and incubated for 1 hour at 37° C. After the 1 hour incubation, 4 μl of 25 ng/ml IL-1 was added to each well and the cells were stimulated at 37° C. for 4 hours.

After stimulation, the cells were stained with CD54 (1:1000 diluted with medium) and anti-mouse IgG (1:1100 diluted with medium) for 1 hour. Subsequently, the medium was removed and each well was washed with 200 μl of PBS three times. Detection of activity was performed by addition of 100 μl/well of lumino substrate.

Table 2 contains data for this assay when example compounds of the invention were tested.

### Biological Example 3

### Gel-Based E3 Ligase Assay

E3 (TRAF6) auto-ubiquitination was measured as described below. Activity in the presence of compound was determined relative to a parallel control in which only DMSO is added. The IC₅₀ values were typically determined using 6 or 8 different concentrations of compound, although as few as 2 concentrations may be used to approximate the IC₅₀ values.

E&K 96_well plates (E&K-20201) were used for the solution based biochemical assay. 80 μl of the reaction buffer were added to each well that contained 100 ng/well of Flag_ubiquitin. To this, 10 μl of the test compound diluted in DMSO were added. After the test compound was added, 10 μl of E1 (human), E2 (Ubc13/Uev1A) and TRAF6 in Protein Buffer were added to obtain a final concentration of 10 ng/well of E1, 25 ng/well of E2 and 100 ng/well TRAF6. The plates were shaken for 10 minutes and incubated at room temperature for 1 hour. After incubation, the reaction was stopped by adding 3311 of 4× loading buffer (non-reducing) per well and the plates were heated at 95° C. for 5 minutes. An aliquot of each well was run on a 4-12% Bis-Tris NuPage Gel and analyzed by Western Blot using anti-Flag as primary antibody, and HRP— conjugated anti-Mouse IgG as secondary antibody.

The Blocking Buffer contained 1% Casein in PBS. It was stored at 4° C. until used.

The reaction buffer consisted of 62.5 mM Tris pH 7.6 (Trizma Base—Sigma T-8524), 3 mM MgCl₂ (Magnesium Chloride—Sigma M-2393), 1 mM DTT (Sigma D-9779), 2.5 mM ATP (Roche Boehringer Mann Corp. 635-316), 100 ng/well of Flag-ubiquitin, 0.1% BSA (Sigma A-7906), and 0.05% Tween-20 (Sigma P-7949).

The Protein Buffer consisted of 20 mM Tris pH 7.6, 10% glycerol (Sigma G-5516) and 1 mM DTT.

The antibody mix consisted of 0.25% BSA (Sigma A-7906) in 1×PBS, 1/50,000 anti-Flag (Sigma F-3165), 1/100,000 of anti-Mouse IgG-HRP (Jackson Immunoresearch #115-035-146).

The substrate mix consisted of SuperSignal Substrate from Pierce (catalog number 37070ZZ) and was prepared by mixing 100 ml of the peroxide solution, 100 ml of the enhancer solution and 100 ml of Milli-Q water.

The data from these gel experiments was confirmatory and in agreement with the TRAF6/UB13 plate-based data.

### Biological Example 4

### APC-11/APC-2 Ligase Assay

E3 (His APC11/APC2— “APC”) auto ubiquitination was measured as described in U.S. patent application Ser. No. 09/826,312 (Publication No. U.S. 2002 0042083 A1), which is incorporated by reference in its entirety. Details of the protocol are described below. Activity in the presence of the compound was determined relative to a parallel control in which only DMSO was added. Values of the IC50 were typically determined using 6 or 8 different concentrations of the compound, although as few as 2 concentrations may be used to approximate the IC50 value.

Nickel coated 96 well plates (Pierce 15242) were blocked for 1 hour with 100 μl of blocking buffer at room temperature. The plates were washed 4 times with 225 μl of 1 □PBS and 80 μl of the reaction buffer were added that contained 100 ng/well of Flag ubiquitin. To this, 10 μl of the test compound diluted in DMSO were added. After the test compound was added, 10 μl of E1 (human), E2 (Ubch5c), and APC in Protein Buffer was added to obtain a final concentration of 5 ng/well of E1, 20 ng/well of E2 and 100 ng/well of APC. The plates were shaken for 10 minutes and incubated at room temperature for 1 hour. After incubation, the plates were washed 4 times with 225 μl of 1×PBS and 100 μl/well of Antibody Mix were added to each well. The plates were incubated at room temperature for another hour after which they were washed 4 times with 225 μl of 1×PBS and 100 μl/well of Lumino substrate were added to each well. The luminescence was measured by using a BMG luminescence microplate reader. The Blocking Buffer (1% Casein in 1×PBS) was stored at 4° C. until use.

The reaction buffer consisted of 62.5 mM Tris pH 7.6 (Trizma Base—Sigma T 8524), 3 mM MgCl2 (Magnesium Chloride—Sigma M 2393), 1 mM DTT (Sigma D 9779), 2.5 mM ATP (Roche Boehringer Mann Corp. 635 316), 100 ng/well of Flag ubiquitin, 0.1% BSA (Sigma A 7906), and 0.05% Tween 20 (Sigma P 7949).

The Protein Buffer consisted of 20 mM Tris pH 7.6, 10% glycerol (Sigma G 5516) and 1 mM DTT.

The antibody mix consisted of 0.25% BSA (Sigma A 7906) in 1×PBS, 1/50,000 anti Flag (Sigma F 3165), 1/100,000 of anti Mouse IgG HRP (Jackson Imrunoresearch #115 035 146).

The substrate mix consisted of SuperSignal Substrate from Pierce (catalog number 37070ZZ) and was prepared by mixing 100 ml of the peroxide solution, 100 ml of the enhancer solution and 100 ml of Milli Q® water.

### Biological Example 5

### ROC1/CUL1 Ubiquitin Ligase Assay (SCF Assay)

Inhibition of ubiquitin ligase activity of E1+E2+E3 was measured using the protocol as described in WO 01/75145 with E3 as the ROC1/CUL1, ROC1/CUL2, or ROC2/CUL5 complex.

Materials and Methods

The wells of nickel-substrate 96-well plates (Pierce Chemical) are blocked with 100 μl of 1 casein/phosphate buffered saline (PBS) for 1 hour at room temperature, then washed with 200 μl of PBST (0.1% Tween-20 in PBS) 3 times. To each well is added the following Flag-ubiquitin (see above) reaction solution: 62.5 mM Tris pH 7.5, 6.25 mm MgCl2, 0.75 mM DTT, 2.5 mM ATP, 2.5 mM NaF1, 2.5 nM Okadaic acid, 100 ng Flag-ubiquitin (made as described above).

The buffer solution is brought to a final volume of 80 μl with Milipore-filtered water, followed by the addition of 10 μl DMSO.

To the above solution is then added 10 μl of ubiquitination enzymes in 20 mM Tris buffer, pH 7.5, and 5% glycerol. E2-Ubch5c and E3-His ROC1/Cul1, ROC1/CUL2, and ROC2/CUL5 are made as described in WO 01/75145. E1 is obtained commercially (Affiniti Research Products, Exeter, U. K.). The following amounts of each enzyme are used for these assays: 5 ng/well of E1; 25 nl/well E2; and 100 ng/well His-E3. Varying amounts of compounds according to the invention are added and the reaction allowed to proceed at room temperature for 1 hour.

Following the ubiquitination reaction, the wells are washed with 200 μl of PBST 3 times. For measurement of the enzyme-bound ubiquitin, 100 gel of Mouse anti-Flag (1:10,000) and anti-Mouse Ig-HRP (1:15, 000) in PBST are added to each well and allowed to incubate at room temperature for 1 hour. The wells are then washed with 200 μl of PBST 3 times, followed by the addition of 100 μl of luminol substrate (1/5 dilution). Luminescence for each well is then measured using a fluorimeter.

Table 2 below illustrates the inhibitory activity of the compounds of the invention determined by the TRAF6/Uev1A/Ubc13 assay as well as in the ICAM, APC and the ROC1/CUL1 Ubiquitin Ligase Assay (SCF Assay). The IC₅₀ values were determined using a various concentrations of the compounds.

TABLE 2

| | TRAF6/ | | | |
|---|---|---|---|---|
| | Ubc13 | | APC | |
| Cpd No. | IC₅₀ | ICAM | IC₅₀ | SCF |
| 1 | + | | | |
| 5 | ++ | | ++ | |
| 6 | + | | | |
| 8 | + | | | |
| 9 | + | | | |
| 10 | + | | | |
| 11 | ++ | | | |
| 12 | + | | | |
| 13 | + | | | |
| 62 | + | | | |
| 72 | + | | | |
| 73 | ++ | | ++ | |
| 78 | ++ | | ++ | |
| 79 | ++ | | | |
| 80 | + | | | |
| 81 | + | | | |
| 82 | + | | | |
| 84 | ++ | | ++ | |
| 85 | + | | | |
| 86 | + | | | |
| 87 | + | | | |
| 88 | + | | | |
| 90 | ++ | | ++ | |
| 91 | + | | | |
| 92 | + | | | |
| 93 | ++ | | | |
| 94 | ++ | | ++ | |
| 101 | + | | | |
| 102 | + | | | |
| 103 | + | | | |
| 104 | + | | | |
| 105 | + | | | |
| 106 | + | | | |
| 107 | + | | | |
| 108 | + | | | |
| 109 | ++ | | ++ | |
| 110 | + | | | |
| 112 | + | | | |
| 113 | ++ | | ++ | |
| 114 | ++ | | ++ | |
| 115 | + | | | |
| 116 | + | | | |
| 117 | + | | | |
| 118 | ++ | | | |
| 121 | ++ | | ++ | |
| 122 | ++ | | ++ | |
| 133 | + | | | + |
| 134 | + | | | + |
| 135 | + | | | + |
| 136 | + | | | + |
| 137 | + | | | + |
| 139 | ++ | | | |
| 140 | ++ | | ++ | |
| 141 | ++ | | ++ | |
| 142 | ++ | | ++ | |
| 143 | + | | | |
| 144 | ++ | | ++ | |
| 145 | ++ | | ++ | |
| 146 | ++ | | ++ | |
| 147 | ++ | | | |
| 148 | ++ | | ++ | |
| 149 | ++ | + | ++ | |
| 150 | + | + | | |
| 151 | ++ | | | |
| 152 | + | | | |
| 153 | ++ | | ++ | |
| 154 | + | | | |
| 155 | ++ | | | |
| 156 | + | | | |
| 157 | ++ | + | ++ | |
| 158 | ++ | + | ++ | |
| 159 | ++ | | ++ | |
| 160 | + | | | |
| 161 | + | | | |
| 162 | + | | | |
| 163 | ++ | | | |
| 164 | +++ | | | |
| 165 | + | | | |
| 166 | ++ | | | |
| 167 | + | + | | |
| 168 | + | | | |
| 169 | ++ | | | |
| 170 | ++ | + | | |
| 171 | ++ | | ++ | |
| 172 | + | | | |
| 173 | + | | | |
| 174 | + | | | |
| 175 | +++ | | | |
| 176 | ++ | | | |
| 177 | ++ | | ++ | |
| 178 | + | + | | |
| 179 | ++ | + | ++ | |
| 180 | + | + | | |
| 181 | ++ | | ++ | |
| 182 | + | + | | |
| 183 | ++ | | ++ | |
| 184 | + | + | | |
| 185 | + | + | | |
| 186 | + | + | | |
| 187 | ++ | | | |
| 188 | + | + | | |
| 189 | + | + | | |
| 190 | ++ | | ++ | |
| 191 | + | | | |
| 192 | + | + | | + |
| 193 | ++ | | ++ | ++ |
| 194 | + | | | |
| 195 | + | | | |
| 196 | ++ | | | + |
| 197 | ++ | | | + |
| 198 | + | | | |
| 199 | + | | | + |
| 200 | + | + | | |
| 201 | + | + | | |
| 202 | + | + | | |
| 203 | ++ | + | | + |
| 204 | + | | | |
| 205 | + | | | |
| 206 | ++ | | | + |
| 207 | + | + | | |
| 209 | ++ | + | | + |
| 210 | + | | | + |
| 211 | + | | | |
| 212 | ++ | | | + |
| 213 | + | | | |
| 214 | ++ | + | | + |
| 215 | ++ | + | | + |
| 216 | ++ | | | + |
| 217 | ++ | + | | |
| 218 | + | | | |
| 219 | ++ | | | |
| 220 | + | + | | |
| 221 | + | | | |
| 222 | ++ | | | + |
| 223 | ++ | | | + |
| 224 | + | | | |
| 225 | + | + | | |
| 226 | ++ | + | | + |
| 227 | + | | | |
| 228 | +++ | | | + |
| 229 | ++ | + | | |
| 230 | ++ | | | |
| 231 | ++ | | | |
| 232 | ++ | | | |
| 233 | +++ | | | |
| 234 | + | + | | |
| 235 | + | | | |
| 236 | + | + | | |
| 237 | + | | | |
| 238 | ++ | + | | + |
| 240 | + | | | |
| 242 | + | | | |
| 243 | + | | | |
| 245 | ++ | | | + |
| 246 | ++ | | | |
| 247 | + | | | |
| 248 | + | | | |
| 249 | ++ | | | |
| 250 | + | | | |
| 251 | + | | | |
| 252 | + | | | |
| 253 | ++ | | | |
| 254 | ++ | | | |
| 255 | ++ | | | + |
| 256 | + | | | + |
| 257 | + | | | + |
| 258 | ++ | + | | + |
| 259 | ++ | | | + |
| 260 | ++ | + | | + |
| 261 | ++ | | | + |
| 262 | + | + | | + |
| 263 | + | + | | + |
| 264 | + | | | + |
| 265 | + | | | |
| 266 | + | | | + |
| 267 | + | | | + |
| 268 | + | + | | + |
| 269 | ++ | + | | + |
| 270 | + | | | + |
| 271 | ++ | + | | |
| 272 | + | | | |
| 273 | + | | | |
| 274 | ++ | + | | |
| 275 | ++ | | | |
| 276 | ++ | | | |
| 277 | + | | | |
| 278 | + | | | |
| 279 | + | | | |
| 280 | + | | | |
| 281 | ++ | | | ++ |
| 282 | + | | | |
| 283 | + | | | |
| 284 | ++ | | | + |
| 285 | + | | | + |
| 286 | + | | ++ | |
| 287 | + | | | + |
| 288 | + | | | + |
| 289 | + | | | |
| 290 | + | | | |
| 291 | ++ | + | | |
| 292 | + | | | |
| 293 | + | | | |
| 294 | + | | | |
| 295 | + | | | |
| 296 | ++ | | | |
| 297 | + | | | |
| 298 | + | | | |
| 299 | + | | | |
| 300 | + | | | |
| 301 | + | | | |
| 302 | ++ | | | |
| 303 | + | | | |
| 304 | + | | | |
| 305 | + | | | |
| 306 | + | | | |
| 307 | + | | | |
| 308 | + | ++ | ++ | |
| 309 | ++ | ++ | ++ | |
| 310 | + | ++ | ++ | |
| 311 | ++ | ++ | ++ | |
| 312 | ++ | + | ++ | |
| 313 | ++ | ++ | ++ | |
| 314 | ++ | | ++ | |
| 315 | +++ | | +++ | |
| 332 | + | + | ++ | |
| 334 | + | ++ | + | |
| 335 | + | + | + | |
| 336 | + | + | + | |
| 337 | + | +++ | + | |
| 339 | + | + | + | |
| 340 | + | + | + | |
| 341 | + | ++ | + | |
| 342 | ++ | ++ | ++ | |
| 343 | + | ++ | + | |
| 344 | + | + | + | |
| 345 | + | ++ | + | |
| 346 | + | + | ++ | |
| 347 | + | ++ | + | |
| 348 | + | ++ | + | |
| 349 | + | + | ++ | |
| 350 | + | ++ | + | |
| 351 | + | ++ | + | |
| 352 | + | ++ | + | |
| 353 | + | ++ | + | |
| 354 | + | ++ | + | |
| 355 | + | + | + | |
| 356 | + | ++ | + | |
| 357 | + | + | + | |
| 358 | + | + | + | |
| 359 | + | ++ | + | |
| 360 | + | ++ | + | |
| 361 | ++ | + | + | |
| 362 | + | ++ | + | |
| 363 | + | ++ | + | |
| 364 | + | + | + | |
| 365 | + | + | + | |
| 366 | + | ++ | + | |
| 367 | + | + | + | |
| 368 | + | + | + | |
| 369 | + | + | + | |
| 370 | + | ++ | + | |
| 371 | + | + | + | |
| 372 | + | + | + | |
| 373 | + | ++ | ++ | |
| 374 | + | + | + | |
| 375 | + | + | + | |
| 376 | + | + | ++ | |
| 377 | + | + | + | |
| 378 | + | + | ++ | |
| 379 | ++ | + | ++ | |
| 380 | + | ++ | + | |
| 381 | + | ++ | + | |
| 382 | + | + | + | |
| 383 | ++ | ++ | ++ | |
| 384 | ++ | + | + | |
| 385 | + | + | + | |
| 386 | + | + | + | |
| 387 | + | ++ | + | |
| 388 | + | + | + | |
| 389 | + | + | + | |
| 390 | ++ | + | ++ | |
| 391 | ++ | ++ | ++ | |
| 392 | + | ++ | + | |
| 393 | ++ | ++ | ++ | |
| 394 | ++ | ++ | ++ | |
| 395 | + | + | + | |
| 396 | + | + | + | |
| 397 | + | + | + | |
| 398 | + | ++ | + | |
| 399 | + | + | + | |
| 401 | + | + | + | |
| 402 | + | + | ++ | |
| 403 | + | + | + | |
| 404 | + | + | + | |
| 405 | + | + | + | |
| 406 | + | + | + | |
| 407 | ++ | ++ | ++ | |
| 408 | + | ++ | + | |
| 409 | + | ++ | + | |
| 410 | + | ++ | + | |
| 411 | ++ | ++ | + | |
| 412 | + | ++ | + | |
| 413 | ++ | ++ | ++ | |
| 414 | ++ | ++ | ++ | |
| 415 | ++ | ++ | ++ | |
| 416 | ++ | ++ | ++ | |
| 417 | ++ | ++ | ++ | |
| 418 | + | + | + | |
| 419 | + | + | + | |
| 420 | + | + | + | |
| 421 | + | + | + | |
| 422 | + | + | + | |
| 423 | + | + | + | |
| 424 | + | + | + | |
| 425 | + | + | + | |
| 426 | + | + | + | |
| 427 | + | ++ | + | |
| 428 | + | + | + | |
| 429 | + | + | + | |
| 430 | + | + | + | |
| 431 | + | + | + | |
| 432 | + | + | + | |
| 433 | + | + | + | |
| 434 | + | + | + | |
| 435 | + | + | + | |
| 436 | + | + | + | |
| 437 | + | ++ | ++ | |
| 438 | + | + | + | |
| 439 | + | ++ | + | |
| 440 | + | + | ++ | |
| 441 | ++ | ++ | ++ | |
| 442 | ++ | ++ | + | |
| 443 | ++ | ++ | ++ | |
| 444 | ++ | ++ | ++ | |
| 445 | + | + | + | |
| 446 | + | ++ | + | |
| 447 | + | ++ | + | |
| 448 | + | ++ | + | |
| 449 | + | + | + | |
| 450 | + | + | + | |
| 451 | + | ++ | + | |
| 452 | + | + | + | |
| 453 | + | + | ++ | |
| 454 | + | + | ++ | |
| 455 | + | ++ | + | |
| 456 | + | + | + | |
| 457 | + | + | + | |
| 458 | + | + | + | |
| 459 | + | + | + | |
| 460 | + | + | + | |
| 461 | + | + | + | |
| 462 | + | + | + | |
| 463 | + | + | + | |
| 464 | ++ | ++ | ++ | |
| 465 | + | ++ | + | |
| 466 | ++ | ++ | + | |
| 467 | ++ | ++ | ++ | |
| 468 | + | ++ | ++ | |
| 469 | + | ++ | ++ | |
| 470 | + | ++ | ++ | |
| 471 | + | ++ | ++ | |
| 472 | ++ | ++ | ++ | |
| 473 | + | + | + | |
| 474 | + | + | + | |
| 475 | + | + | + | |
| 476 | + | + | + | |
| 477 | + | ++ | ++ | |
| 478 | + | ++ | ++ | |
| 479 | + | + | + | |
| 480 | + | ++ | ++ | |
| 481 | ++ | ++ | ++ | |
| 482 | + | + | + | |
| 483 | + | + | + | |
| 484 | + | + | + | |
| 485 | + | | | + |
| 486 | ++ | | ++ | ++ |

+++ means less than 1 μM
++ means between 1 and 20 μM
+ means 20 μM or greater

Table 3 illustrates the inhibitory activity of the compounds of the invention determined by the PAD assay described above in a number of cell types. Apoptosis was not

### Biological Example 6

### Cell Proliferation Assays

Cell Culture Preparation

Cell lines used are available from American Type Culture Collection (ATCC), for example, cell cultures containing A549 (ATCC# CCL-185), HeLa (ATCC# CCL-2), HCT116 ATTC# CCL-247), and H1299 (ATCC# CRL-5803) cells were maintained in T175 flasks following the ATCC recommended media and handling procedures. Flasks reaching approximately 70% confluency were trypsinized and resuspended in RPMI media (Cell-Gro catalog number 10-040-CM) modified to contain 5% FBS, 100 ug/mlPen/Strep (Cell-Gro catalog number 30-002-CL), and 0.3 mg/ml L-Glutamine (Cell-Gro catalog number 25-003-CL). A 20,000 cells/ml solution was made for plating. Cells were plated in black Packard 96 well plates by placing 100 μl per well (2,000 cells per well). Table 3 below shows these and additional cell line data. The definitions of the cell types used are as follows: A549 is lung carcinoma; HI 299 is non-small cell lung carcinoma; HI 155 is non-small cell lung carcinoma; AsPC-1 is pancreatic adenocarcinoma; Caov-3 is ovarian adenocarcinoma; COLO 205 is colorectal adenocarcinoma; DLD-1 colorectal adenocarcinoma; HCT116 is colorectal carcinoma; DU 145 is prostate carcinoma; ES-2 is ovarian clear cell carcinoma; H460 is large cell lung carcinoma; HELA is cervical adenocarcinoma; MIA PaCa-2 is pancreatice carcinoma; OVCAR-3 is ovarian adenocarcinoma; OVCAR8 is ovarian carcinoma; PC3 is prostate adenocarcinoma; SK-OV-3 is ovarian adenocarcinoma; SU86.86 is pancreatic carcinoma; TOV-21G is avarian clear cell carcinoma; U2OS is bone osteosarcoma; is ASPC-I is pancreatic adenocarcinoma; BXPC-3 is pabcreatic adenocarcinoma; HL60 is promyeloblast promyelocytic leukemia; K562 is bone marrow chronic myelogenous leukemia; L1210 is mouse lymphocytic leukemia; MOLT3 is T lymphoblast acute lymphoblastic leukemia; MOLT4 is T lymphoblast acute lymphoblastic leukemia; SW620 is colorectal adenocarcinoma; THP-1 is monocyte acute monocytic leukemia; U937 is histiocytic lymphoma; and UACC-257 is melanoma.

Cell Treatment with Compounds

Compounds and additional media were added 24 hours after cell plating. A compound master plate was created with concentrations 500 times greater than the final concentration added to the cells. All compound testing was done in duplicate using 6.3 fold dilutions starting with 10 mM. All outside wells (and 4 internal wells) were DMSO controls. Taxol and at least one additional control were run on all plates. Three microliters of the compound master plate were added to deep well blocks containing 750 μl of RPMI media. One hundred microliters were transferred from the compound/media deep well blocks to the plated cells resulting in a 500 fold dilution of the compounds. Cells were grown at 37° C., 5% CO₂ for 48 hours.

Photographic Image Analysis of Proliferation, Apoptosis and Death (PAD Assay)

Cells to be analyzed by photography were fixed and stained. One hundred microliters of media were removed and 100 μl of 9.3% formamide was added to each well. Plates were left on the benchtop for 45 minutes. A staining solution containing 1.55 μl of 1 mg/ml DAPI added to 18.75 ml PBS was warmed for 15 minutes at 37° C. The cells were aspirated prior to washing with 100 μl of PBS. Seventy microliters of PBS were aspirated and 170 μl of the DAPI solution were added to each well of fixed cells. Plates were left at room temperature for one hour then aspirated and washed twice with 100 μl of PBS. The stained cells were left at 4° C. for a minimum of 16 hours before photographic analysis with Array Scan II (Cellomics). Analysis of the photographic images to determine numbers of live cells (proliferation), apoptotic cells and dead cells, were according to the methods described in U.S. Utility patent application Ser. No. 10/652,440, which incorporated herein in its entirety.

Non-photographic Proliferation Analysis

Some cell plates were treated with Promega Cell titer Aqueous 1 kit (promega-VWR catalog number G3580). In this case, 48 hours after the test compound were added, 100 μl of media were removed and 20 μl of cell titer reagent were added to all wells. Plates were incubated at 37° C. for 45 minutes prior to absorbance reads on the Wallac plate reader at 490 nm for 0.1 sec/well. Results were similar to those obtained via the photographic analysis (described above).

TABLE 3

| | | A549 | DLD1 | H1299 | H1299 | HCT116 | HELA | OVCAR8 | U2OS | UACC257 | Colo205 | HCT116 | Miapaca2 |
|---|---|---|---|---|---|---|---|---|---|---|---|---|---|
| Cpd No. | A549 3pt | 6pt | 6pt | 3pt | 6pt | 6pt | 6pt | 6pt | 6pt | 6pt | 9pt | 9pt | 9pt |
| 5 | | +++ | | | +++ | | | +++ | | +++ | | | |
| 8 | + | | | | | | | | | | | | |
| 9 | + | ++ | | + | + | | | + | | + | | | |
| 10 | | + | | | + | | | | | | | | |
| 11 | + | | | + | | | | + | | + | | | |
| 12 | + | | | + | | | | | | | | | |
| 13 | + | | | + | | | | | | | | | |
| 14 | + | | | + | | | | | | | | | |
| 15 | + | | | ++ | | | | | | | | | |
| 16 | + | | | + | | | | | | | | | |
| 18 | + | | | + | | | | | | | | | |
| 19 | + | | | + | | | | | | | | | |
| 21 | + | | | ++ | | | | | | | | | |
| 22 | + | | | + | | | | | | | | | |
| 23 | + | | | + | | | | | | | | | |
| 24 | ++ | | | ++ | | | | | | | | | |
| 26 | + | | | + | | | | + | | + | | | |
| 27 | + | | | + | | | | | | | | | |
| 43 | ++ | | | + | | | | | | | | | |
| 46 | + | | | + | | | | | | | | | |
| 49 | + | | | + | | | | | | | | | |
| 50 | + | | | + | | | | | | | | | |
| 51 | + | | | + | | | | | | | | | |
| 52 | + | | | + | | | | | | | | | |
| 53 | + | | | + | | | | | | | | | |
| 54 | + | | | + | | | | | | | | | |
| 55 | + | | | + | | | | | | | | | |
| 56 | + | | | + | | | | | | | | | |
| 57 | + | | | + | | | | | | | | | |
| 58 | + | | | + | | | | | | | | | |
| 59 | + | | | + | | | | | | | | | |
| 60 | + | | | + | | | | | | | | | |
| 62 | + | | | + | | | | | | | | | |
| 63 | + | | | + | | | | | | | | | |
| 64 | ++ | +++ | +++ | ++ | +++ | ++ | ++ | | +++ | | | | |
| 65 | + | | | + | | | | | | | | | |
| 66 | + | | | + | | | | | | | | | |
| 67 | + | | | + | | | | | | | | | |
| 68 | + | | | + | | | | | | | | | |
| 71 | ++ | | | + | | | | | | | | | |
| 72 | + | | | + | | | | | | | | | |
| 73 | + | ++ | | + | ++ | | | + | | + | | | |
| 74 | + | | | + | | | | | | | | | |
| 76 | + | | | + | | | | | | | | | |
| 77 | + | | | + | | | | | | | | | |
| 78 | | | | | | | | ++ | | ++ | | | |
| 79 | ++ | ++ | | ++ | ++ | | | ++ | | + | | | |
| 80 | + | | | + | | | | | | | | | |
| 82 | + | | | + | | | | | | | | | |
| 85 | + | | | + | | | | | | | | | |
| 86 | + | | | + | | | | | | | | | |
| 87 | + | | | + | | | | | | | | | |
| 88 | + | | | + | | | | | | | | | |
| 90 | + | ++ | | + | ++ | | | + | | + | | | |
| 91 | + | | | + | | | | | | | | | |
| 92 | + | + | | + | + | | | + | | + | | | |
| 93 | | + | | | + | | | | | | | | |
| 94 | | | | | | | | ++ | | ++ | | | |
| 95 | + | | | + | | | | | | | | | |
| 96 | + | | | + | | | | | | | | | |
| 97 | + | | | + | | | | | | | | | |
| 98 | + | | | + | | | | | | | | | |
| 100 | + | | | + | | | | | | | | | |
| 101 | + | | | + | | | | | | | | | |
| 102 | + | | | + | | | | | | | | | |
| 103 | + | | | + | | | | | | | | | |
| 104 | + | | | + | | | | | | | | | |
| 105 | + | | | + | | | | | | | | | |
| 106 | + | | | + | | | | | | | | | |
| 107 | + | + | | + | + | | | + | | + | | | |
| 108 | ++ | | | + | | | | | | | | | |
| 109 | + | | | + | | | | | | | | | |
| 110 | + | | | + | | | | | | | | | |
| 112 | + | | | + | | | | | | | | | |
| 113 | ++ | | | ++ | | | | ++ | | + | | | |
| 114 | | | | | | | | ++ | | ++ | | | |
| 115 | + | | | + | | | | | | | | | |
| 116 | ++ | | | + | | | | | | | | | |
| 118 | + | | | + | | | | | | | | | |
| 119 | + | | | + | | | | | | | | | |
| 120 | + | | | + | | | | | | | | | |
| 121 | + | | | + | | | | | | | | | |
| 123 | + | | | + | | | | | | | | | |
| 124 | + | | | + | | | | | | | | | |
| 125 | + | | | + | | | | | | | | | |
| 126 | + | | | + | | | | | | | | | |
| 127 | + | | | + | | | | | | | | | |
| 128 | + | | | + | | | | | | | | | |
| 129 | + | | | + | | | | | | | | | |
| 130 | + | | | + | | | | | | | | | |
| 131 | + | | | + | | | | | | | | | |
| 132 | + | | | + | | | | | | | | | |
| 139 | + | | | + | | | | | | | | | |
| 140 | + | | | + | | | | ++ | | ++ | | | |
| 141 | + | | | + | | | | + | | + | | | |
| 142 | + | | | + | | | | ++ | | ++ | | | |
| 143 | + | | | + | | | | | | | | | |
| 144 | | | | + | | | | + | | + | | | |
| 145 | | | | + | | | | + | | + | | | |
| 146 | ++ | | | ++ | | | | ++ | | ++ | | | |
| 148 | + | | | + | | | | ++ | | ++ | | | |
| 149 | | ++ | | | +++ | | | ++ | | ++ | | | |
| 150 | +++ | +++ | +++ | ++ | +++ | +++ | +++ | | +++ | | | | |
| 151 | + | | | + | | | | | | | | | |
| 152 | ++ | | | ++ | | | | | | | | | |
| 153 | + | | | + | | | | + | | + | | | |
| 154 | + | | | + | | | | | | | | | |
| 155 | + | | | + | | | | | | | | | |
| 156 | + | | | + | | | | | | | | | |
| 157 | | ++ | | | +++ | | | ++ | | ++ | | | |
| 158 | | | | | | | | ++ | | ++ | | | |
| 159 | | | | | | | | ++ | | + | | | |
| 160 | + | | | + | | | | | | | | | |
| 161 | + | | | + | | | | | | | | | |
| 162 | + | | | + | | | | | | | | | |
| 163 | + | | | + | | | | | | | | | |
| 164 | ++ | ++ | | ++ | ++ | | | | + | | | | |
| 165 | ++ | + | | + | + | | | | | | | | |
| 166 | | ++ | | | + | | | | | | | | |
| 167 | ++ | + | | ++ | + | | | | +++ | | | | |
| 168 | ++ | + | | ++ | + | | | | | | | | |
| 169 | + | ++ | | + | +++ | | | | +++ | | | | |
| 170 | ++ | + | | ++ | + | | | | | | | | |
| 171 | + | + | | ++ | + | | | | +++ | | | | |
| 172 | ++ | + | | ++ | + | | | | | | | | |
| 173 | | ++ | | | ++ | | | | | | | | |
| 174 | ++ | ++ | | ++ | ++ | | | | | | | | |
| 175 | + | ++ | | + | ++ | | | | | | | | |
| 176 | + | + | | + | + | | | | | | | | |
| 177 | + | + | | + | + | | | | | | | | |
| 178 | ++ | + | +++ | ++ | +++ | +++ | +++ | | +++ | | | | |
| 179 | ++ | ++ | ++ | ++ | +++ | ++ | ++ | | +++ | | | | |
| 180 | ++ | + | | ++ | + | | | | | | | | |
| 181 | + | ++ | | + | + | | | | | | | | |
| 182 | | ++ | | | ++ | | | | | | | | |
| 184 | ++ | + | | ++ | + | | | | | | | | |
| 185 | | ++ | | | ++ | | | | | | | | |
| 186 | ++ | ++ | | ++ | +++ | | | | | | | | |
| 187 | + | | | + | | | | | | | | | |
| 188 | ++ | + | | ++ | +++ | | | | +++ | | | | |
| 189 | ++ | | | ++ | | | | | | | | | |
| 191 | ++ | | | ++ | | | | | | | | | |
| 192 | ++ | + | | ++ | + | | | | | | | | |
| 194 | + | + | | ++ | + | | | | +++ | | | | |
| 195 | + | | | + | | | | | | | | | |
| 196 | + | | | + | | | | | | | | | |
| 197 | ++ | | | ++ | | | | | | | | | |
| 199 | ++ | + | | ++ | + | | | | | | | | |
| 200 | ++ | + | | ++ | +++ | | | | | | | | |
| 201 | ++ | + | | ++ | +++ | | | | +++ | | | | |
| 202 | ++ | +++ | +++ | ++ | +++ | +++ | ++ | | +++ | | | | |
| 204 | ++ | + | +++ | +++ | +++ | ++ | ++ | | +++ | | | | |
| 205 | + | | | ++ | | | | | | | | | |
| 206 | ++ | | | + | | | | | | | | | |
| 207 | ++ | | | ++ | | | | | | | | | |
| 208 | ++ | | | ++ | | | | | | | | | |
| 209 | ++ | +++ | | ++ | +++ | | | | | | | | |
| 210 | + | | | ++ | | | | | | | | | |
| 211 | + | | | + | | | | | | | | | |
| 212 | ++ | | | ++ | | | | | | | | | |
| 213 | ++ | | | + | | | | | | | | | |
| 214 | ++ | | | ++ | | | | | | | | | |
| 217 | | ++ | | | ++ | | | | | | | | |
| 218 | + | + | ++ | ++ | ++ | ++ | ++ | | ++ | | | | |
| 219 | ++ | + | +++ | ++ | +++ | +++ | + | | +++ | | ++ | +++ | +++ |
| 220 | ++ | ++ | ++ | ++ | ++ | ++ | ++ | | ++ | | | | |
| 221 | + | | | + | | | | | | | | | |
| 222 | + | | | ++ | | | | | | | | | |
| 224 | ++ | | | + | | | | | | | | | |
| 225 | ++ | | | ++ | | | | | | | | | |
| 226 | | ++ | | | ++ | | | | | | | | |
| 227 | ++ | +++ | | +++ | +++ | | | | | | | | |
| 228 | + | | | + | | | | | | | | | |
| 229 | | ++ | | | +++ | | | | | | | | |
| 230 | ++ | | | ++ | | | | | | | | | |
| 231 | + | | | ++ | | | | | | | | | |
| 232 | ++ | | | ++ | | | | | | | | | |
| 233 | + | | | + | | | | | | | | | |
| 234 | ++ | | | ++ | | | | | | | | | |
| 235 | + | | | + | | | | | | | | | |
| 236 | ++ | | | ++ | | | | | | | | | |
| 242 | | + | | | + | | | | | | | | |
| 243 | | +++ | | | + | | | | | | | | |
| 245 | | + | | | +++ | | + | | | | +++ | +++ | +++ |
| 246 | | + | | | ++ | | | | | | | | |
| 257 | + | | | + | | | | | | | | | |
| 258 | +++ | | | ++ | | | | | | | | | |
| 259 | ++ | | | ++ | | | | | | | | | |
| 260 | ++ | | | ++ | | | | | | | | | |
| 261 | ++ | | | ++ | | | | | | | | | |
| 262 | ++ | | | ++ | | | | | | | | | |
| 263 | ++ | | | ++ | | | | | | | | | |
| 278 | ++ | | | ++ | | | | | | | | | |
| 279 | ++ | | | ++ | | | | | | | | | |
| 280 | + | | | + | | | | | | | | | |
| 283 | + | | | + | | | | | | | | | |
| 284 | + | | | + | | | | | | | | | |
| 285 | + | + | | + | | | | | | | | | |
| 286 | ++ | + | ++ | ++ | +++ | +++ | ++ | | +++ | | | | |
| 291 | | +++ | | | +++ | | | | | | | | |
| 306 | | + | | | | | | | | | | | |
| 307 | | + | | | + | | | | | | | | |
| 309 | | ++ | | | ++ | | | | | | | | |
| 310 | | +++ | | | +++ | | | | | | | | |
| 313 | | +++ | | | +++ | | | | | | | | |
| 314 | | ++ | | | ++ | | | | | | | | |
| 315 | | | | | | | | ++ | | + | | | |
| 318 | | +++ | | | +++ | | | | | | | | |
| 319 | | +++ | | | +++ | | | | | | | | |
| 320 | | ++ | | | +++ | | | | | | | | |
| 322 | | + | | | + | | | | | | | | |
| 323 | | + | | | + | | | | | | | | |
| 324 | | + | | | + | | | | | | | | |
| 325 | | + | | | + | | | | | | | | |
| 326 | | + | | | + | | | | | | | | |
| 327 | | + | | | + | | | | | | | | |
| 328 | | + | | | + | | | | | | | | |
| 329 | | + | | | + | | | | | | | | |
| 330 | | + | | | | | | | | | | | |
| 331 | | + | | | | | | | | | | | |
| 339 | | + | | | + | | | | | | | | |
| 340 | | ++ | | | ++ | | | | | | | | |
| 341 | | ++ | | | ++ | | | | | | | | |
| 342 | | ++ | | | +++ | | | | | | | | |
| 343 | | ++ | | | ++ | | | | | | | | |
| 344 | | + | | | + | | | | | | | | |

+++ means <1 μM
++ means ≧1 μM and <20 μM
+ means ≧20 μM

1. A compound of the formula 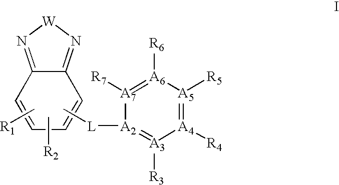
or a pharmaceutically acceptable salt, hydrate, solvate, polymorph, atrophisomer, N-oxide, or prodrug thereof, wherein
(A):
L is covalent bond, —N(H)—, —S—, —SO₂—, —SO₃—, —S(O)—, —SO₂N(H)—, —SO₂N(H)—C₁-C₆ alkyl, —N(H)—C(O)—, —N(H)—C(O)—N(H)—, —N(H)SO₂—, —N(H)(C₁-C₆ alkyl)SO₂—;
W is —O— or —S(O)₀₋₂;
A₂, A₃, A₄, A₅, A₆ and A₇ are independently carbon or nitrogen provided that when any one of A₂, A₃, A₄, A₅, A₆ and A₇ is nitrogen the R group attached to it is absent, or when any one of A₂, A₃, A₄, A₅, A₆ and A₇ is nitrogen and the R moiety attached to it is present, then the nitrogen has a positive charge and is a quaternary ammonium;
R₁ and R₂ are independently —H, —NO₂, —OH, —CN, —SH, —S—C₁-C₆ alkyl-aryl, —S-aryl, —S-heteroaryl, S—C₁-C₆-alkyl-heteroaryl, —O—C₁-C₆ alkyl-aryl, —O—C₁-C₆ alkyl-heteroaryl, —O-aryl, —O-heteroaryl —SO₂—OH, —S(O)—H, chloro, bromo, fluoro, iodo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, —N(R₈)-Z, aryl, heterocyclyl, or heteroaryl, wherein each of the aryl, heterocyclyl and heteroaryl is optionally substituted with 1 to 3 groups selected from —H, —OH, —SH, C₁-C₆ alkyl, C₁-C₆ alkoxy, mono- to per-halogenated C I—C₆ alkyl, —NH₂, mono- or di-(C₁-C₆ alkyl) amino, —NO₂, halo, —CN, or aryl or heteroaryl each optionally substituted with mono- to per-halogenated C₁-C₆ alkyl, —OH, or —CN;
R₈ is —H, C₁-C₆ alkyl, or C₁-C₆ alkoxy;
R₉ is —H or C₁-C₆ alkyl;
Z is aryl, heteroaryl, or —C(O)—CO—C₆ alkyl, —C(O)-heteroaryl, —C(O)-aryl, —S(O)₂-aryl, S(O)₂-heterocyclyl, —S(O)₂-heteroaryl, —C₁-C₆ alkyl-O—C(O)—C₁-C₆ alkyl, wherein each of the aryl, heterocyclyl, and heteroaryl is independently optionally substituted with 1-3 groups selected from —H, —NO₂, —CN, chloro, bromo, fluoro, iodo, C₁-C₆ alkyl, halo-C₁-C₆ alkyl, C₁-C₆ alkoxy, —NH₂, mono- or di-(C₁-C₆ alkyl) amino, mono- to per-halogenated C₁-C₆ alkoxy, and —C(O)—OR₉;
R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—R₉, —C(O)—OR₉, mono- to per-halogenated C₁-C₆ alkoxy, or mono- to per-halogenated C₁-C₆ alkyl; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—R₉, or —C(O)—OR₉;
R₅ is —H, halo, —NO₂, —CN, C₁-C₆ alkyl, C₁-C₆ alkoxy, heterocyclyl-S(O)₂-heteroaryl, aryl-S—C₁-C₆ alkyl, mono- to per-halogenated C₁-C₆ alkyl, —NH₂, mono- or di-(C₁-C₆ alkyl) amino, —C(O)—R₉, —C(O)—OR₉, —N(R₉)—C(O)R₉, —O-(halo C₁-C₆ alkyl), —O-Z; or
R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C I—C₆ alkyl, —NH₂, mono- or di-(C₁-C₆ alkyl) amino, or —C(O)—OR₉; and
R₆ and R₇ are independently —H, —NO₂, —CN, halo, C₁-C₆ alkyl, C₁-C₆ alkoxy optionally substituted with aryl or heteroaryl, mono- to per-halogenated C₁-C₆ alkyl, mono- to per-halogenated C₁-C₆ alkoxy, —NH₂, mono- or di-(C₁-C₆ alkyl) amino, —C(O)—OR₉, aryl, or heteroaryl;
(B):
provided that the compound is not one in which
(C):
W is —O—, and
a) R₁, R₂, R₃, R₄, R₆ and R₇ are —H, and L is —SO₂N(H)— attached at the 4 position of the benzoxadiazolyl ring, and R₅ is —C(O)—OCH₃ or —NH—C(O)CH₃; or —OCF₃;
b) R₁, R₂, R₆ and R₇ are —H, and L is —SO₂N(H)— attached at the 4 position of the berizoxadiazolyl ring, and R₃, R₄ and R₅ are —F;
c) R₂, R₅, R₆ and R₇ are —H, and L is —S— attached at the 4 position of the benzoxadiazolyl ring, and R₁ is —NO₂ and R₃ and R₄ together with the carbon atoms to which they are attached form a pyridinyl group;
d) R₂-R₇ are —H, and L is —S— attached at the 4 position of the benzoxadiazolyl ring, and R₁ is —H or NO₂;
e) R₂-R₇ are —H, and L is —S— or —SO₂N(H)— attached at the 4 position of the benzoxadiazolyl ring, and R₁ is —NH₂;
f) R₂-R₆ are —H, R₇ is —C(O)OH, and L is —S— attached at the 4 position of the benzoxadiazolyl ring, and R₁ is —NO₂;
g) R₁ is —NO₂, L is —S— attached at the 4 or 6 position of the benzoxadiazolyl ring, and A₂, A₃, A₄, A₅, A₆ and A₇ are carbon, and R₂, R₃, R₄, R₅, R₆ and R₇ are —H;
h) R₂-R₇ are —H, and L is —SO₂N(H)— attached at the 4 position of the benzoxadiazolyl ring, and R₁ is —F or —N(CH₃)₂; or
i) R₂, R₃, R₄ and R₇ are —H, R₁ is —NO₂, R₆ is —Cl, and L is —S— attached at the 4 position of the benzoxadiazolyl ring, and R₅ is —CH₃, —O—CH₃ or —Br.

2. A compound of the formula 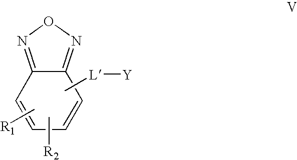
or a pharmaceutically acceptable salt, hydrate, solvate, polymorph, atrophisomer, N-oxide, or prodrug thereof, wherein
L′ is —SO₂-aryl, —SO₂-heteroaryl, —SO₂-heterocyclyl, —N(H)-heterocyclyl, —N(H)-heteroaryl, —N(H)C(O)-heterocyclyl, —N(H)C(O)-heteroaryl, C₃-C₆ cycloalkenyl, —S-heteroaryl, aryl, heteroaryl or heterocyclyl, wherein each of the aryl, heterocyclyl and heteroaryl is optionally substituted with 1 to 3 groups selected from —H, —OH, —S-heteroaryl, —S(O)₂-heteroaryl, heteroaryl, —O-heteroaryl, C₁-C₆ alkyl, C₁-C₆ alkoxy, C₁-C₆ alkyl, —C(O)—OR₉, —N(R₉)—C(O)R₉, —O—(C₁-C₆ alkyl), —NH₂, mono- or di-(C₁-C₆ alkyl) amino, —NO₂, halo, or —CN, or spiro-substituted with heterocyclyl or heteroaryl, each of which is optionally substituted with —H, OH, C I—C₆ alkyl, C₁-C₆ alkoxy, or oxo;
R₁ and R₂ are independently —H, —NO₂, —OH, —CN, halo, C₁-C₆ alkyl, C₁-C₆ alkoxy, mono- to per-halogenated C₁-C₆ alkyl, heterocyclyl, heterocyclyl-O-aryl, —NH₂, mono- or di-(C I—C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN; or
R₁ and R₂ together with the carbon atoms to which they are attached form an aryl group optionally substituted with 1 to 3 groups selected from —OH, halo, C₁-C₆-alkyl, C₁-C₆-alkoxy, —NH₂. and —NO₂;
Y is —H, —NO₂, —OH, —CN, halo, C₁-C₆ alkyl, C₁-C₆ alkoxy, —NH₂, mono- or di-(C₁-C₆ alkyl) amino, —C(O)—OR₉, —C(O)R₉, —N(R₈)-Z, C₁-C₆ alkyl-aryl, C₁-C₆ alkyl-heterocyclyl, C₁-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, —O-aryl, aryl, heterocyclyl, heteroaryl, or -Z₁-S(O)₂-Z₂ where Z, and Z₂ are independently aryl or heteroaryl, wherein each of the aryl, heterocyclyl and heteroaryl is optionally substituted with 1 to 3 groups selected from —H, C₁-C₆ alkyl, C₁-C₆ alkoxy, mono- to per-halogenated C₁-C₆ alkyl, —C(O)—OR₉, —N(R₉)—C(O)R₉, —O-(halo C₁-C₆ alkyl), —NH₂, mono- or di-(C₁-C₆ alkyl) amino, —NO₂, halo, or —CN;
R₈ is —H, C₁-C₆ alkyl, or C₁-C₆ alkoxy;
R₉ is —H, C₁-C₆ alkyl or C₃-C₆ cycloalkyl; and
Z is —C₁-C₆ alkyl-O—C(O)—C₁-C₆ alkyl, aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

3. A compound according to claim 2 of the formula 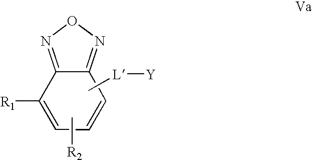
or a pharmaceutically acceptable salt, hydrate, solvate, polymorph, atrophisomer, N-oxide, and prodrug thereof.

4. A compound according to claim 2 of the formula 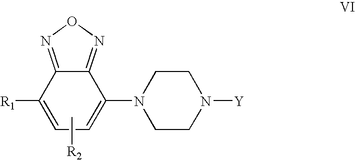
or a pharmaceutically acceptable salt, hydrate, solvate, polymorph, atrophisomer, N-oxide, or prodrug thereof, wherein
R₁ and R₂ are independently —H, —NO₂, —OH, —CN, halo, C₁-C₆ alkyl, C₁-C₆ alkoxy, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, —NH₂, mono- or di-(C₁-C₆ alkyl) amino, —NO₂, halo, or —CN;
Y is —H, —NO₂, —OH, —CN, halo, C₁-C₆ alkyl, C₁-C₆ alkoxy, —NH₂, mono- or di-(C₁-C₆ alkyl) amino, —C(O)—OR₉, —C(O)R₉, —N(R₈)-Z, C₀-C₆ alkyl-aryl, C₀-C₆ alkyl-heterocyclyl, C₀-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, aryl, heterocyclyl or heteroaryl, wherein each of the aryl, heterocyclyl and heteroaryl is optionally substituted with 1 to 3 groups selected from —H, C₁-C₆ alkyl, C₁-C₆ alkoxy, mono- to per-halogenated C₁-C₆ alkyl, —C(O)—OR₉, —N(R₉)—C(O)R₉, —O-(halo C₁-C₆ alkyl), —NH₂, mono- or di-(C₁-C₆ alkyl) amino, —NO₂, halo, or —CN;
R₈ is —H, C₁-C₆ alkyl, or C₁-C₆ alkoxy;
R₉ is —H, C₁-C₆ alkyl or C₃-C₆ cycloalkyl; and
Z is aryl, heteroaryl, —C(O)-aryl, or —C(O)-heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

5. A compound according to claim 3 of the formula 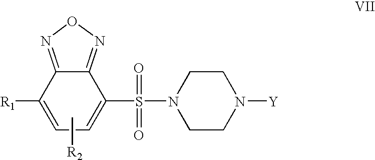
or a pharmaceutically acceptable salt, hydrate, solvate, polymorph, atrophisomer, N-oxide, or prodrug thereof, wherein
R₁ and R₂ are independently —H, —NO₂, —OH, —CN, halo, C₁-C₆ alkyl, C₁-C₆ alkoxy, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, —NH₂, mono- or di-(C₁-C₆ alkyl) amino, —NO₂, halo, or —CN;
Y is —H, —NO₂, —OH, —CN, halo, C₁-C₆ alkyl, C₁-C₆ alkoxy, —NH₂, mono- or di-(C₁-C₆ alkyl) amino, —C(O)—OR₉, —C(O)R₉, —N(R₈)-Z, C₁-C₆ alkyl-aryl, C₁-C₆ alkyl-heterocyclyl, C₁-C₆ alkyl-heteroaryl, C₀-C₆ alkyl-C(O)-aryl, C₀-C₆ alkyl-C(O)-heterocyclyl, C₀-C₆ alkyl-C(O)-heteroaryl, aryl, heterocyclyl or heteroaryl, wherein each of the aryl, heterocyclyl and heteroaryl is optionally substituted with 1 to 3 groups selected from —H, C₁-C₆ alkyl, C₁-C₆ alkoxy, halo-C₁-C₆ alkyl, —C(O)—OR₉, —N(R₉)—C(O)R₉, —O-(halo C₁-C₆ alkyl), —NH₂, mono- or di-(C₁-C₆ alkyl) amino, —NO₂, halo, or —CN;
R₈ is —H, C₁-C₆ alkyl, or C₁-C₆ alkoxy;
R₉ is —H, C₁-C₆ alkyl or C₃-C₆ cycloalkyl; and
Z is aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

6. A compound of the formula 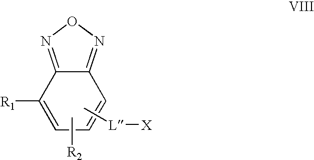
or a pharmaceutically acceptable salt, hydrate, solvate, polymorph, atrophisomer, N-oxide, or prodrug thereof, wherein
L″ is —S— or —SO₂N(R₉)—C₀-C₆ alkyl;
X is C₁-C₆ alkyl, halo, C₃-C₆ cycloalkyl, C₁-C₆ alkyl-C₁-C₆ alkoxy, C₁-C₆ alkoxy, mono- or di-(C₁-C₆ alkyl) amino, aryl, heterocyclyl, C₃-C₆ cycloalkyl, or heteroaryl, wherein each of the alkyl, aryl, heterocyclyl and heteroaryl is optionally substituted with 1 to 3 groups selected from —H, oxo, —O—C₀-C₆ alkyl-aryl, —O—SO₂-heteroaryl wherein the heteroaryl is optionally substituted with halo, —OH, C₁-C₆ alkyl, C₁-C₆ alkoxy, —NH₂, mono- or di-(C₁-C₆ alkyl) amino, —NO₂, halo, or —CN;
R₁ and R₂ are independently —H, —NO₂, —OH, —CN, halo, C₁-C₆ alkyl, C₁-C₆ alkoxy, —NH₂, —S-heteroaryl, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, —NH₂, mono- or di-(C₁-C₆ alkyl) amino, —NO₂, halo, or —CN;
R₈ is —H, C₁-C₆ alkyl, or C₁-C₆ alkoxy;
R₉ is —H or C₁-C₆ alkyl; and
Z is —C₁-C₆ alkyl-O—C(O)—C₁-C₆ alkyl, aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉.

7. The compound according to claim 1 of the formula 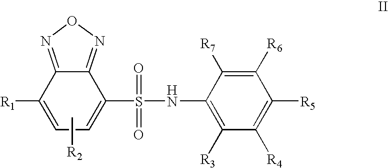
or a pharmaceutically acceptable salt, hydrate, solvate, polymorph, atrophisomer, N-oxide, or prodrug thereof.

8. The compound according to claim 7 of the formula 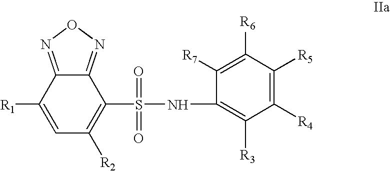
or a pharmaceutically acceptable salt, hydrate, solvate, polymorph, atrophisomer, N-oxide, or prodrug thereof, wherein
R₁ and R₂ are independently —H, —NO₂, —OH, —CN, halo, C₁-C₆ alkyl, C₁-C₆alkoxy, mono- to per-halogenated C₁-C₆ alkyl, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN;
R₈ is —H, C₁-C₆ alkyl, or C₁-C₆ alkoxy;
R₉ is —H or C₁-C₆ alkyl;
Z is aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉;
R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—OR₉, or mono- to per-halogenated C₁-C₆ alkyl; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉;
R₅ is —H, halo, —NO₂, —CN, C₁-C₆ alkyl, C₁-C₆ alkoxy, mono- to per-halogenated C₁-C₆ alkyl, —NH₂, mono- or di-(C₁-C₆ alkyl) amino, C(O)—OR₉, —N(R₉)—C(O)R₉, or —O-(halo C₁-C₆ alkyl); or
R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉; and
R₆ and R₇ are independently —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, aryl, or heteroaryl.

9. The compound according to claim 1 of the formula 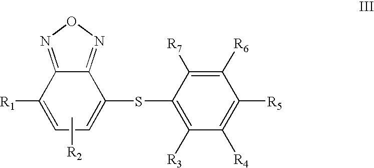
or a pharmaceutically acceptable salt, hydrate, solvate, polymorph, atrophisomer, N-oxide, or prodrug thereof

10. The compound according to claim 9 of the formula 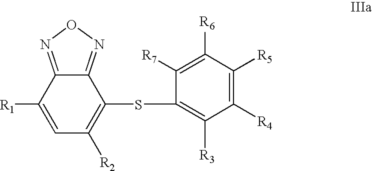
or a pharmaceutically acceptable salt, hydrate, solvate, polymorph, atrophisomer, N-oxide, or prodrug thereof, wherein
R₁ and R₂ are independently —H, —NO₂, —OH, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, —N(R₈)-Z, aryl, or heteroaryl, wherein each of the aryl and heteroaryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN;
R₈ is —H, C₁-C₆ alkyl, or C₁-C₆ alkoxy;
R₉ is —H or C₁-C₆ alkyl;
Z is aryl or heteroaryl wherein each of the aryl and heteroaryl is optionally substituted with —H, —NO₂, —CN, halo, C₁-C₆ alkyl, C₁-C₆ alkoxy, —NH₂, mono- or di-(C₁-C₆ alkyl) amino, or —C(O)—OR₉;
R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—OR₉, or mono- to per-halogenated C₁-C₆ alkyl; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉;
R₅ is —H, halo, —NO₂, —CN, C₁-C₆ alkyl, C₁-C₆ alkoxy, mono- to per-halogenated C₁-C₆ alkyl, —NH₂, mono- or di-(C₁-C₆ alkyl) amino, C(O)—OR₉, —N(R₉)—C(O)R₉, or —O-(halo C₁-C₆ alkyl); or
R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, or —C(O)—OR₉; and
R₆ and R₇ are independently —H, —NO₂, —CN, halo, C₁-C₆ alkyl, C₁-C₆ alkoxy, mono- to per-halogenated C₁-C₆ alkyl, —NH₂, mono- or di-(C₁-C₆ alkyl) amino, —C(O)—OR₉, aryl, or heteroaryl;
provided that:
when R₁ is —NO₂, then R₂, R₃, R₄, R₅, R₆ and R₇ are not —H.

11. The compound according to claim 1 of the formula 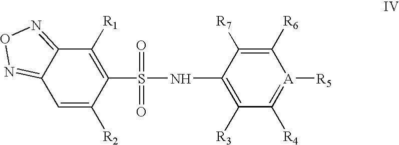
or a pharmaceutically acceptable salt, hydrate, solvate, polymorph, atrophisomer, N-oxide, or prodrug thereof, wherein
R₁ and R₂ are independently —H, C₁-C₄ alkyl, or C₁-C₄ alkoxy;
A is carbon or nitrogen provided that when A is nitrogen R₅ is absent;
R₃ and R₄ are independently —H, halo, C₁-C₄ alkyl, or C₁-C₄ alkoxy; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, halo, C₁-C₄ alkyl, or C₁-C₄ alkoxy;
R₅ is —H, halo, C₁-C₄ alkyl, or C₁-C₄ alkoxy;
R₅ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, halo, C₁-C₆ alkyl, C₁-C₆ alkoxy; and
R₆ and R₇ are independently —H, C₁-C₄ alkyl, or C₁-C₄ alkoxy.

12. A compound of the formula IXa or IXb: 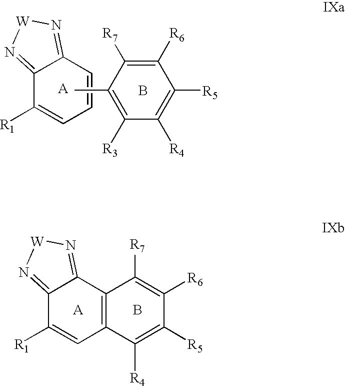
or pharmaceutically acceptable salt, hydrate, solvate, polymorph, atrophisomer, N-oxide, or prodrug thereof, wherein
W is O or S(O)₀₋₂;
R₁ is —H, —NO₂, —OH, —CN, halo, C₁-C₆ alkyl, C₁-C₆ alkoxy, mono- to per-halogenated C₁-C₆ alkyl, —NH₂, mono- or di-(C₁-C₆ alkyl) amino, or aryl, wherein the aryl is optionally substituted with 1 to 3 groups selected from —H, C₁-C₆ alkyl, C₁-C₆ alkoxy, mono- to per-halogenated C₁-C₆ alkyl, —NH₂, mono- or di-(C₁-C₆ alkyl) amino, —NO₂, halo, or —CN;
R₃ and R₄ are independently —H, halo, —NO₂, —CN, C₁-C₆ alkyl, C₁-C₆ alkoxy, —NH₂, mono- or di-(C₁-C₆ alkyl) amino, —C(O)—OR₉, or mono- to per-halogenated C₁-C₆ alkyl; or
R₃ and R₄ together with the carbon atoms to which they are attached form a heteroaryl, aryl, C₃-C₆ cycloalkyl, or heterocyclic group, wherein each of the heteroaryl, aryl, C₃-C₆ cycloalkyl, and heterocyclic group is optionally substituted with —H, —NO₂, —CN, halo, C₁-C₆ alkyl, C₁-C₆ alkoxy, mono- to per-halogenated C₁-C₆ alkyl, —NH₂, or mono- or di-(C₁-C₆ alkyl) amino;
R₅ is —H, halo, —NO₂, —CN, C₁-C₆ alkyl, C₁-C₆ alkoxy, —S—C₁-C₆ alkyl, mono- to per-halogenated C₁-C₆ alkyl, —NH₂, mono- or di-(C₁-C₆ alkyl) amino;
R₆ and R₇ are independently —H, —NO₂, —CN, halo, C₁-C₆ alkyl, C₁-C₆ alkoxy, mono- to per-halogenated C₁-C₆ alkyl, —NH₂, or mono- or di-(C₁-C₆ alkyl) amino.

13. A compound selected from the group consisting of:
methyl 4-{[(7-chloro-2,1,3-benzoxadiazol-4-yl)sulfonyl]amino}benzoate;
methyl 4-{[(7-{[4-(methoxycarbonyl)phenyl]amino}-2,1,3-benzoxadiazol-4-
yl)sulfonyl]amino}benzoate;
methyl 4-{[(5-{[4-(methoxycarbonyl)phenyl]amino}-2,1,3-benzoxadiazol-4-
yl)sulfonyl]amino}benzoate;
N-(2-methoxyphenyl)-2,1,3-benzoxadiazole-4-sulfonamide;
N-isoquinolin-5-yl-2,1,3-benzoxadiazole-4-sulfonamide;
N-(5-fluoro-2-methylphenyl)-2,1,3-benzoxadiazole-4-sulfonamide;
N-(3-chloro-4-fluorophenyl)-2,1,3-benzoxadiazole-4-sulfonamide;
N-(2,6-difluorophenyl)-2,1,3-benzoxadiazole-4-sulfonamide;
N-(2,4,6-trifluorophenyl)-2,1,3-benzoxadiazole-4-sulfonamide;
N-[2-methyl-3-(trifluoromethyl)phenyl]-2,1,3-benzoxadiazole-4-sulfonamide;
N-(4-iodo-3-methylphenyl)-2,1,3-benzoxadiazole-4-sulfonamide;
N-(4-fluoro-2-methylphenyl)-2,1,3-benzoxadiazole-4-sulfonamide;
N-(2,3-difluorophenyl)-2,1,3-benzoxadiazole-4-sulfonamide;
N-(2,5-dimethylphenyl)-2,1,3-benzoxadiazole-4-sulfonamide;
N-(3-fluorophenyl)-2,1,3-benzoxadiazole-4-sulfonamide;
N-(3,4-difluorophenyl)-2,1,3-benzoxadiazole-4-sulfonamide;
N-(2-methyl-1H-indol-5-yl)-2,1,3-benzoxadiazole-4-sulfonamide;
N-mesityl-2,1,3-benzoxadiazole-4-sulfonamide;
N-pyridin-4-yl-2,1,3-benzoxadiazole-5-sulfonamide;
N-(2,5-difluorophenyl)-2,1,3-benzoxadiazole-4-sulfonamide;
N-(3-chlorophenyl)-2,1,3-benzoxadiazole-4-sulfonamide;
N-benzyl-2,1,3-benzoxadiazole-5-sulfonamide;
N-(pyridin-2-ylmethyl)-2,1,3-benzoxadiazole-4-sulfonamide;
N-(2-methoxyethyl)-2,1,3-benzoxadiazole-4-sulfonamide;
N-(4-methoxyphenyl)-2,1,3-benzoxadiazole-4-sulfonamide;
N-(3,5-dichlorophenyl)-2,1,3-benzoxadiazole-4-sulfonamide;
N-(cyclohexylmethyl)-2,1,3-benzoxadiazole-4-sulfonamide;
N-(2-chloro-4-methylphenyl)-2,1,3-benzoxadiazole-4-sulfonamide;
N-(2-pyrrolidin-1-ylethyl)-2,1,3-benzoxadiazole-4-sulfonamide;
N-(5-chloro-2-methylphenyl)-2,1,3-benzoxadiazole-4-sulfonamide;
N-(2-iodophenyl)-2,1,3-benzoxadiazole-4-sulfonamide;
N-pyridin-2-yl-2,1,3-benzoxadiazole-4-sulfonamide;
N-(2-fluoroethyl)-2,1,3-benzoxadiazole-4-sulfonamide;
N-(2,5-dichlorophenyl)-2,1,3-benzoxadiazole-4-sulfonamide;
N-(2-fluoro-5-nitrophenyl)-2,1,3-benzoxadiazole-4-sulfonamide;
N-(4-{[3-chloro-5-(trifluoromethyl)pyridin-2-yl]oxy}phenyl)-2,1,3-
benzoxadiazole-4-sulfonamide;
dimethyl 2-[(2,1,3-benzoxadiazol-4-ylsulfonyl)amino]terephthalate;
N-quinolin-8-yl-2,1,3-benzoxadiazole-4-sulfonamide;
N-(4-nitrophenyl)-2,1,3-benzoxadiazole-4-sulfonamide;
N-(3-chloro-4-morpholin-4-ylphenyl)-2,1,3-benzoxadiazole-4-sulfonamide;
N-[2-(trifluoromethoxy)phenyl]-2,1,3-benzoxadiazole-4-sulfonamide;
N-pyridin-4-yl-2,1,3-benzoxadiazole-4-sulfonamide;
N-2,1,3-benzothiadiazol-4-yl-4-iodobenzenesulfonamide;
N-2,1,3-benzothiadiazol-4-yl-3,4-difluorobenzenesulfonamide;
N-2,1,3-benzothiadiazol-4-yl-3,4,5-trifluorobenzenesulfonamide;
tert-butyl 4-(7-nitro-2,1,3-benzoxadiazol-4-yl)piperazine-1-carboxylate;
N-2,1,3-benzothiadiazol-4-yl-3,5-dichloro-4-(2-chloro-4-
nitrophenoxy)benzenesulfonamide;
ethyl 3-[(2,1,3-benzoxadiazol-4-ylsulfonyl)amino]benzoate;
N-2,1,3-benzothiadiazol-4-yl-2-bromo-4,6-difluorobenzenesulfonamide;
N-2,1,3-benzothiadiazol-4-yl-3-chloro-4-fluorobenzenesulfonamide;
N-2,1,3-benzothiadiazol-4-yl-2-bromo-4-fluorobenzenesulfonamide;
N-2,1,3-benzoxadiazol-4-yl-4-{[3-chloro-5-(trifluoromethyl)pyridin-2-
yl]oxy}benzenesulfonamide;
N-2,1,3-benzoxadiazol-4-yl-3-chloro-4-(2,6-dichloro-4-
nitrophenoxy)benzenesulfonamide;
N-2,1,3-benzoxadiazol-4-yl-4-(pyridin-4-yloxy)benzenesulfonamide;
N-2,1,3-benzoxadiazol-4-yl-4-{[5,7-bis(trifluoromethyl)-1,8-naphthyridin-2-
yl]oxy}benzenesulfonamide;
4-nitro-7-(4-pyridin-2-ylpiperazin-1-yl)-2,1,3-benzoxadiazole;
N-2,1,3-benzoxadiazol-4-yl-3,5-dichloro-4-hydroxybenzenesulfonamide;
4-nitro-7-(4-pyridin-2-ylpiperazin-1-yl)-2,1,3-benzoxadiazole;
4-[4-(2-morpholin-4-ylethyl)piperazin-1-yl]-7-nitro-2,1,3-benzoxadiazole;
N-2,1,3-benzoxadiazol-4-yl-4-(pyridin-3-yloxy)benzenesulfonamide;
N-2,1,3-benzoxadiazol-4-yl-5-phenoxypyridine-2-sulfonamide;
4-(4-acetylpiperazin-1-yl)-7-nitro-2,1,3-benzoxadiazole;
N-2,1,3-benzothiadiazol-4-yl-4-fluorobenzenesulfonamide;
4-nitro-7-{4-[4-(trifluoromethyl)pyridin-2-yl]piperazin-1-yl}-2,1,3-
benzoxadiazole;
N-[3-chloro-4-(pyrimidin-2-yloxy)phenyl]-2,1,3-benzoxadiazole-4-sulfonamide;
N-[3-bromo-4-(4-fluorophenoxy)phenyl]-2,1,3-benzoxadiazole-4-sulfonamide;
N-2,1,3-benzoxadiazol-4-yl-4-phenoxybenzenesulfonamide;
N-2,1,3-benzothiadiazol-4-yl-4-phenoxybenzenesulfonamide;
4-{[4-(pyrimidin-2-yloxy)piperidin-1-yl]sulfonyl}-2,1,3-benzoxadiazole;
N-2,1,3-benzothiadiazol-4-yl-3,5-dichloro-4-hydroxybenzenesulfonamide;
N-2,1,3-benzothiadiazol-4-yl-3-bromobenzenesulfonamide;
N-2,1,3-benzothiadiazol-4-yl-4-(bromomethyl)benzenesulfonamide;
N-2,1,3-benzothiadiazol-4-yl-2,3,4-trifluorobenzenesulfonamide;
N-2,1,3-benzothiadiazol-4-yl-2-cyanobenzenesulfonamide;
N-[4-(4-nitrophenoxy)phenyl]-2,1,3-benzoxadiazole-4-sulfonamide;
N-(4-phenoxyphenyl)-2,1,3-benzoxadiazole-4-sulfonamide;
N-(4-{[4-(trifluoromethyl)pyridin-2-yl]oxy}phenyl)-2,1,3-benzoxadiazole-4-
sulfonamide;
N-[4-(4-chlorophenoxy)phenyl]-2,1,3-benzoxadiazole-4-sulfonamide;
4-{4-[4-chloro-3-(trifluoromethyl)phenyl]piperazin-1-yl}-7-nitro-2,1,3-
benzoxadiazole;
4-nitro-7-{4-[3-(trifluoromethyl)phenyl]piperazin-1-yl}-2,1,3-benzoxadiazole;
4-[4-(2-morpholin-4-yl-2-oxoethyl)piperazin-1-yl]-7-nitro-2,1,3-benzoxadiazole;
4-nitro-7-(4-pyridin-4-ylpiperazin-1-yl)-2,1,3-benzoxadiazole;
4-nitro-7-(1,3-(pyrimidin-2-yl)piperazin-1-yl)-2,1,3-benzoxadiazole
4-(4-methylpiperazin-1-yl)-7-nitro-2,1,3-benzoxadiazole;
N-methyl-N-[2-(methylamino)ethyl]-2,1,3-benzoxadiazole-4-sulfonamide;
4-nitro-7-piperazin-1-yl-2,1,3-benzoxadiazole;
N-2,1,3-benzothiadiazol-4-yl-3-chloro-4-[4-nitro-2-
(trifluoromethyl)phenoxy]benzenesulfonamide;
N-2,1,3-benzoxadiazol-4-yl-3-chloro-4-[4-nitro-2-
(trifluoromethyl)phenoxy]benzenesulfonamide;
N-2,1,3-benzothiadiazol-4-yl-5-phenoxypyridine-2-sulfonamide;
N-2,1,3-benzothiadiazol-4-yl-4-(pyridin-3-yloxy)benzenesulfonamide;
N-2,1,3-benzothiadiazol-4-yl-6-[2-chloro-4-(trifluoromethyl)phenoxy]pyridine-3-
sulfonamide;
2-[4-(7-nitro-2,1,3-benzoxadiazol-4-yl)piperazin-1-yl]nicotinonitrile;
2-[4-(2,1,3-benzoxadiazol-4-ylsulfonyl)piperazin-1-yl]nicotinonitrile;
6-[4-(2,1,3-benzoxadiazol-4-ylsulfonyl)piperazin-1-yl]nicotinonitrile;
6-[4-(7-nitro-2,1,3-benzoxadiazol-4-yl)piperazin-1-yl]nicotinonitrile;
7-{4-[4-(trifluoromethyl)pyridin-2-yl]piperazin-1-yl}-2,1,3-benzoxadiazol-4-
amine;
4-[(4-pyridin-2-ylpiperazin-1-yl)sulfonyl]-2,1,3-benzoxadiazole;
4-({4-[4-(trifluoromethyl)pyridin-2-yl]piperazin-1-yl}sulfonyl)-2,1,3-
benzoxadiazole;
4-({4-[3-(trifluoromethyl)phenyl]piperazin-1-yl}sulfonyl)-2,1,3-benzoxadiazole;
4-[(4-pyridin-4-ylpiperazin-1-yl)sulfonyl]-2,1,3-benzoxadiazole;
4-{[4-(2-morpholin-4-ylethyl)piperazin-1-yl]sulfonyl}-2,1,3-benzoxadiazole;
4-[(4-acetylpiperazin-1-yl)sulfonyl]-2,1,3-benzoxadiazole;
4-{[4-(2-morpholin-4-yl-2-oxoethyl)piperazin-1-yl]sulfonyl}-2,1,3-
benzoxadiazole;
4-[(4-pyrimidin-2-ylpiperazin-1-yl)sulfonyl]-2,1,3-benzoxadiazole;
4-[(4-pyrazin-2-ylpiperazin-1-yl)sulfonyl]-2,1,3-benzoxadiazole;
4-({4-[4-chloro-3-(trifluoromethyl)phenyl]piperazin-1-yl}sulfonyl)-2,1,3-
benzoxadiazole;
3-({7-[(5-methyl-1,3,4-thiadiazol-2-yl)thio]-4-nitro-2,1,3-benzoxadiazol-5-
yl}amino)propyl acetate;
3-{[4-nitro-7-(propylthio)-2,1,3-benzoxadiazol-5-yl]amino}propyl acetate;
3-{[7-(cyclohexylthio)-4-nitro-2,1,3-benzoxadiazol-5-yl]amino}propyl acetate;
3-({7-[(2-furylmethyl)thio]-4-nitro-2,1,3-benzoxadiazol-5-yl}amino)propyl
acetate;
ethyl [(6-{[3-(acetyloxy)propyl]amino}-7-nitro-2,1,3-benzoxadiazol-4-
yl)thio]acetate;
4-[4-(2,1,3-benzoxadiazol-4-ylsulfonyl)piperazin-1-yl]phenyl 2,1,3-
benzoxadiazole-4-sulfonate;
4-[4-(3,4-dimethylphenyl)piperazin-1-yl]-7-nitro-2,1,3-benzoxadiazole;
1-(7-nitro-2,1,3-benzoxadiazol-4-yl)-4-phenylpiperidine-4-carbonitrile;
(4-fluorophenyl)[1-(7-nitro-2,1,3-benzoxadiazol-4-yl)piperidin-4-yl]methanone;
4-nitro-7-(4-phenylpiperazin-1-yl)-2,1,3-benzoxadiazole;
4-(3,5-dimethylpiperidin-1-yl)-7-nitro-2,1,3-benzoxadiazole;
4-[4-(5-chloro-2-methylphenyl)piperazin-1-yl]-7-nitro-2,1,3-benzoxadiazole;
4-[4-(7-nitro-2,1,3-benzoxadiazol-4-yl)piperazin-1-yl]phenol;
4-nitro-7-[4-(4-nitrophenyl)piperazin-1-yl]-2,1,3-benzoxadiazole;
4-nitro-7-{4-[3-(trifluoromethyl)pyridin-2-yl]piperazin-1-yl}-2,1,3-
benzoxadiazole;
4-[(3-chlorophenyl)thio]-7-nitro-2,1,3-benzoxadiazole;
2-[(7-nitro-2,1,3-benzoxadiazol-4-yl)thio]ethanol;
4-[4-(3,5-dichloropyridin-4-yl)piperazin-1-yl]-7-nitro-2,1,3-benzoxadiazole;
2-methyl-4-[4-(7-nitro-2,1,3-benzoxadiazol-4-yl)piperazin-1-yl]quinoline;
ethyl 5-cyano-6-methyl-2-[4-(7-nitro-2,1,3-benzoxadiazol-4-yl)piperazin-1-
yl]nicotinate;
4-[4-(4-methylpyridin-2-yl)piperazin-1-yl]-7-nitro-2,1,3-benzoxadiazole;
2-[4-(7-nitro-2,1,3-benzoxadiazol-4-yl)piperazin-1-yl]quinoline;
4-[4-(3-methylpyridin-2-yl)piperazin-1-yl]-7-nitro-2,1,3-benzoxadiazole;
4-[(4-methylphenyl)thio]-7-nitro-2,1,3-benzoxadiazole;
4-(1,3-benzothiazol-2-ylthio)-7-nitro-2,1,3-benzoxadiazole;
4-(4-chlorophenyl-1-(7-nitro-2,1,3-benzoxadiazol-4-yl)piperidin-4-ol;
1-[1-(7-nitro-2,1,3-benzoxadiazol-4-yl)piperidin-4-yl]-1,3-dihydro-2H-
benzimidazol-2-one
4-(2,6-dimethylmorpholin-4-yl)-7-nitro-2,1,3-benzoxadiazole;
4-[4-(6-methylpyridin-2-yl)piperazin-1-yl]-7-nitro-2,1,3-benzoxadiazole;
4-({4-[3-chloro-5-(trifluoromethyl)pyridin-2-yl]piperazin-1-yl}sulfonyl)-7-
methoxy-2,1,3-benzoxadiazole;
4-({4-[3-chloro-5-(trifluoromethyl)pyridin-2-yl]piperazin-1-yl}sulfonyl)-7-
fluoro-2,1,3-benzoxadiazole;
4-fluoro-7-({4-[3-(trifluoromethyl)pyridin-2-yl]piperazin-1-yl}sulfonyl)-2,1,3-
benzoxadiazole;
4-{4-[3-(trifluoromethyl)pyridin-2-yl]piperazin-1-yl}-7-({4-[3-
(trifluoromethyl)pyridin-2-yl]piperazin-1-yl}sulfonyl)-2,1,3-benzoxadiazole;
methyl 4-{[(7-fluoro-2,1,3-benzoxadiazol-4-yl)sulfonyl]amino}benzoate;
1-[(7-chloro-2,1,3-benzoxadiazol-4-yl)sulfonyl]-4-(4-chlorophenyl)piperidin-4-
ol;
4-chloro-7-({4-[3-chloro-5-(trifluoromethyl)pyridin-2-yl]piperazin-1-
yl}sulfonyl)-2,1,3-benzoxadiazole;
2-{4-[(7-chloro-2,1,3-benzoxadiazol-4-yl)sulfonyl]piperazin-1-yl}nicotinonitrile;
4-{4-[(7-chloro-2,1,3-benzoxadiazol-4-yl)sulfonyl]piperazin-1-yl}phenyl 7-
chloro-2,1,3-benzoxadiazole-4-sulfonate;
4-{4-[(7-chloro-2,1,3-benzoxadiazol-4-yl)sulfonyl]piperazin-1-yl}phenol;
4-[4-({7-[4-(4-hydroxyphenyl)piperazin-1-yl]-2,1,3-benzoxadiazol-4-
yl}sulfonyl)piperazin-1-yl]phenol;
methyl 4-({[7-(quinolin-8-ylthio)-2,1,3-benzoxadiazol-4-
yl]sulfonyl}amino)benzoate;
4-(4-chlorophenyl)-1-{[7-(quinolin-8-ylthio)-2,1,3-benzoxadiazol-4-
yl]sulfonyl}piperidin-4-ol;
2-(4-{[7-(quinolin-8-ylthio)-2,1,3-benzoxadiazol-4-yl]sulfonyl}piperazin-1-
yl)nicotinonitrile;
8-{[7-({4-[3-chloro-5-(trifluoromethyl)pyridin-2-yl]piperazin-1-yl}sulfonyl)-
2,1,3-benzoxadiazol-4-yl]thio}quinoline;
methyl 4-{[(5-chloro-2,1,3-benzoxadiazol-4-yl)sulfonyl]amino}benzoate;
5-chloro-4-({4-[3-chloro-5-(trifluoromethyl)pyridin-2-yl]piperazin-1-
yl}sulfonyl)-2,1,3-benzoxadiazole;
2-{4-[(5-chloro-2,1,3-benzoxadiazol-4-yl)sulfonyl]piperazin-1-yl}benzonitrile;
5-{4-[4-chloro-3-(trifluoromethyl)phenyl]piperazin-1-yl}-4-({4-[4-chloro-3-
(trifluoromethyl)phenyl]piperazin-1-yl}sulfonyl)-2,1,3-benzoxadiazole;
5-chloro-4-({4-[4-chloro-3-(trifluoromethyl)phenyl]piperazin-1-yl}sulfonyl)-
2,1,3-benzoxadiazole;
2-(4-{[5-(quinolin-8-ylthio)-2,1,3-benzoxadiazol-4-yl]sulfonyl}piperazin-1-
yl)nicotinonitrile;
4-chloro-7-[(4-pyrimidin-2-ylpiperazin-1-yl)sulfonyl]-2,1,3-benzoxadiazole;
2-(2-{4-[(7-chloro-2,1,3-benzoxadiazol-4-yl)sulfonyl]piperazin-1-
yl}ethoxy)ethanol;
4-chloro-7-[(4-phenylpiperazin-1-yl)sulfonyl]-2,1,3-benzoxadiazole;
4-(4-phenylpiperazin-1-yl)-7-[(4-phenylpiperazin-1-yl)sulfonyl]-2,1,3-
benzoxadiazole;
1-{1-[(7-chloro-2,1,3-benzoxadiazol-4-yl)sulfonyl]piperidin-4-yl}-1,3-dihydro-
2H-benzimidazol-2-one;
4-chloro-7-[(4-methylpiperazin-1-yl)sulfonyl]-2,1,3-benzoxadiazole;
1-(1-{[7-(quinolin-8-ylthio)-2,1,3-benzoxadiazol-4-yl]sulfonyl}piperidin-4-yl)-
1,3-dihydro-2H-benzimidazol-2-one;
8-({7-[(4-methylpiperazin-1-yl)sulfonyl]-2,1,3-benzoxadiazol-4-
yl}thio)quinoline;
4-chloro-7-[(3,5-dimethylpiperidin-1-yl)sulfonyl]-2,1,3-benzoxadiazole;
4-(3,5-dimethylpiperidin-1-yl)-7-[(3,5-dimethylpiperidin-1-yl)sulfonyl]-2,1,3-
benzoxadiazole;
4-chloro-7-{[4-(6-methylpyridin-2-yl)piperazin-1-yl]sulfonyl}-2,1,3-
benzoxadiazole;
7-amino-N-(2-chloropyridin-4-yl)-2,1,3-benzoxadiazole-4-sulfonamide;
8-[(7-{[4-(6-methylpyridin-2-yl)piperazin-1-yl]sulfonyl}-2,1,3-benzoxadiazol-4-
yl)thio]quinoline;
8-({7-[(3,5-dimethylpiperidin-1-yl)sulfonyl]-2,1,3-benzoxadiazol-4-
yl}thio)quinoline;
7-chloro-N-(6-ethoxy-1,3-benzothiazol-2-yl)-2,1,3-benzoxadiazole-4-
sulfonamide;
1-[(7-hydroxy-2,1,3-benzoxadiazol-4-yl)sulfonyl]-4-phenylpiperidine-4-
carbonitrile;
5-chloro-4-[(4-pyrazin-2-ylpiperazin-1-yl)sulfonyl]-2,1,3-benzoxadiazole;
4-[(4-acetylpiperazin-1-yl)sulfonyl]-5-chloro-2,1,3-benzoxadiazole;
5-chloro-4-{[4-(6-methylpyridin-2-yl)piperazin-1-yl]sulfonyl}-2,1,3-
benzoxadiazole;
5-[4-(6-methylpyridin-2-yl)piperazin-1-yl]-4-{[4-(5-methylpyridin-2-
yl)piperazin-1-yl]sulfonyl}-2,1,3-benzoxadiazole;
5-chloro-4-[(2,6-dimethylmorpholin-4-yl)sulfonyl]-2,1,3-benzoxadiazole;
5-(2,6-dimethylmorpholin-4-yl)-4-[(2,6-dimethylmorpholin-4-yl)sulfonyl]-2,1,3-
benzoxadiazole;
8-[(4-{[4-(6-methylpyridin-2-yl)piperazin-1-yl]sulfonyl}-2,1,3-benzoxadiazol-5-
yl)thio]quinoline;
8-({4-[(4-pyrazin-2-ylpiperazin-1-yl)sulfonyl]-2,1,3-benzoxadiazol-5-
yl}thio)quinoline;
6-{4-[(5-chloro-2,1,3-benzoxadiazol-4-yl)sulfonyl]piperazin-1-yl}nicotinonitrile;
6-[4-({5-[4-(5-cyanopyridin-2-yl)piperazin-1-yl]-2,1,3-benzoxadiazol-4-
yl}sulfonyl)piperazin-1-yl]nicotinonitrile;
4-(2,6-dimethylmorpholin-4-yl)-7-[(2,6-dimethylmorpholin-4-yl)sulfonyl]-2,1,3-
benzoxadiazole;
6-(4-{[5-(quinolin-8-ylthio)-2,1,3-benzoxadiazol-4-yl]sulfonyl}piperazin-1-
yl)nicotinonitrile;
8-({7-[(2,6-dimethylmorpholin-4-yl)sulfonyl]-2,1,3-benzoxadiazol-4-
yl}thio)quinoline;
8-({4-[(4-acetylpiperazin-1-yl)sulfonyl]-2,1,3-benzoxadiazol-5-yl}thio)quinoline;
methyl 4-({[5-(quinolin-8-ylthio)-2,1,3-benzoxadiazol-4-
yl]sulfonyl}amino)benzoate;
8-({4-[(2,6-dimethylmorpholin-4-yl)sulfonyl]-2,1,3-benzoxadiazol-5-
yl}thio)quinoline;
8-({7-[(4-phenylpiperazin-1-yl)sulfonyl]-2,1,3-benzoxadiazol-4-
yl}thio)quinoline;
7-chloro-N-1H-indazol-5-yl-2,1,3-benzoxadiazole-4-sulfonamide;
5-chloro-N-1H-indazol-6-yl-2,1,3-benzoxadiazole-4-sulfonamide;
7-chloro-N-1H-indazol-6-yl-2,1,3-benzoxadiazole-4-sulfonamide;
1-[(7-chloro-2,1,3-benzoxadiazol-4-yl)sulfonyl]-1H-indazol-6-amine;
N-1H-indazol-5-yl-7-nitro-2,1,3-benzoxadiazol-4-amine;
N-1H-indazol-6-yl-7-nitro-2,1,3-benzoxadiazol-4-amine;
N-1H-indazol-5-yl-7-(quinolin-8-ylthio)-2,1,3-benzoxadiazole-4-sulfonamide;
4-chloro-7-{[4-(pyrimidin-2-yloxy)piperidin-1-yl]sulfonyl}-2,1,3-
benzoxadiazole;
4-[(7-{[4-(pyrimidin-2-yloxy)piperidin-1-yl]sulfonyl}-2,1,3-benzoxadiazol-4-
yl)thio]phenol;
1-{1-[(7-chloro-2,1,3-benzoxadiazol-4-yl)sulfonyl]-4-phenylpiperidin-4-
yl}ethanone;
4-(4-chlorophenyl)-1-({7-[(4-hydroxyphenyl)thio]-2,1,3-benzoxadiazol-4-
yl}sulfonyl)piperidin-4-ol;
5-chloro-4-({4-[2-(trifluoromethyl)phenoxy]piperidin-1-yl}sulfonyl)-2,1,3-
benzoxadiazole;
4-chloro-7-({4-[2-(trifluoromethyl)phenoxy]piperidin-1-yl}sulfonyl)-2,1,3-
benzoxadiazole;
1-[1-(7-nitro-2,1,3-benzoxadiazol-4-yl)-4-phenylpiperidin-4-yl]ethanone;
4-fluoro-7-({4-[2-(trifluoromethyl)phenoxy]piperidin-1-yl}sulfonyl)-2,1,3-
benzoxadiazole;
4-nitro-7-{4-[2-(trifluoromethyl)phenoxy]piperidin-1-yl}-2,1,3-benzoxadiazole;
5-chloro-4-[[(1,3-pyrimidin-2-yloxy)piperidin-1-yl]sulfonyl]piperazin-1-yl]-
2,1,3-benzoxadiazole;
4-[4-(1,3-pyrimidin-2-yloxy)piperazin-1-ylsulsonyl]-2,1,3-benzoxadiazole;
5-[4-(1,3-pyrimidin-2-yloxy)piperidin-1-yl]-4-{[4-(1,3-pyrimidin-2-
yloxy)piperidin-1-yl]sulfonyl}-2,1,3-benzoxadiazole;
4-nitro-7-thiomorpholin-4-yl-2,1,3-benzoxadiazole;
2-[(7-nitro-2,1,3-benzoxadiazol-4-yl)thio]quinoline;
4-({4-[2-(trifluoromethyl)phenoxy]piperidin-1-yl}sulfonyl)-2,1,3-
benzothiadiazole;
4-(4,4-dimethylpiperidin-1-yl)-7-nitro-2,1,3-benzoxadiazole;
4-[(4,4-dimethylpiperidin-1-yl)sulfonyl]-7-fluoro-2,1,3-benzoxadiazole;
5-chloro-4-[(4,4-dimethylpiperidin-1-yl)sulfonyl]-2,1,3-benzoxadiazole;
4-(4-chlorophenyl)-1-{[7-(pyridin-4-ylthio)-2,1,3-benzoxadiazol-4-
yl]sulfonyl}piperidin-4-ol;
4-chloro-7-[4,4-dimethylpiperidin-1-yl)sulfonyl]-2,1,3-benzoxadiazole;
methyl 4-({[7-quinolin-2-ylthio)-2,1,3-benzoxadiazol-4-
yl)sulfonyl}amino)benzoate;
4-({7-[(4-phenylpiperazin-1-yl)sulfonyl]-2,1,3-benzoxadiazol-4-yl}thio)phenol;
methyl 4-{[(7-thiomorpholin-4-yl-2,1,3-benzoxadiazol-4-
yl)sulfonyl]amino}benzoate;
4-[(4-phenylpiperazin-1-yl)sulfonyl]-7-(pyridin-4-ylthio)-2,1,3-benzoxadiazole;
2-({7-[(4-phenylpiperazin-1-yl)sulfonyl]-2,1,3-benzoxadiazol-4-
yl}thio)quinoline;
4-(1-naphthylthio)-7-[(4-phenylpiperazin-1-yl)sulfonyl]-2,1,3-benzoxadiazole;
4-[(2-methoxyphenyl)thio]-7-[(4-phenylpiperazin-1-yl)sulfonyl]-2,1,3-
benzoxadiazole;
8-({7-[(4-phenylpiperazin-1-yl)sulfonyl]-2,1,3-benzoxadiazol-4-
yl}oxy)quinoline;
methyl 4-({[7-(pyridin-4-ylthio)-2,1,3-benzoxadiazol-4-
yl]sulfonyl}amino)benzoate;
1′-(2,1,3-benzothiadiazol-4-ylsulfonyl)-3H-spiro[2-benzofuran-1,4′-piperidine];
1′-[(5-chloro-2,1,3-benzoxadiazol-4-yl)sulfonyl]-3H-spiro[2-benzofuran-1,4′-
piperidine];
1′-[(7-fluoro-2,1,3-benzoxadiazol-4-yl)sulfonyl]-3H-spiro[2-benzofuran-1,4′-
piperidine];
1′-[(7-chloro-2,1,3-benzoxadiazol-4-yl)sulfonyl]-3H-spiro[2-benzofuran-1,4′-
piperidine];
4-[(4-methylpiperazin-1-yl)sulfonyl]-7-(2-naphthylthio)-2,1,3-benzoxadiazole;
7-chloro-N-(3,4-difluorophenyl)-2,1,3-benzoxadiazole-4-sulfonamide;
7-chloro-N-(3-fluorophenyl)-2,1,3-benzoxadiazole-4-sulfonamide;
N-(3,4-difluorophenyl)-7-[(3,4-difluorophenyl)amino]-2,1,3-benzoxadiazole-4-
sulfonamide;
8-({7-[(4-methylpiperazin-1-yl)sulfonyl]-2,1,3-benzoxadiazol-4-
yl}thio)quinoline;
4-({7-[(4-methylpiperazin-1-yl)sulfonyl]-2,1,3-benzoxadiazol-4-yl}thio)phenol;
4-[(4-methylpiperazin-1-yl)sulfonyl]-7-(1-naphthylthio)-2,1,3-benzoxadiazole;
2-({7-[(4-methylpiperazin-1-yl)sulfonyl]-2,1,3-benzoxadiazol-4-
yl}thio)quinoline;
7-chloro-N-(5-chloro-2-methoxyphenyl)-2,1,3-benzoxadiazole-4-sulfonamide;
4-[(2-methoxyphenyl)thio]-7-[(4-methylpiperazin-1-yl)sulfonyl]-2,1,3-
benzoxadiazole;
7-chloro-N-(2,3-difluorophenyl)-2,1,3-benzoxadiazole-4-sulfonamide;
7-fluoro-N-(3-fluorophenyl)-2,1,3-benzoxadiazole-4-sulfonamide;
N-(3-fluorophenyl)-7-[(3-fluorophenyl)amino]-2,1,3-benzoxadiazole-4-
sulfonamide;
N-(2,3-difluorophenyl)-7-fluoro-2,1,3-benzoxadiazole-4-sulfonamide;
N-(4-fluoro-2-methylphenyl)-7-[(4-fluoro-2-methylphenyl)amino]-2,1,3-
benzoxadiazole-4-sulfonamide;
7-fluoro-N-(4-fluoro-2-methylphenyl)-2,1,3-benzoxadiazole-4-sulfonamide;
methyl 4-({[7-(quinolin-8-yloxy)-2,1,3-benzoxadiazol-4-
yl]sulfonyl}amino)benzoate;
benzyl 4-(7-nitro-2,1,3-benzoxadiazol-4-yl)piperazine-1-carboxylate;
7-{4-[3-chloro-5-(trifluoromethyl)pyridin-2-yl]piperazin-1-yl}-2,1,3-
benzoxadiazol-4-amine;
4-nitro-7-[4-(pyrimidin-2-yloxy)piperidin-1-yl]-2,1,3-benzoxadiazole;
N-(7-{4-[3-chloro-5-(trifluoromethyl)pyridin-2-yl]piperazin-1-yl}-2,1,3-
benzoxadiazol-4-yl)thiophene-2-carboxamide;
4-{4-[3-chloro-5-(trifluoromethyl)pyridin-2-yl]piperazin-1-yl}-7-morpholin-4-yl-
2,1,3-benzoxadiazole;
4-nitro-7-(4-pyridin-4-ylpiperidin-1-yl)-2,1,3-benzoxadiazole;
1′-(7-nitro-2,1,3-benzoxadiazol-4-yl)-3H-spiro[2-benzofuran-1,4′-piperidine];
4-(3,5-dimethylphenyl)-2,1,3-benzoxadiazole;
4-(3,5-dimethylphenyl)-7-nitro-2,1,3-benzoxadiazole;
4-nitro-7-[4-(pyridin-2-yloxy)piperidin-1-yl]-2,1,3-benzoxadiazole;
4-nitro-7-[4-(pyridin-2-ylthio)piperidin-1-yl]-2,1,3-benzoxadiazole;
4-nitro-7-[4-(pyridin-2-ylsulfonyl)piperidin-1-yl]-2,1,3-benzoxadiazole;
8-[(7-nitro-2,1,3-benzoxadiazol-4-yl)sulfonyl]quinoline;
8-[(7-nitro-2,1,3-benzoxadiazol-4-yl)sulfinyl]quinoline;
7-{4-[4-chloro-3-(trifluoromethyl)phenyl]piperazin-1-yl}-N,N-diethyl-4-nitro-
2,1,3-benzoxadiazol-5-amine;
7-{4-[3-chloro-5-(trifluoromethyl)pyridin-2-yl]piperazin-1-yl}-N,N-diethyl-4-
nitro-2,1,3-benzoxadiazol-5-amine;
2-{4-[6-(diethylamino)-7-nitro-2,1,3-benzoxadiazol-4-yl]piperazin-1-
yl}nicotinonitrile;
7-{4-[4-chloro-3-(trifluoromethyl)phenyl]piperazin-1-yl}-4-nitro-5-piperidin-1-
yl-2,1,3-benzoxadiazole;
7-{4-[3-chloro-5-(trifluoromethyl)pyridin-2-yl]piperazin-1-yl}-4-nitro-5-
piperidin-1-yl-2,1,3-benzoxadiazole;
2-[4-(7-nitro-6-piperidin-1-yl-2,1,3-benzoxadiazol-4-yl)piperazin-1-
yl]nicotinonitrile;
N,N-diethyl-4-nitro-7-(1′H,3H-spiro[2-benzofuran-1,4′-piperidin]-1′-yl)-2,1,3-
benzoxadiazol-5-amine;
1-[6-(diethylamino)-7-nitro-2,1,3-benzoxadiazol-4-yl]-4-phenylpiperidin-4-ol;
1′-(7-nitro-6-piperidin-1-yl-2,1,3-benzoxadiazol-4-yl)-3H-spiro[2-benzofuran-
1,4′-piperidine];
1-(7-nitro-6-piperidin-1-yl-2,1,3-benzoxadiazol-4-yl)-4-phenylpiperidin-4-ol;
1′-(7-nitro-2,1,3-benzoxadiazol-4-yl)-3H-spiro[2-benzofuran-1,4′-piperidin]-3-
one;
1′-[6-(diethylamino)-7-nitro-2,1,3-benzoxadiazol-4-yl]-3H-spiro[2-benzofuran-
1,4′-piperidin]-3-one;
1′-(7-nitro-6-piperidin-1-yl-2,1,3-benzoxadiazol-4-yl)-3H-spiro[2-benzofuran-
1,4′-piperidin]-3-one;
4-[3-(trifluoromethyl)phenyl]-2,1,3-benzoxadiazole;
4-(3-methylphenyl)-2,1,3-benzoxadiazole;
4-(4-tert-butylphenyl)-2,1,3-benzoxadiazole;
4-(3,5-difluorophenyl)-7-nitro-2,1,3-benzoxadiazole;
4-nitro-7-[3-(trifluoromethyl)phenyl]-2,1,3-benzoxadiazole;
4-(3-methylphenyl)-7-nitro-2,1,3-bonzoxadiazole;
4-nitro-7-[4-(trifluoromethyl)phenyl]-2,1,3-benzoxadiazole;
4-[2,4-bis(trifluoromethyl)phenyl]-7-nitro-2,1,3-benzoxadiazole;
4-(4-tert-butylphenyl)-7-nitro-2,1,3-benzoxadiazole;
4-(2-fluoro-3-methoxyphenyl)-7-nitro-2,1,3-benzoxadiazole;
4-{4-[3-chloro-5-(trifluoromethyl)pyridin-2-yl]piperazin-1-yl}-7-nitro-2,1,3-
benzoxadiazole;
methyl 4-({[6-(quinolin-2-ylthio)-2,1,3-benzoxadiazol-4-
yl]sulfonyl}amino)benzoate;
8-{[7-(4-phenyl-piperazin-1-sulfonamido)-2,1,3-benzoxadiazol-4-
yl]oxy}quinoline;
7-[4-(methylthio)phenyl]-4-nitro-2,1,3-benzoxadiazole;
7-(3-methoxyphenyl)-4-nitro-2,1,3-benzoxadiazole;
7-(3-fluorophenyl)-4-nitro-2,1,3-benzoxadiazole;
4-nitro-7-(quinolin-8-yl)-2,1,3-benzoxadiazole;
4-chloro-6-[3,5-(dimethyl)phenyl]-2,1,3-benzoxadiazole;
4,6-[3,5-(dimethoxy)phenyl]-2,1,3-benzoxadiazole;
4-chloro-6-[3,5-(difluoro)phenyl]-2,1,3-benzoxadiazole;
4-chloro-6-[3-(trifluoromethyl)phenyl]-2,1,3-benzoxadiazole;
4-chloro-6-(3-methylphenyl)-2,1,3-benzoxadiazole;
4-chloro-6-[4-(trifluoromethyl)phenyl]-2,1,3-benzoxadiazole;
4-chloro-6-(3-methoxyphenyl)-2,1,3-benzoxadiazole;
4-chloro-6-(4-(tert-butyl)phenyl)-2,1,3-benzoxadiazole;
4-chloro-6-(2-methoxyphenyl)-2,1,3-benzoxadiazole;
4-chloro-6-(3-nitrophenyl)-2,1,3-benzoxadiazole;
1′-{[7-chloro-(2,1,3-benzoxadiazol-4-yl)]sulfonyl}-3H-spiro[2-benzofuran-1,4′-
piperidin]-3-one;
1′-{(7-[naphth-1-ylthio]-2,1,3-benzoxadiazol-4-yl)}sulfonyl-3H-spiro[2-
benzofuran-1,4′-piperidine];
methyl 4-{[(7-{[4-hydroxyphenyl]thio}-2,1,3-benzoxadiazol-4-
yl)sulfonyl]amino}benzoate;
8-[(4-chloro-2,1,3-benzoxadiazol-6-yl)thio]quinoline;
1′-{[5-chloro-(2,1,3-benzoxadiazol-4-yl)]sulfonyl}-3H-spiro[2-benzofuran-1,4′-
piperidin]-3-one;
5-(1H-indazol-6-ylamino)-N-(1H-indazol-6-yl)2,1,3-benzoxadiazole-4-
sulfonamide;
5-hydroxy-N-(1H-indazol-6-yl)2,1,3-benzoxadiazole-4-sulfonamide;
5-[4-(2-{trifluoromethyl}phenoxy)piperidin-1-yl]-4-{[4-(2-
{trifluoromethyl}phenoxy)piperidin-1-yl]sulfonyl}-2,1,3-benzoxadiazole;
7-chloro-N-(4-chloro-2,5-dimethoxyphenyl)-2,1,3-benzoxadiazole-4-
sulfonamide;
N-(4-chloro-2,5-dimethoxyphenyl)-7-([4-chloro-2,5-dimethoxyphenyl]amino)-
2,1,3-benzoxadiazole-4-sulfonamide;
5-chloro-N-(4-chloro-2,5-dimethoxyphenyl)-2,1,3-benzoxadiazole-4-
sulfonamide;
N-(4-chloro-2,5-dimethoxyphenyl)-5-([4-chloro-2,5-dimethoxyphenyl]amino)-
2,1,3-benzoxadiazole-4-sulfonamide;
7-chloro-N-(2-fluorophenyl)-2,1,3-benzoxadiazole-4-sulfonamide;
N-(2-fluorophenyl)-7-(2-fluorophenylamino)-2,1,3-benzoxadiazole-4-
sulfonamide;
7-chloro-N-(2-isopropylphenyl)-2,1,3-benzoxadiazole-4-sulfonamide;
N-(4-chloro-2,5-dimethoxyphenyl)-7-fluoro-2,1,3-benzoxadiazole-4-
sulfonamide;
N-(4-chloro-2,5-dimethoxyphenyl)-7-([4-chloro-2,5-dimethoxyphenyl]amino)-
2,1,3-benzoxadiazole-4-sulfonamide;
7-chloro-N-(2-methyl-3-[trifluoromethyl]phenyl)-2,1,3-benzoxadiazole-4-
sulfonamide;
7-chloro-N-(5-fluoro-2-methylphenyl)-2,1,3-benzoxadiazole-4-sulfonamide;
7-chloro-N-(2-isopropyl-6-methylphenyl)-2,1,3-benzoxadiazole-4-sulfonamide;
7-chloro-N-(2,6-dimethylphenyl)-2,1,3-benzoxadiazole-4-sulfonamide;
7-chloro-N-(2-chloro-4-methylphenyl)-2,1,3-benzoxadiazole-4-sulfonamide;
7-chloro-N-mesityl-2,1,3-benzoxadiazole-4-sulfonamide;
7-chloro-N-(2-methoxyphenyl)-2,1,3-benzoxadiazole-4-sulfonamide;
7-chloro-N-(2,4,6-trifluorophenyl)-2,1,3-benzoxadiazole-4-sulfonamide;
7-chloro-N-(4-fluoro-2-methylphenyl)-2,1,3-benzoxadiazole-4-sulfonamide;
7-chloro-N-(2,5-dimethoxyphenyl)-2,1,3-benzoxadiazole-4-sulfonamide;
methyl 4-chloro-2-{[(7-chloro-2,1,3-benzoxadiazol-4-
yl)sulfonyl]amino}benzoate;
methyl 4-chloro-2-(7-[N-{5-chloro-2-(methoxycarbonyl)phenyl}sulfamoyl]-
2,1,3-benzoxadiazol-4-ylamino)benzoate;
7-chloro-N-(4-chloro-2-methoxy-5-methylphenyl)-2,1,3-benzoxadiazole-4-
sulfonamide;
7-chloro-N-(1H-indol-5-yl)-2,1,3-benzoxadiazole-4-sulfonamide;
7-chloro-N-(2,4-difluorophenyl)-2,1,3-benzoxadiazole-4-sulfonamide;
7-chloro-N-(2,5-dichlorophenyl)-2,1,3-benzoxadiazole-4-sulfonamide;
7-chloro-N-(2-[trifluoromethoxy]phenyl)-2,1,3-benzoxadiazole-4-sulfonamide;
N-(2,5-dimethoxyphenyl)-7-([2-methoxyphenyl]amino)-2,1,3-benzoxadiazole-4-
sulfonamide;
7-chloro-N-(5-methylisoxazol-3-yl)-2,1,3-benzoxadiazole-4-sulfonamide;
N-(2,5-dimethoxyphenyl)-7-(morpholin-4-yl)-2,1,3-benzoxadiazole-4-
sulfonamide;
7-chloro-N-(2-methyl-1H-indol-5-yl)-2,1,3-benzoxadiazole-4-sulfonamide;
N-(2,4-difluorophenyl)-7-([2,4-difluorophenyl]amino)-2,1,3-benzoxadiazole-4-
sulfonamide;
N-(2-fluorophenyl)-7-(morpholin-4-yl)-2,1,3-benzoxadiazole-4-sulfonamide;
7-nitro-N-(2-[trifluoromethoxy]phenyl)-2,1,3-benzoxadiazole-4-sulfonamide;
7-chloro-N-1,3-pyrimidin-4-yl-2,1,3-benzoxadiazole-4-sulfonamide;
7-(morpholin-4-yl)-2,1,3-benzoxadiazol-4-amine;
N-(2,3-difluorophenyl)-7-(quinolin-8-ylthio)-2,1,3-benzoxadiazole-4-
sulfonamide;
N-(2,3-difluorophenyl)-7-(pyrimidin-4-ylthio)-2,1,3-benzoxadiazole-4-
sulfonamide;
N-(2,3-difluorophenyl)-7-([2-methoxyphenyl]thio)-2,1,3-benzoxadiazole-4-
sulfonamide;
N-(2,3-difluorophenyl)-7-(naphthalen-1-ylthio)-2,1,3-benzoxadiazole-4-
sulfonamide;
N-(3-benzyloxypyridin-2-yl)-7-chloro-2,1,3-benzoxadiazole-4-sulfonamide;
7-chloro-N-(2-methoxy-5-methylphenyl)-2,1,3-benzoxadiazole-4-sulfonamide;
N-(3-benzyloxypyridin-2-yl)-7-(morpholin-4-yl)-2,1,3-benzoxadiazole-4-
sulfonamide;
7-chloro-N-(7-hydroxynaphthalen-1-yl)-2,1,3-benzoxadiazole-4-sulfonamide;
N-(2-methoxy-5-methylphenyl)-7-(quinolin-8-ylthio)-2,1,3-benzoxadiazole-4-
sulfonamide;
N-(7-hydroxynaphthalen-1-yl)-7-(morpholin-4-yl)-2,1,3-benzoxadiazole-4-
sulfonamide;
N-(2-methoxy-4-methylphenyl)-7-(morpholin-4-yl)-2,1,3-benzoxadiazole-4-
sulfonamide;
N-(5-aminonaphthalen-1-yl)-7-chloro-2,1,3-benzoxadiazole-4-sulfonamide;
7-chloro-N-(4-nitronaphthalen-1-yl)-2,1,3-benzoxadiazole-4-sulfonamide;
N-(5-aminonaphthalen-1-yl)-7-(morpholin-4-yl)-2,1,3-benzoxadiazole-4-
sulfonamide;
N-(5-aminonaphthalen-1-yl)-7-(quinolin-8-ylthio)-2,1,3-benzoxadiazole-4-
sulfonamide;
4-aminonaphthalen-1-yl 7-chloro-2,1,3-benzoxadiazole-4-sulfonate;
7-chloro-N-(naphthalen-1-yl)-2,1,3-benzoxadiazole-4-sulfonamide;
7-chloro-N-(4-hydroxynaphthalen-1-yl)-2,1,3-benzoxadiazole-4-sulfonamide;
4-(7-chloro-2,1,3-benzoxadiazole-4-sulfonamido)naphthalen-1-yl 7-chloro-2,1,3-
benzoxadiazole-4-sulfonate;
N-(4-fluorophenyl)-7-(morpholin-4-yl)-2,1,3-benzoxadiazole-4-sulfonamide;
7-{(2,3-difluorophenyl)amino}-2,1,3-benzoxadiazol-4-amine;
N-(2,3-difluorophenyl)-7-nitro-2,1,3-benzoxadiazol-4-amine;
N-(1H-indazol-5-yl)-2,1,3-benzoxadiazole-4,7-diamine;
4-(7-chloro-2,1,3-benzoxadiazole-4-sulfonamido)benzoic acid;
2-methoxy-N-(7-[{(2-trifluoromethoxy)phenyl}amino]-2,1,3-benzoxadiazol-4-
yl)benzamide;
N-(2-fluorophenyl)-7-nitro-2,1,3-benzoxadiazol-4-amine;
N-(2,3-difluorophenyl)-7-([4-hydroxyphenyl]thio)-2,1,3-benzoxadiazole-4-
sulfonamide;
N-(7-[{2,3-difluorophenyl}amino]-2,1,3-benzoxadiazol-4-yl)-2-
methoxybenzamide;
N-(2-fluorophenyl)-7-([2-methoxyphenyl]amino)-2,1,3-benzoxadiazole-4-
sulfonamide;
5,6-difluoro-4-(morpholin-4-yl)-2,1,3-benzoxadiazole;
4,5-difluoro-6-(morpholin-4-yl)-2,1,3-benzoxadiazole;
N-(naphthalen-1-yl)-7-(quinolin-8-ylthio)-2,1,3-benzoxadiazole-4-sulfonamide;
N-(2,3-dimethoxyphenyl)-5-([2,3-dimethoxyphenyl]amino)-2,1,3-
benzoxadiazole-4-sulfonamide;
5-chloro-N-(2,3-dimethoxyphenyl)-2,1,3-benzoxadiazole-4-sulfonamide;
7-chloro-N-(2,3-dimethoxyphenyl)-2,1,3-benzoxadiazole-4-sulfonamide;
5,7-dichloro-4-{(morpholin-4-yl)sulfonyl}-2,1,3-benzoxadiazole;
7-chloro-5-(morpholin-4-yl)-4-{(morpholin-4-yl)sulfonyl}-2,1,3-benzoxadiazole;
N-(2,3-dimethoxyphenyl)-7-(naphthalen-1-ylthio)-2,1,3-benzoxadiazole-4-
sulfonamide;
5,7-dicholoro-N-(2,6-difluorophenyl)-2,1,3-benzoxadiazole-4-sulfonamide;
7-chloro-N-(naphthalen-1-yl)-5-(napthalen-1-ylamino)-2,1,3-benzoxadiazole-4-
sulfonamide;
5,7-dichloro-N-(naphthalen-1-yl)-2,1,3-benzoxadiazole-4-sulfonamide;
5-chloro-N-(naphthalen-1-yl)-7-(naphthalen-1-ylamino)-2,1,3-benzoxadiazole-4-
sulfonamide;
4,6-bis(3-[trifluoromethyl]phenyl)-2,1,3-benzoxadiazole;
4,6-bis(3-methylphenyl)-2,1,3-benzoxadiazole;
4,6-bis(4-[trifluoromethyl]phenyl)-2,1,3-benzoxadiazole;
4,6-bis(3-methoxyphenyl)-2,1,3-benzoxadiazole;
4,6-bis(4-t-butylphenyl)-2,1,3-benzoxadiazole;
4,6-bis(2-methoxyphenyl)-2,1,3-benzoxadiazole;
N-(2-bromophenyl)-7-chloro-2,1,3-benzoxadiazole-4-sulfonamide;
4-(quinolin-8-ylthio)naphtho[2,1-c][1,2,5]oxadiazol-7-ol;
7-(3,5-dimethylphenyl)-2,1,3-benzoxadiazol-4-amine;
2,3-difluoro-N-(7-nitro-2,1,3-benzoxadiazol-4-yl)benzamide;
7-(3,5-difluorophenyl)-4-nitro-5-(piperidin-1-yl)-2,1,3,-benzoxadiazole;
7-(3-[trifluoromethyl]phenyl)-4-nitro-5-(piperidin-1-yl)-2,1,3-benzoxadiazole;
4-chloronaphtho[2,1-c][1,2,5]oxadiazol-7-ol;
7-(4-[trifluoromethyl]phenyl)-4-nitro-5-(piperidin-1-yl)-2,1,3-benzoxadiazole;
7-(3-methylphenyl)-4-nitro-5-(piperidin-1-yl)-2,1,3-benzoxadiazole;
7-(4-t-butylphenyl)-4-nitro-5-(piperidin-1-yl)-2,1,3-benzoxadiazole;
7-(3-methoxyphenyl)-4-nitro-5-(piperidin-1-yl)-2,1,3-benzoxadiazole;
7-(4-t-butylphenyl)-2,1,3-benzoxadiazol-4-amine;
N-(7-[3,5-dimethylphenyl]-2,1,3-benzoxadiazol-4-yl)acetamide;
7-(2-fluorophenyl)-4-nitro-2,1,3-benzoxadiazole;
7-chloro-N-(2-methoxyethyl)-2,1,3-benzoxadiazole-4-sulfonamide;
4-nitro-7-o-tolyl-2,1,3-benzoxadiazole;
4-nitro-7-phenyl-2,1,3-benzoxadiazole;
1-(4-[7-nitro-2,1,3-benzoxadiazol-4-yl]phenyl)ethanone;
4-nitro-7-(4-fluorophenyl)-2,1,3-benzoxadiazole;
1-(3-[7-nitro-2,1,3-benzoxadiazol-4-yl]phenyl)ethanone;
4-nitro-7-(3-nitrophenyl)-2,1,3-benzoxadiazole;
7-chloro-N-(3-phenylpropyl)-2,1,3-benzoxadiazole-4-sulfonamide;
7-chloro-N-(pyridin-2-ylmethyl)-2,1,3-benzoxadiazole-4-sulfonamide;
7-chloro-N-(cyclohexylmethyl)-2,1,3-benzoxadiazole-4-sulfonamide;
7-chloro-N-(cyclohexyl)-2,1,3-benzoxadiazole-4-sulfonamide;
7-chloro-N-(cyclopropylmethyl)-2,1,3-benzoxadiazole-4-sulfonamide;
4-(3,5-dimethylphenyl)-5-methoxy-2,1,3-benzoxadiazole;
4-cyclohexenyl-7-nitro-2,1,3-benzoxadiazole;
N-(2-fluorophenyl)-5-methyl-2,1,3-benzthiadiazole-4-sulfonamide;
N-(7-(4-t-butylphenyl)-2,1,3-benzoxadiazol-4-yl)acetamide;
4-(4-t-butylphenyl)naphtho[2,1-c][1,2,5]oxadiazol-7-ol;
7-chloro-N-(pyridin-4-ylmethyl)-2,1,3-benzoxadiazole-4-sulfonamide;
7-chloro-N-(3-[2-oxopyrrolidin-1-yl]propyl)-2,1,3-benzoxadiazole-4-
sulfonamide;
5-(2,3-dimethylphenyl)-6-fluoro-2,1,3-benzthiadiazole;
5-fluoro-6-phenyl-2,1,3-benzthiadiazole;
5-(3,4-dimethylphenyl)-6-fluoro-2,1,3-benzthiadiazole;
5-(benzodioxol-4-yl)-6-fluoro-2,1,3-benzthiadiazole;
5-(3,5-dimethylphenyl)-6-fluoro-2,1,3-benzthiadiazole;
5-(4-methoxyphenyl)-6-fluoro-2,1,3-benzthiadiazole;
5-(3,4-dimethoxyphenyl)-6-fluoro-2,1,3-benzthiadiazole;
4-nitro-7-(4-phenoxyphenyl)-2,1,3-benzoxadiazole;
4-(3,4-dimethoxyphenyl)-7-nitro-2,1,3-benzoxadiazole;
4-(4-methoxyphenyl)-7-nitro-2,1,3-benzoxadiazole;
4-(benzodioxol-5-yl)-7-nitro-2,1,3-benzoxadiazole;
4-(4-methylphenyl)-7-nitro-2,1,3-benzoxadiazole;
4-(naphthalen-1-yl)-7-nitro-2,1,3-benzoxadiazole;
4-(2,3-dimethylphenyl)-7-nitro-2,1,3-benzoxadiazole;
4-(2,5-dimethylphenyl)-7-nitro-2,1,3-benzoxadiazole;
4-(3,4-dimethylphenyl)-7-nitro-2,1,3-benzoxadiazole;
4-(2,3-dimethylphenyl)-6-(trifluoromethyl)-2,1,3-benzoxadiazole;
4-phenyl-6-(trifluoromethyl)-2,1,3-benzoxadiazole;
4-(benzodioxol-5-yl)-6-(trifluoromethyl)-2,1,3-benzoxadiazole;
6-nitro-4-p-tolyl-2,1,3-benzoxadiazole;
4-(2,4-bis[trifluoromethyl]phenyl)-6-nitro-2,1,3-benzoxadiazole;
6-nitro-4-m-tolyl-2,1,3-benzoxadiazole;
4-(3,5-difluorophenyl)-6-nitro-2,1,3-benzoxadiazole;
4-(3-[trifluoromethyl]phenyl)-6-nitro-2,1,3-benzoxadiazole;
4-(4-t-butylphenyl)-6-nitro-2,1,3-benzoxadiazole;
4-(2-methoxyphenyl)-6-nitro-2,1,3-benzoxadiazole;
6-nitro-4-(quinolin-8-yl)-2,1,3-benzoxadiazole;
4-(2,5-dimethylphenyl)-6-nitro-2,1,3-benzoxadiazole;
4-(4-chlorophenyl)-1-({7-(quinolin-2ylthio)-2,1,3-benzoxadiazol-4-
yl}sulfonyl)piperidin-4-ol;
N-(2,1,3-benzoxadiazol-4-yl)-1-(3-chloropyridin-2-yl) azetidine-3-carboxamide;
and
pharmaceutically acceptable salts, hydrates, solvates, polymorphs, atrophisomers,
N-oxides, and prodrugs thereof.

14. A composition comprising, together with a pharmaceutically acceptable carrier, diluent, or excipient, a compound (or one of its pharmaceutically acceptable salts, hydrates, solvates, polymorphs, atrophisomers, N-oxides, and prodrugs) according to any one of claims 1 parts (A) and (C) (except subpart (f)), 2, 6, 12, and 13.

15. A method of inhibiting ubiquitination in a cell comprising contacting the cell with a compound according to any one of claims 1 parts (A) and (C) (except subpart (f)), 2, 6, 12, and 13.

16. A method of inhibiting ubiquitination in a cell comprising contacting the cell with a composition according to any one of claims 1 parts (A) and (C) (except subpart (f)), 2, 6, 12, and 13.

17. The method according to claim 15, wherein the cell is an animal cell.

18. The method according to claim 16, wherein the cell is an animal cell.

19. The method according to claim 17, wherein the animal cell is derived from a mammal.

20. The method according to claim 18, wherein the animal cell is derived from a mammal.

21. A method of treating cell proliferative diseases or conditions comprising administering to a patient an effective amount of a composition comprising, together with a pharmaceutically acceptable carrier, diluent, or excipient, a compound (or one of its pharmaceutically acceptable salts, hydrates, solvates, polymorphs, atrophisomers, N-oxides, and prodrugs) according to any one of claims 1 parts (A) and (C), 2, 6, 12, and 13.

22. The method according to claim 21, wherein the cell proliferative diseases are cancers.

23. A method of inhibiting TRAF6, comprising administering to a patient in need thereof an effective amount of a composition comprising, together with a pharmaceutically acceptable carrier, diluent, or excipient, a compound (or one of its pharmaceutically acceptable salts, hydrates, solvates, polymorphs, atrophisomers, N-oxides, and prodrugs) according to any one of claims 1 parts (A) and (C), 2, 6, 12, and 13.

24. The method according to claim 23, wherein the patient suffers from a condition or disease that involves a process selected from the group consisting of cancer, inflammation, adaptive immunity, innate immunity, bone metabolism, LPS-induced angiogenesis, osteoporosis, osteopinneal diseases, lymph node development, mammary gland development, skin development, and central nervous system development.
